(19) United States
(12) Patent Application Publication
(10) Pub. No.: US 2012/0165796 A1
(43) Pub. Date: Jun. 28, 2012

(54) Pill Catchers

(75) Inventors: Mark S. Ortiz, Milford, OH (US); Christopher J. Hess, Cincinnati, OH (US); Jason L. Harris, Mason, OH (US); James W. Voegele, Cincinnati, OH (US); William B. Weisenburgh, II, Maineville, OH (US); Mark S. Zeiner, Mason, OH (US); Mark D. Overmyer, Cincinnati, OH (US)

(73) Assignee: ETHICON ENDO-SURGERY, INC., Cincinnati, OH (US)

(21) Appl. No.: 12/976,726

(22) Filed: Dec. 22, 2010

(51) Int. Cl. A61M 31/00

(52) U.S. Cl. 604/891.1; 604/890.1

(57) ABSTRACT

Devices and related methods are provided for the controlled delivery of a therapeutic to a targeted location within a body. More particularly, methods and devices are provided for controlling the rate of passage of an orally administered pill through a body, as well as for controlling the delivery of a therapeutic within the pill at a specific location within the body. Various types of devices, generally referred to herein as “catchers,” are provided that can actively catch a pill as it passes through a body. The catcher can hold the pill at a specific location within the body until a predetermined event occurs, such as partial or complete administration of a therapeutic within the pill. The catcher can then release the pill upon command and/or upon the occurrence of the predetermined event to allow the pill to pass out of the body. In other embodiments, various types of pills are provided that can actively engage a catcher and remain engaged with the catcher until a predetermined event occurs.

### FIELD

The present invention relates to methods and devices for delivering drugs, and in particular to methods and devices for controlling drug delivery to a targeted region of the body.

### BACKGROUND

Metabolic disorders such as obesity have many facets and causes. Attempts to address these maladies with narrow spectrum solutions often do not succeed because of the body's complexity. Treatments that address these disorders by triggering receptors in one part of the body, often also trigger receptors in another part of the body causing undesirable broad spectrum side affects. From time to time, it is desirable to deliver a targeted dose of a therapeutic to a particular region of the body. Current techniques, however, fall short of being able to deliver regular and predictable targeted doses noninvasively.

For example, implants may be capable of delivering a targeted dose, but require refills. If the implant is located in the gastrointestinal tract, refilling typically requires invasive techniques that are undesirable on a regular basis. A percutaneous fill port can also be used for targeted dosing, but chronic and/or regular punctures of the lumen wall can lead to hazardous infections, biofilms, or injury to the lumen wall. Time release pills fall short of truly targeting a zone of the body because transit rates differ vastly from person to person, as well as in a given person depending on diet (e.g., high versus low fiber). Verification that the proper therapeutic has been released to the correct location is impossible with time release pills. Further, patient compliance is low for regular frequent administration of pills.

Accordingly, there is a need for methods and devices directed to delivering a targeted, verifiable, and scheduled dose of a therapeutic or suite of therapeutics without the problems associated with the above mentioned modalities.

### SUMMARY

The devices and methods disclosed herein generally involve controlling the delivery of a therapeutic to a targeted location within a body. More particularly, methods and devices are provided for controlling the timing of delivery of a therapeutic within an orally administered pill, catching the pill at a specific location within a body, holding the pill until the administration of the therapeutic has been completed, and releasing the pill upon command and/or upon the occurrence of a predetermined event.

In one aspect, a device for controlled therapeutic drug delivery within a patient is provided and can include an anchor configured to be disposed within a patient's digestive tract and having a catch mechanism that is movable between a first configuration in which the catch mechanism is effective to capture a pill swallowed by a patient to prevent passage of the pill through the catch mechanism, and a second configuration in which the catch mechanism releases the pill to allow passage of the pill through the catch mechanism. In some embodiments, the anchor can include a body having a tether extending distally therefrom, and the catch mechanism can be coupled to a distal end of the tether.

In one embodiment, the catch mechanism can be configured to move between the first configuration and the second configuration in response to a triggering signal. For example, the catch mechanism can be coiled in the first configuration to capture a pill and straightened in the second configuration to release the pill. The catch mechanism can be formed from any suitable material known in the art, including but not limited to, a shape memory material such as Nitinol. In some embodiments, the anchor can include a sleeve having a lumen extending therethrough, and the catch mechanism can be disposed within the lumen. The anchor can also include a cylindrical sleeve configured to be fixed within a patient's digestive tract and configured to funnel a pill into the catch mechanism. The catch mechanism can optionally be positioned on a distal end of the cylindrical sleeve and can be configured to contract in the first configuration to retain a pill and expand in the second configuration to release the pill. In some embodiments, the catch mechanism can include an expandable iris that is contracted in the first configuration to retain a pill and that is dilated in the second configuration to allow passage of the pill.

In another aspect, a system for controlled therapeutic drug delivery within a patient is provided and can include a pill configured to deliver at least one therapeutic drug and configured to be swallowed by a patient. The system can also include a catch mechanism configured to be disposed within a patient's digestive tract. In some embodiments, the catch mechanism can be movable between a first configuration in which the catch mechanism is effective to capture the pill and prevent passage of the pill through the catch mechanism, and a second configuration in which the catch mechanism is effective to release the pill to allow passage of the pill through the catch mechanism.

The system can also include an anchor configured to retain the catch mechanism within a patient's gastrointestinal tract. For example, the anchor can be a substantially rigid ring configured to be disposed within a patient's stomach and having a size large enough to prevent passage thereof through a patient's pylorus. In some embodiments the system can further include a tether extending from the anchor and having the catch mechanism disposed on a distal end thereof. In other embodiments, the system can include an actuator mechanism configured to move the catch mechanism from the first configuration to the second configuration. The actuator mechanism can take many forms, for example, a transcutaneous energy transfer system and/or a motor.

In a further aspect, a method for controlled therapeutic drug delivery is provided and can include introducing a pill orally into a digestive tract such that the pill is captured by a catch mechanism positioned in a first configuration disposed within the digestive tract. The pill can deliver a therapeutic drug directly into the patient's digestive tract. In some embodiments, after a predetermined event, the catch mechanism can move to a second configuration in which the pill is released and allowed to pass through the digestive tract. For example, the catch mechanism can move between a first configuration in which it includes features to catch the pill and prevent it from passing further through the digestive tract and a second configuration in which features allow the pill to pass through the catch mechanism and to be released. The predetermined event can take many forms including a predetermined amount of time and/or a triggering signal such as a signal delivered to the catch mechanism from an external source. The triggering signal can be generated when all of the therapeutic drug disposed in the pill has been delivered.

In still further aspects, a device for controlled therapeutic drug delivery within a patient is provided and can include a pill configured to pass through a patient's digestive tract and to deliver a therapeutic agent directly into the patient's digestive tract. The pill can have a first configuration in which the pill is configured to engage a catch disposed within a patient's digestive tract, and a second configuration in which the pill is configured to release from the catch. In some embodiments, the pill can have retractable arms that are configured to be expanded in the first configuration to engage the catch and retracted in the second configuration to disengage from the catch. The pill can also include a coating disposed therearound that maintains the pill in the second configuration and can dissolve to allow the pill to move to the first configuration.

In one embodiment, the pill can include a catch engagement mechanism that extends outward from the pill when the pill is in the first configuration, and that is retracted into the pill as the therapeutic agent is delivered into the patient's digestive tract such that the pill moves into the second configuration and is released from the catch. The pill can optionally include a plurality of arms that extend outward from the pill for engaging the catch when the pill is in the first configuration. In one embodiment, the pill can include a plurality of absorbable arms that extend from the pill for engaging the catch when the pill is in the first configuration, and that are configured to be absorbed by a patient's body to move the pill to the second configuration. The pill can also include one or more openings formed therein and configured to engage the catch in the first configuration. In some embodiments, axial rotation of the pill is effective to move the pill to the second configuration.

In one aspect, a system for controlled therapeutic drug delivery within a patient is provided and can include a catch mechanism configured to be anchored in a patient's digestive tract, and a pill configured to pass through a patient's digestive tract. The pill can have a first configuration in which the pill is retained by the catch mechanism, and a second configuration in which the pill releases from the catch mechanism. In some embodiments, the catch mechanism can include a basket having a plurality of engagement members configured to engage and retain the pill in the first configuration. Further, the pill can optionally have a plurality of arms that can be retained within the pill by a coating when the pill is in the second configuration. The coating can be configured to dissolve to release the arms and move the pill to the first configuration.

The system can also include an anchor configured to retain the catch mechanism within a patient's gastrointestinal tract. In addition, the system can include a tether extending between the anchor and the catch mechanism. In some embodiments, the anchor can include a substantially rigid ring configured to be disposed within a patient's stomach and having a size large enough to prevent passage of the ring through a patient's pylorus. In the alternative, the anchor can include a stent having a plurality of tissue engaging tines configured to engage tissue within a patient's digestive system to anchor the stent. In one embodiment, the system can also include an actuator mechanism configured to move the pill from the first configuration to the second configuration.

In a further aspect, a method for controlled therapeutic drug delivery is provided and can include introducing a pill orally into a digestive tract such that the pill engages a catch disposed within the digestive tract and is retained by the catch. The pill can then deliver a therapeutic drug directly into the patient's digestive tract and can be released from the catch after a predetermined event. In some embodiments, the predetermined event can include delivery of an external triggering signal to the pill. The predetermined event can also include disintegration of a catch engagement mechanism on the pill that allows the pill to release from the catch. In the alternative, the predetermined event can include a triggering signal when all of the therapeutic drug disposed in the pill has been delivered. In one embodiment, the method can also include, prior to the step of introducing the pill, implanting an anchor within a patient's digestive tract, the anchor having the catch coupled thereto.

In an additional aspect, a device for controlled therapeutic drug delivery within a patient is provided and can include an anchor configured to be disposed within a patient's digestive tract and having a catch mechanism coupled thereto. In some embodiments, the catch mechanism can have a first configuration in which a magnet on the catch mechanism is configured to magnetically engage a pill swallowed by a patient to prevent passage of the pill through the catch mechanism, and a second configuration in which the magnet is configured to release the pill to allow passage of the pill through the catch mechanism. The device can also include a tether extending between the anchor and the catch mechanism.

In one embodiment, the catch mechanism can include a housing having a proximal end and a distal end with a longitudinal axis extending therebetween. The magnet can be disposed within the housing and can be movable along the longitudinal axis of the housing. In the first configuration the magnet can be disposed distally within the housing to attract a pill and prevent passage of the pill. In the second configuration, the magnet can be disposed proximally within the housing to release the pill and allow it to pass. In some embodiments, the catch mechanism can include a shape memory alloy disposed therein and configured to move the magnet between the first configuration and the second configuration in response to a change in energy application thereto. In other embodiments, the catch mechanism can include a step motor disposed therein configured to move the magnet between the first configuration and the second configuration in response to a change in energy application thereto.

The catch mechanism can include a transcutaneous energy transfer coil configured to receive an external signal to supply energy to a mechanism for moving the magnet between the first configuration and the second configuration. The housing can optionally include a distal non-ferromagnetic portion configured to allow a magnetic field of the magnet to extend outside of the housing when the catch mechanism is in the first configuration to attract a pill, and a proximal ferromagnetic portion configured to prevent the magnetic field from extending outside of the housing when the catch mechanism is in the second configuration to prevent attraction of a pill.

In other aspects, a system for controlled therapeutic drug delivery within a patient is provided and can include a pill configured to deliver at least one therapeutic drug and configured to be swallowed by a patient. The system can also include a catch mechanism configured to be disposed within a patient's digestive tract. The catch mechanism can be movable between a first configuration in which the catch mechanism is configured to magnetically engage the pill and prevent passage of the pill through the catch mechanism, and a second configuration in which the catch mechanism is configured to release the pill to allow passage of the pill through the catch mechanism. In some embodiments, the pill can include a ferromagnetic material configured to be magnetically engaged with the catch mechanism in the first configuration.

In some embodiments, the system can include an anchor configured to be disposed within a patient's digestive tract and configured to retain the catch mechanism within the patient's digestive tract. The anchor can have many configurations, for example the anchor can be a substantially rigid ring configured to be disposed within a patient's stomach and can have a size large enough to prevent passage of the ring through a patient's pylorus. In some embodiments, the catch mechanism can include a magnet disposed within a housing. In the first configuration the magnet can be disposed distally within the housing to magnetically engage the pill and prevent passage of the pill, and in the second configuration the magnet can be disposed proximally within the housing to release the pill and allow it to pass. The catch mechanism can also include a distal non-ferromagnetic portion configured to allow a magnetic field of a magnet to extend outside of the catch mechanism when in the first configuration to attract the pill, and a proximal ferromagnetic portion configured to prevent the magnetic field from extending outside of the catch mechanism when in the second configuration to prevent attraction of the pill.

In a further aspect, a method for controlled therapeutic drug delivery is provided and can include implanting an anchor having a catch mechanism in a patient's digestive tract, the catch mechanism being positioned in a first configuration in which the catch mechanism is effective to magnetically engage a pill and prevent passage of the pill through the catch mechanism. The catch mechanism can be movable to a second configuration in which the catch mechanism releases the pill to allow passage of the pill through the catch mechanism. The catch mechanism can optionally include a magnet that moves within a housing between a first configuration and a second configuration. A magnetic field of the magnet can extend outside of the catch mechanism in the first configuration and can be prevented from extending outside of the catch mechanism in the second configuration. The method can also include actuating an external source to supply energy to the catch mechanism to move the catch mechanism between the first configuration and the second configuration. In some embodiments, a pill can be introduced orally into a digestive tract and can engage the catch mechanism. The pill can deliver a therapeutic drug directly into the patient's digestive tract when engaged with the catch mechanism.

In other aspects, a system for controlled therapeutic drug delivery within a patient is provided and can include a catch mechanism configured to be anchored in a patient's digestive tract, and a pill configured to pass through a patient's digestive tract. The pill can have a first configuration in which the pill is magnetically engaged by the catch mechanism, and a second configuration in which the pill is released from the catch mechanism. In some embodiments, the system can also include an anchor configured to be disposed within a patient's digestive tract to retain the catch mechanism within the patient's digestive tract, and a tether extending between the anchor and the catch mechanism. The anchor can optionally be a substantially rigid ring configured to be disposed within a patient's stomach and having a size large enough to prevent passage of the ring through a patient's pylorus.

The catch mechanism can have many configurations, and in one embodiment, the catch mechanism can include a ferromagnetic material configured to magnetically engage the pill in the first configuration. The catch mechanism can also include an alignment mechanism and a sheath. The alignment mechanism can be configured to funnel the pill into the sheath. The catch mechanism can also include a sheath and a ferromagnetic receiving platform. The sheath can optionally be configured to funnel the pill onto the receiving platform. In one embodiment, the pill can include a non-ferromagnetic portion configured to allow a magnetic field of the pill to extend outside of the pill to engage the catch mechanism in the first configuration, and a ferromagnetic portion configured to prevent the magnetic field from extending outside of the pill in the second configuration so that the pill is released from the catch mechanism.

In one aspect, a device for controlled therapeutic drug delivery within a patient is provided and can include a pill configured to pass through a patient's digestive tract and to deliver a therapeutic agent directly into the patient's digestive tract. The pill can have a magnet associated therewith and configured to magnetically engage a catch mechanism disposed within a patient's digestive tract to retain the pill in a substantially fixed position within the digestive tract. In some embodiments, the magnet can have a first position in which it is magnetically attracted to a catch mechanism and a second position in which it is not magnetically attracted to the catch mechanism. In other embodiments, the magnet can be disposed within the pill and can be rotatable about a central longitudinal axis of the pill to move the pill between the first configuration and the second configuration.

The pill can have many different configurations, and in one embodiment, the pill can include an absorbable material that maintains the magnet in a first configuration such that the magnet is attracted to the catch mechanism. The absorbable material can be configured to dissolve to allow the magnet to move to a second configuration in which a magnetic field of the magnet is blocked. The magnet can also be disposed within a non-ferromagnetic portion of the pill in the first position and can be disposed within a ferromagnetic portion of the pill in the second position. The pill can optionally include a piston disposed therein configured to move along a central longitudinal axis of the pill to dispense a therapeutic agent into a patient's digestive tract. The piston can be configured to move the magnet along the central longitudinal axis of the pill.

In a further aspect, a method for controlled therapeutic drug delivery is provided and can include introducing a pill orally into a digestive tract. The pill can have a magnet that magnetically engages a catch mechanism disposed within the digestive tract such that the pill is retained by the catch mechanism. In addition, the pill can move to a second configuration after a therapeutic drug is delivered directly into the patient's digestive tract such that the pill releases from the catch mechanism. In some embodiments, the method can also include, prior to introducing the pill, implanting an anchor in a patient's digestive tract. The anchor can have a tether extending therefrom to retain the catch mechanism. Furthermore, the magnet can move relative to the pill to move the pill between the first configuration and the second configuration.

In one embodiment, the magnet can remain stationary relative to the pill as the pill moves between the first configuration and the second configuration. The pill can also slide along a tether into a ferromagnetic sheath of the catch mechanism. In some embodiments, a piston assembly within the pill can dispense the therapeutic agent and can move the pill to the second configuration.

### BRIEF DESCRIPTION OF THE DRAWINGS

The invention will be more fully understood from the following detailed description taken in conjunction with the accompanying drawings, in which:

FIG. 1 is a schematic view of one exemplary embodiment of a gastric ring anchor positioned in a digestive tract;

FIG. 2A is a perspective view of one exemplary embodiment of a peristalsis-resisting platform having proximal and distal fingers;

FIG. 2B is a cross-sectional view of the platform of FIG. 2A installed in a body lumen;

FIG. 2C is another cross-sectional view of the platform of FIG. 2A installed in a body lumen;

FIG. 3A is a perspective view of one exemplary embodiment of a peristalsis-resisting platform having proximal and distal bladders;

FIG. 3B is a cross-sectional view of the platform of FIG. 3A installed in a body lumen;

FIG. 3C is a another cross-sectional view of the platform of FIG. 3A installed in a body lumen;

FIG. 4 is a schematic view of one exemplary embodiment of a stent anchor positioned in a digestive tract;

FIG. 5 is a schematic view of one exemplary embodiment of a sleeve anchor positioned in a digestive tract;

FIG. 6A is a cross-sectional view of a digestive tract having a tethered catcher disposed therein;

FIG. 6B is another cross-sectional view of a digestive tract having a tethered catcher disposed therein;

FIG. 7A is a perspective view of one exemplary embodiment of a pig-tail catcher;

FIG. 7B is a perspective view of a pill and the pig-tail catcher of FIG. 7A shown in a first configuration;

FIG. 7C is a perspective view of the pig-tail catcher of FIGS. 7A-7B shown in a second configuration;

FIG. 8A is a side view of another exemplary embodiment of a pig-tail catcher shown in a first configuration;

FIG. 8B is a side view of the pig-tail catcher of FIG. 8A shown in a second configuration;

FIG. 9A is a perspective view of a pill and one exemplary embodiment of a sleeve catcher;

FIG. 9B is a perspective view of a clamp element of the sleeve catcher of FIG. 9A shown in a first configuration;

FIG. 9C is a perspective view of the clamp element of FIG. 9B shown in a second configuration;

FIG. 10A is a perspective view of one exemplary embodiment of an iris catcher shown in a first configuration;

FIG. 10B is a perspective view of the iris catcher of FIG. 10A shown in a second configuration

FIG. 11A is a perspective view of one exemplary embodiment of a pill having an expandable frame shown in a first configuration;

FIG. 11B is a perspective view of the pill of FIG. 11A shown in a second configuration;

FIG. 12A is a schematic view of one exemplary embodiment of a pill having deployable wings;

FIG. 12B is a perspective view of one exemplary embodiment of a catcher configured to catch the pill of FIG. 12A;

FIG. 12C is a perspective view of the pill of FIG. 12A and the catcher of FIG. 12B;

FIG. 13A is a perspective view of one exemplary embodiment of a pill having deployable arms shown in a second configuration;

FIG. 13B is an end view of the pill of FIG. 13A shown in the second configuration;

FIG. 13C is a perspective view of the pill of FIGS. 13A-13B shown in a first configuration;

FIG. 13D is an end view of the pill of FIGS. 13A-13C shown in the first configuration;

FIG. 14A is a schematic view of one exemplary embodiment of a pill having a deployable adhesive;

FIG. 14B is a schematic view of the pill of FIG. 14A with the adhesive deployed;

FIG. 14C is a time-series schematic view of the pill of FIGS. 14A-14B positioned at various points within a digestive tract;

FIG. 15A is a schematic view of one exemplary embodiment of a pill having deployable legs;

FIG. 15B is a time-series schematic view of the pill of FIG. 15A positioned at various points within a digestive tract;

FIG. 16A is a plan view of one exemplary embodiment of a pill having a catch-engaging member and a dissolvable coating;

FIG. 16B is a plan view of the pill of FIG. 16A shown with the coating dissolved;

FIG. 17A is a cross-sectional schematic view of one exemplary embodiment of a pill having deployable and retractable hooks;

FIG. 17B is a cross-sectional schematic view of the pill of FIG. 17A shown with the hooks fully deployed;

FIG. 17C is an end view of the pill of FIGS. 17A-17B shown with the hooks fully deployed;

FIG. 17D is a cross-sectional schematic view of the pill of FIGS. 17A-17C shown with the hooks partially retracted;

FIG. 17E is a cross-sectional schematic view of the pill of FIGS. 17A-17D shown with the hooks fully retracted;

FIG. 17F is a cross-sectional schematic view of the pill of FIGS. 17A-17E shown with the hooks collapsed before being fully retracted;

FIG. 18A is a perspective view of one exemplary embodiment of a pill configured to lodge in a digestive tract;

FIG. 18B is a perspective view of one exemplary embodiment of a pill having an expandable foam portion;

FIG. 18C is a perspective view of the pill of FIG. 18B shown expanded within a body lumen;

FIG. 19A is a perspective view of one exemplary embodiment of a pill having bioabsorbable suture loops;

FIG. 19B is a perspective view of one exemplary embodiment of a catcher configured to catch the pill of FIG. 19A;

FIG. 19C is a partial cross-sectional perspective view of the pill of FIG. 19A and the catcher of FIG. 19B disposed within a body lumen;

FIG. 20A is a plan view of one exemplary embodiment of a pill having dissolvable suture loops;

FIG. 20B is a plan view of the pill of FIG. 20A and one exemplary embodiment of a catcher configured to catch the pill of FIG. 20A disposed within a body lumen;

FIG. 20C is a perspective view of one exemplary embodiment of a pill catching system that includes pills and tethers having corresponding hook and loop elements;

FIG. 21A is a plan view of one exemplary embodiment of a pill having a dissolvable grappling hook;

FIG. 21B is a plan view of one exemplary embodiment of a pill having a dissolvable loop;

FIG. 21C is a schematic view of one exemplary embodiment of a pill having a tether release mechanism;

FIG. 22A is a plan view of one exemplary embodiment of a pill having dissolvable hooks and a dissolvable coating;

FIG. 22B is a perspective view of a catcher configured to catch the pill of FIG. 22A;

FIG. 22C is a plan view of one exemplary embodiment of a pill having deployable hooks shown with the hooks retracted;

FIG. 22D is a schematic view of the pill of FIG. 22C shown with the hooks deployed;

FIG. 23A is a perspective view of one exemplary embodiment of a spring and ball pill catching system;

FIG. 23B is a schematic view of one exemplary embodiment of a coil spring pill catching system;

FIG. 23C is another schematic view of the coil spring pill catching system of FIG. 23B;

FIG. 23D is a schematic view of one exemplary embodiment of a pill having a deployable stent shown positioned within a digestive tract;

FIG. 24A is a perspective view of one exemplary embodiment of a pill having a rotation lock shown in a first configuration, a second half of the pill being shown in phantom;

FIG. 24B is a perspective view of the pill of FIG. 24A shown in a second configuration, the second half of the pill being shown in phantom;

FIG. 24C is a plan view of the pill of FIGS. 24A-24B and one exemplary embodiment of a catcher configured to catch the pill of FIGS. 24A-24B;

FIG. 24D is an end view of the catcher of FIG. 24C and the pill of FIGS. 24A-24C;

FIG. 24E is a perspective view of the catcher of FIGS. 24C-24D and the pill of FIGS. 24A-24D;

FIG. 24F is a top view of one exemplary embodiment of a locking pin that can be used with the pill of FIGS. 24A-24E;

FIG. 24G is a side view of the locking pin of FIG. 24F;

FIG. 25A is a cross-sectional view of one exemplary embodiment of a pill having an absorbable rib and one exemplary embodiment of a corresponding catcher;

FIG. 25B is an end view of the catcher of FIG. 25A;

FIG. 26A is a time-series perspective view of a pill passing through one exemplary embodiment of a sleeve catcher having a ring stopper;

FIG. 26B is a time-series perspective view of a pill passing through the sleeve catcher of FIG. 26A shown positioned within a digestive tract;

FIG. 27A is a partial cross-sectional perspective view of one exemplary embodiment of a slotted tube catcher and a pill shown in a first configuration;

FIG. 27B is a partial cross-sectional side view of the catcher and pill of FIG. 27A;

FIG. 27C is an end view of the catcher and pill of FIGS. 27A-27B with the pill shown in the first configuration;

FIG. 27D is an end view of the catcher and pill of FIGS. 27A-27C with the pill shown in a second configuration;

FIG. 28A is a perspective view of an exemplary magnetic base;

FIG. 28B is a schematic of the magnetic base of FIG. 28A in the off configuration;

FIG. 28C is a schematic of the magnetic base of FIG. 28A in the on configuration;

FIG. 29A is a side view of one exemplary embodiment of a magnetic pill catcher system having an active magnetic catcher and a pill;

FIG. 29B is a perspective view of the catcher of FIG. 29A;

FIG. 29C is a perspective view of the pill of FIG. 29A;

FIG. 29D is a cross-sectional view of the catcher and the pill of FIG. 29A;

FIG. 30A is a cross-sectional view of an exemplary pill catcher system in a first configuration;

FIG. 30B is a cross-sectional view of the pill catcher system of FIG. 30A in a second configuration;

FIG. 30C is a side view of the pill catcher system of FIG. 30A showing an exemplary TET coil coupled to an anchor within a patient's stomach;

FIG. 31A is a cross-sectional view of another pill catcher system with a shape memory alloy actuator for axially moving a magnet disposed therein;

FIG. 31B is a cross-sectional view of an exemplary TET coil;

FIG. 32A is a perspective view of an exemplary embodiment of a magnet;

FIG. 32B is a perspective view of another exemplary embodiment of a magnet;

FIG. 32C is a perspective view of another exemplary embodiment of a magnet;

FIG. 33 is a perspective view of one embodiment of a pill catcher system with a subcutaneous access port;

FIG. 34 is a perspective view of another exemplary embodiment of a pill catcher system having a weighted sleeve for catching a pill;

FIG. 35 is a perspective view of one exemplary embodiment of a pill catcher system having a ferromagnetic ring and a pill with a catheter dispenser;

FIG. 36A is a side view of one embodiment of a pill catcher system having a sleeve for funneling a pill into a magnetic catcher platform;

FIG. 36B is a perspective view of the pill catcher system of FIG. 36A

FIG. 36C is a perspective view of the catcher platform of FIG. 36A;

FIG. 36D is a perspective view of the pill of FIG. 36A docked to the catcher platform;

FIG. 36E is a perspective view of another embodiment of a pill catcher system having a movable magnet in a first configuration;

FIG. 36F is a side view of the system of FIG. 36A in a second configuration;

FIG. 37A is a perspective view of another exemplary embodiment of a pill catcher system having a magnetic helical tether for funneling a pill into a catcher;

FIG. 37B is a perspective view of the helical tether and the catcher of FIG. 37A;

FIG. 37C is a perspective view of the pill of FIG. 37A;

FIG. 37D is a perspective view of the pill traveling along the helical tether of FIG. 37A;

FIG. 37E is a perspective view of the pill within the catcher of FIG. 37A

FIG. 37F is a perspective view of a second pill traveling along the helical tether to push the pill out of the catcher of FIG. 37A;

FIG. 37G is a perspective view of the second pill of FIG. 37F pushing the pill out of the catcher;

FIG. 38A is a side view of one embodiment of a pill catcher system having a catcher with a concave distal end;

FIG. 38B is a cross-sectional view of the catcher of FIG. 38A and a magnetic pill;

FIG. 38C is a cross-sectional view of the catcher of FIG. 38A and the pill of FIG. 38B nearing a magnetic distal end of the catcher

FIG. 38D is a cross-sectional view of the catcher of FIG. 38A and the pill of FIG. 38B being moved by peristalsis;

FIG. 38E is a cross-sectional view of the catcher of FIG. 38A and the pill of FIG. 38B as the pill is about to dock with the catcher;

FIG. 38F is a cross-sectional view of the pill of FIG. 38B docked with the catcher of FIG. 38A;

FIG. 38G is a cross-sectional view of the catcher of FIG. 38A as the pill of FIG. 38B releases from the catcher;

FIG. 38H is a cross-sectional side view of the pill of FIG. 38B illustrating an exemplary magnetic switching mechanism;

FIG. 38I is a perspective view of the pill of FIG. 38H in a first configuration;

FIG. 38J is a perspective view of the pill of FIG. 38H as it moves to a second configuration;

FIG. 38K is a perspective view of the pill of FIG. 38H in the second configuration;

FIG. 39 is a side view of another embodiment of a pill catcher system having a magnetic ring and a switchable magnetic hook;

FIG. 40A is a cross-sectional view of one embodiment of a pill having a magnetic switching mechanism in a first configuration;

FIG. 40B is a cross-sectional view of the pill of FIG. 40A in a second configuration;

FIG. 41A is a perspective view of a ferromagnetic ring disposed around a lumen of a patient's G.I. tract;

FIG. 41B is a cross-sectional view of the ring of a FIG. 41A and the pill of FIG. 40A traveling through a patient's G.I. tract;

FIG. 41C is a cross-sectional view of the ring of a FIG. 41A and the pill of FIG. 40A docking with the lumen;

FIG. 41D is a cross-sectional view of the ring of FIG. 41A and the pill of FIG. 40A moving to the second configuration;

FIG. 41E is a cross-sectional view of the ring of FIG. 41A and the pill of FIG. 40A releasing from the ring;

FIG. 42A is a cross-sectional view of another embodiment of a pill having a magnetic switching mechanism in a second configuration; and

FIG. 42B is a cross-sectional view of the pill of FIG. 42A in a first configuration.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The devices and methods disclosed herein generally provide for the controlled delivery of a therapeutic to a targeted location within a body. More particularly, methods and devices are provided for controlling the rate of passage of an orally administered container, such as a pill, through a body, as well as for controlling the delivery of a therapeutic within the pill at a specific location within the body. In some embodiments, various types of devices, generally referred to herein as “catchers,” are provided that can actively catch a pill as it passes through a body. The catcher can hold the pill at a specific location within the body until a predetermined event occurs, such as partial or complete administration of a therapeutic within the pill. The catcher can then release the pill upon command and/or upon the occurrence of the predetermined event to allow the pill to pass out of the body. In other embodiments, various types of pills are provided that can actively engage a catcher and remain engaged with the catcher until a predetermined event occurs. The pill can then release from the catcher upon command and/or upon the occurrence of the predetermined event and pass out of the body. A combination of a catcher and a pill is referred to herein as a pill catcher system.

In general, mechanisms disclosed herein that allow a catcher to capture a pill and/or a pill to engage a catcher can be classified as magnetically based mechanisms and non-magnetically based mechanisms. Catchers having mechanisms for actively engaging and/or capturing a pill will thus generally be referred to herein as active magnetic catchers or active non-magnetic catchers. On the other hand, when a pill is configured to actively engage a catcher, the catcher is referred to as “passive” and such catchers can be classified as passive magnetic catchers and passive non-magnetic catchers. Thus, the catchers disclosed herein can generally be classified into one or more of the following groups: active non-magnetic, passive non-magnetic, active magnetic, and passive magnetic. As with the catchers, the various pill embodiments described herein can also generally be considered passive or active, depending on whether they actively perform a function to engage a catcher. While the embodiments discussed herein are generally organized according to these groups, it will be appreciated that features from certain embodiments or groups of embodiments can be readily applied to other embodiments or groups of embodiments.

Smart Pills

All of the pills disclosed herein are preferably capable of dispensing a therapeutic agent at a specific time and/or location within a patient. Various pills known in the art as “smart pills” can be used with the present invention. These pills often include, for example, microprocessors, wireless radios, battery operated motors, and pumps disposed in their interiors for dispensing a therapeutic at a specific time and/or location. They can generally be triggered to dispense a therapeutic through a pH identification mechanism in the pill and/or through an external trigger. The various mechanisms and other aspects associated with these smart pills are generally not described in this specification apart from any relation they may have to novel mechanisms for engaging a catcher disclosed herein. Exemplary smart pills include the Philips iPill and the Phillips IntelliCap, both available from Philips Research of Eindhoven, The Netherlands.

Anchors/Tethers

In general, the present invention provides catchers that can be anchored at a desired location within a body, and that are configured to actively or passively catch a pill. The catchers disclosed herein can generally be positionable within any of the various lumens of a human or animal gastrointestinal (“G.I.”) tract, including without limitation the esophagus, the stomach, the duodenum, the jejunum, the ileum, and the colon. Preferably, a catcher can be anchored within the body so that its position is maintained regardless of peristalsis and/or other digestive mechanisms. The anchor can be implanted using any surgical technique known in the art, including open surgical procedures and minimally invasive surgical procedures. In some embodiments, an anchor having a tether, sheath, or other connector coupled to a catcher can be utilized to facilitate positioning of the catcher at a desired location. Since a variety of anchors can be utilized with the pill catcher systems described herein, a brief description of some of the various anchors is provided. It will be appreciated by those having ordinary skill in the art that any of the anchors disclosed herein can be utilized in any combination with any of the catcher and pill embodiments, and that various other anchors known in the art can be used.

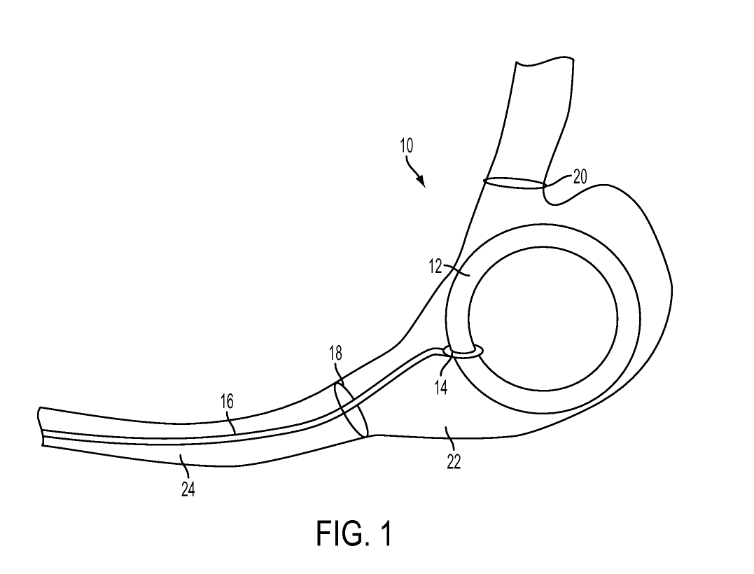

FIG. 1 illustrates one exemplary embodiment of an anchor assembly 10 that can include a major gastric ring 12, a minor gastric ring 14, and a tether 16. The major gastric ring 12 is sized such that it is larger than the pyloric and esophageal sphincters 18, 20 and therefore can be maintained in the stomach 22. The minor gastric ring 14 can be slidably disposed over the major gastric ring 12. The tether 16, which is coupled to the minor gastric ring 12, can extend distally through the pylorus 18 and into the duodenum 24 or beyond. The tether 16 can be coupled to any of a variety of catchers, and/or can itself be configured to catch a pill traveling through the digestive tract. In use, the sliding interface between the major and minor gastric rings 12, 14 allows the minor ring 14 to remain in close proximity to the pylorus 18, regardless of the position and orientation of the major gastric ring 12. Because the major gastric ring 12 is too large to pass through the pylorus 18, the tether 16 remains in a relatively fixed position within the digestive tract, despite the peristaltic forces acting thereon. This advantageously permits pills that are caught by the tether 16 (or by a catcher coupled thereto) to be held at a desired position within the digestive tract. In one embodiment, the minor gastric ring 14 can be omitted, and the tether 16 can be coupled directly to the major gastric ring 12. Further details on gastric ring anchors can be found in International Application No. WO 2008/028108, entitled “AN IMPLANTABLE COIL FOR INSERTION INTO A HOLLOW BODY ORGAN,” the contents of which are incorporated herein in their entirety.

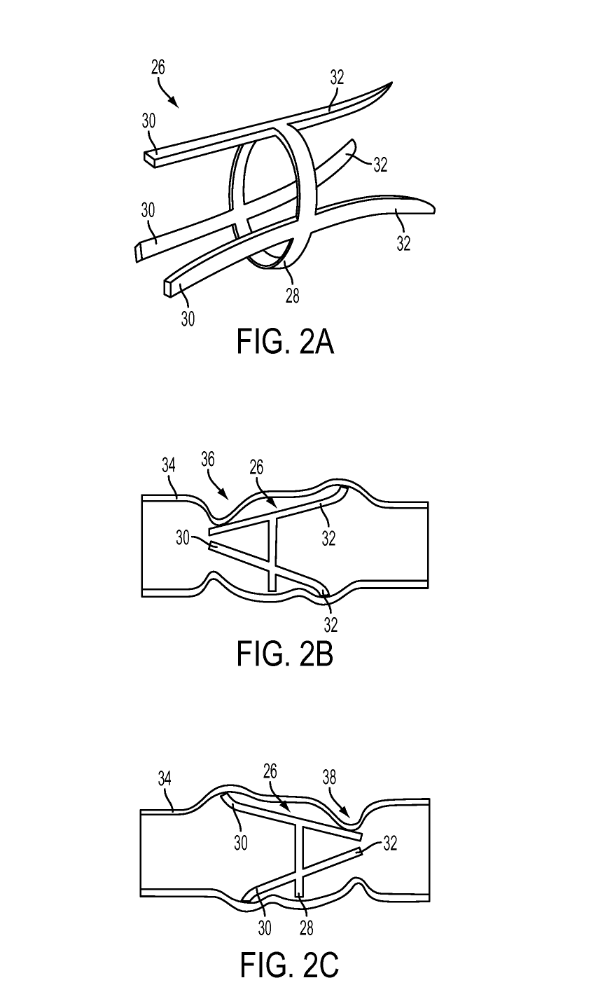

FIGS. 2A-2C illustrate one exemplary embodiment of a peristaltic-resisting anchor 26. The anchor 26 can include a semi-rigid central ring 28 having a plurality of fingers 30, 32 extending proximally and distally therefrom. The anchor 26 can be sized to be positioned within a body lumen 34, as shown in FIG. 2B, to provide a fixed anchoring point to which a tether and/or a catcher can be attached. When positioned in any of the various lumens of the digestive tract, the elongate fingers 30, 32 can provide a peristalsis-resisting function. As shown in FIG. 2B, when peristaltic contraction of the body lumen 34 occurs near the distal end 36 of the anchor 26, the distal fingers 30 can deform inwards towards the central axis of the lumen 34. This causes the proximal fingers 32 to deform outwards into the wall of the lumen 34, resisting movement of the anchor 26 relative thereto. Thereafter, when peristaltic contraction of the body lumen 34 occurs near the proximal end 38 of the anchor 26, as shown in FIG. 2C, the proximal fingers 32 deform inwards. At the same time, the distal fingers 30 deform outwards, again restricting movement of the anchor 26 within the lumen 34. Accordingly, as the peristaltic action of the lumen 34 continuously alternates between the configurations of FIGS. 2B and 2C, the fingers 30, 32 and the central ring 28 of the anchor 26 can flex with the lumen 34 and maintain the anchor 26 at a fixed position relative thereto. In the absence of a peristaltic wave, the fingers 30, 32 can remain in a neutral position in which the force exerted on the body lumen 34 is at a minimum. Accordingly, the anchor 26 can be configured to apply a peristalsis-resisting force only when necessary, thereby avoiding damage to the body lumen 34.

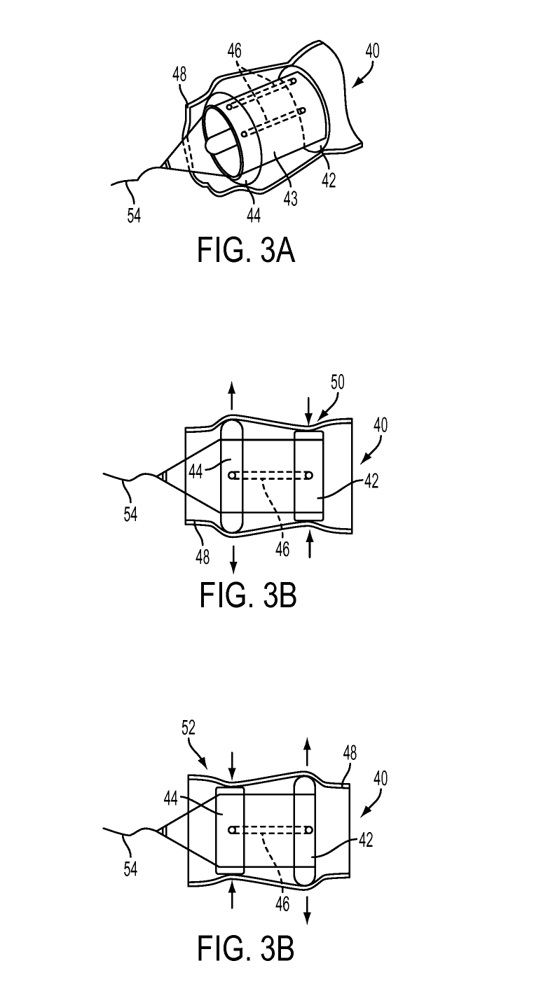

FIGS. 3A-3C illustrate another exemplary embodiment of a peristaltic-resisting anchor 40. As shown, the anchor 40 is generally cylindrical and can include opposed proximal and distal bladders 42, 44 formed on an exterior surface 43 thereof. At least one fluid lumen 46 can maintain fluid communication between the proximal and distal bladders 42, 44. When the anchor 40 is disposed within a peristaltic body lumen 48, alternating inflation and deflation of the bladders 42, 44 can be effective to maintain the anchor 40 at a fixed position relative to the body lumen 48. For example, as shown in FIG. 3B, when peristaltic action of the body lumen 48 causes it to contract near the proximal end 50 of the anchor 40, the proximal bladder 42 is deflated, expelling fluid therefrom into the fluid lumen 46 and, ultimately, into the distal bladder 44. As a result, the distal bladder 44 can inflate against the surrounding body lumen wall 48, holding the anchor 40 firmly in place. Subsequently, when the peristaltic action of the body lumen 48 causes it to contract near the distal end 52 of the anchor 40 (as shown in FIG. 3C), the fluid can be forced out of the distal bladder 44, through the fluid lumen 46, and into the proximal bladder 42. The proximal bladder 42 can then inflate against the lumen wall 48, again holding the anchor 40 in position. Accordingly, the anchor 40 can be maintained at a constant position relative to the body lumen 48, despite the continuous peristaltic action, and can thus provide a fixed anchor point for a tether 54 and/or other catcher. Between peristaltic waves, the proximal and distal bladders 42, 44 can contain similar amounts of fluid and can exert a minimal force on the body lumen 48, thereby preventing erosion or other damage to the body lumen 48 that can occur when a constant force is applied thereto.

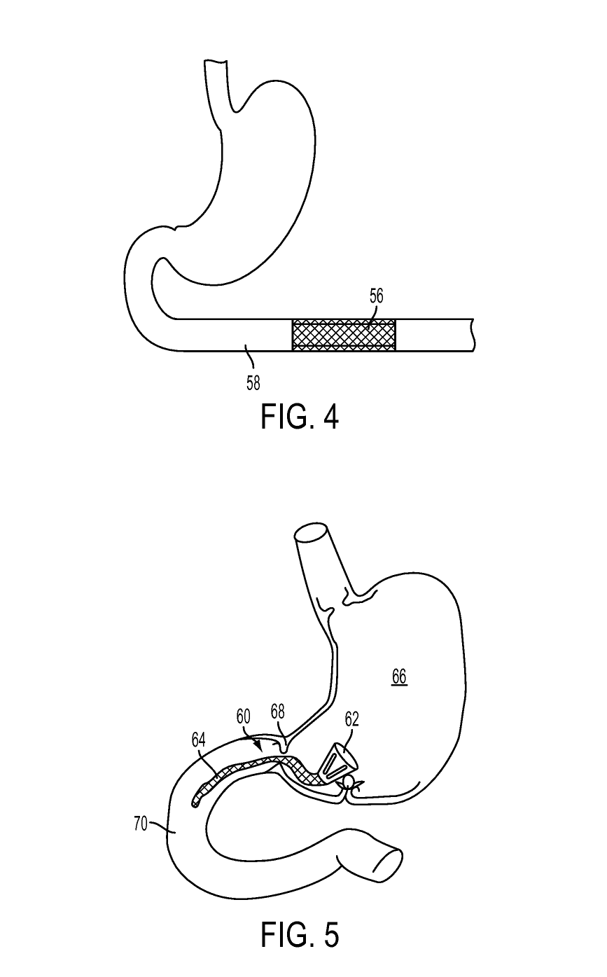

FIG. 4 illustrates another exemplary embodiment of an anchor 56. The anchor 56 can generally be in the form of a cylindrical stent-like structure configured to engage the interior surface of a body lumen 58. The anchor 56 can optionally include various features for gripping the inner wall of the lumen 58, such as hooks, barbs, fingers, etc. The anchor 56 can also be expandable and configured to expand outwardly into contact with the lumen wall 58 when deployed. The anchor 56 can be formed from a variety of materials known in the art, including shape memory alloys such as Nitinol.

FIG. 5 illustrates another exemplary embodiment of an anchor 60. The anchor 60 generally includes a conical proximal funnel 62 coupled to an elongate sleeve 64. The funnel 62 can be positioned within the stomach 66 and sized so as to prevent passage of the funnel 62 through the pylorus 68. The elongate sleeve 64 can extend distally from the funnel 62, through the pylorus 68, and into the duodenum 70 or beyond. The funnel 62 can be sutured in place as shown in FIG. 5, or peristaltic forces can be relied upon to bias the anchor 60 distally, holding the funnel 62 against the pylorus 68 and drawing the sleeve 64 distally. Alternatively, or in addition, the sleeve 64 can include hooks, barbs, and the like for gripping the sidewalls of a lumen in which it is disposed.

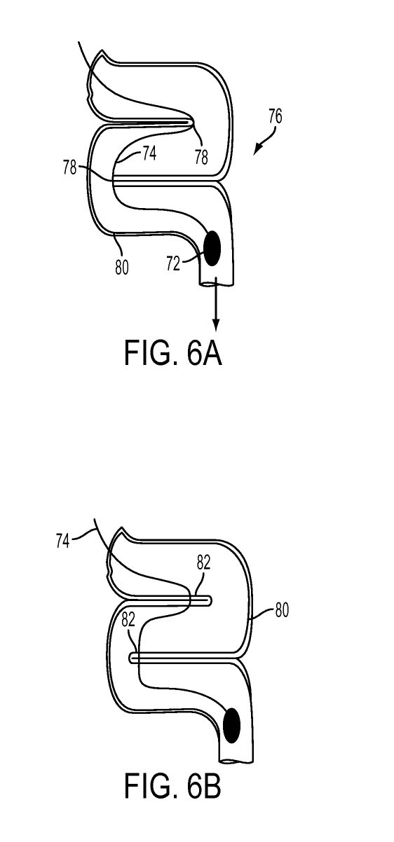

Many of the embodiments disclosed herein can include an elongate tether that extends from an anchor to a catcher. Long-term placement of such tethers within the digestive tract can sometimes have deleterious effects on the various lumens through which they pass. For example, as shown in FIG. 6A, peristaltic forces acting on a pill catching element 72 coupled to the distal end of a tether 74 tend to keep the tether 74 under tension. When positioned in a tortuous segment of the intestine 76, as shown, frictional stress points 78 are formed where the tether 74 rubs against the intestinal sidewall 80. If care is not taken, the tether 74 can erode or abrade the sidewall 80 as shown in FIG. 6B, potentially leading to perforations 82 or other dangerous complications. Accordingly, any of the tethers disclosed herein can include various features for preventing such occurrences. For example, the tethers can include a hydrogel outer membrane or other friction-reducing coating. The tethers can also have a generally flat, ribbon-shaped profile such that the contact stress is spread over a greater surface area of the lumen wall. Similarly, the tethers can optionally be formed of a hollow flexible tubing that will flatten under stress, which can likewise provide a broader contact area. The tethers can also include a friction-reducing coating or sheath, and/or can be notched or otherwise configured to easily lengthen longitudinally under tension. Compliant materials such as silicon can be used to construct the tethers.

Sizes and Materials

A person skilled in the art will appreciate that in any of the pill catcher system, anchor, and/or tether embodiments described herein, the various catchers, pills, anchors, and tethers can have any dimensions as required to accommodate a particular patient. For example, tethers can generally have any length as needed for a particular situation, for example, to reach a specific point within a patient's G.I. tract and/or to accommodate a particularly sized patient such as an infant, child, or adult. For example, an exemplary tether can have any length in a range of about 5 cm to about 7 m, and can have any range of lengths within that range as well. Tethers can also have any diameter as required. Likewise, the various catcher and anchor embodiments can have a size that is dependent on the situation and/or patient. For example, an infant or child patient would likely require an anchor and/or a catcher that is smaller than that required for an adult. Similarly, the size of the pill embodiments can be dependent on what is required of the pill, i.e., how much therapeutic it is required to carry, what kind of catch engagement mechanism it may have, and what size patient will be swallowing it.

The various components that make up the catchers, anchors, and tethers can generally be formed of any biocompatible material known in the art. While many of the embodiments described herein note specific materials that can be utilized therewith, in general the catchers, tethers, and/or anchors can be formed in whole or in part from shape memory alloys such as nitinol, corrosion resistant metals such as 316LVM stainless steel or similar, plastics such as polypropolene, PEEK, Teflon, etc., elastomeric materials such as silicone and urethane, etc.

Active Non-Magnetic Catchers

The following embodiments generally involve an active pill-catcher that does not rely on magnetism to catch, hold, or release the pill. Some of the embodiments disclosed herein include one or more components that are formed partially or completely of a shape memory material. Exemplary shape memory materials include Nitinol, shape memory polymers (SMPs), and other phase change materials. It will be appreciated that the configuration (e.g., the shape, length, diameter, etc.) of such materials can shift between one or more remembered states in response to a stimulus (such as temperature change). For temperature-responsive shape memory materials, the transition temperature is preferably above body temperature (e.g., about 37 degrees C.) and below a temperature that would cause thermal damage (e.g., about 60 degrees C.). More preferably, the transition temperature is in a range of about 45 degrees C. to about 50 degrees C.

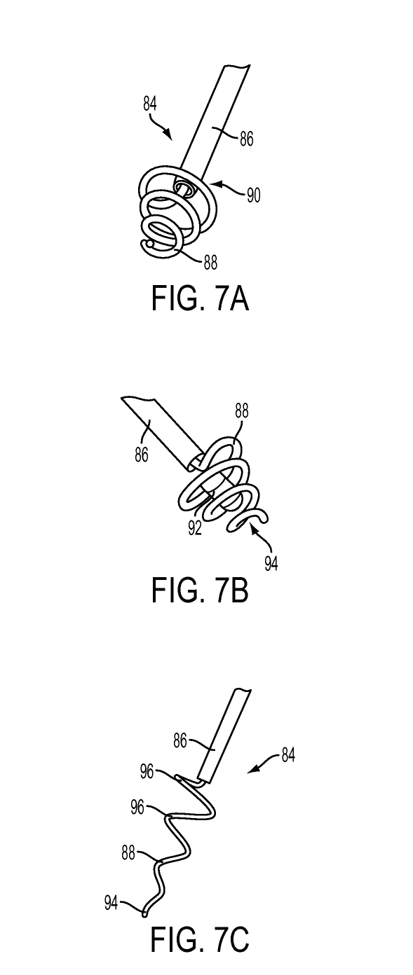

FIGS. 7A-7C illustrate one embodiment of a “pig-tail” catcher 84. The catcher 84 can include an elongate tether 86 with a pig-tail 88 formed on or coupled to a distal end 90 thereof. In the illustrated embodiment, the tether 86 is formed from an elongate electrically-conductive wire having a non-conductive biocompatible coating or sheath disposed therearound. The pig-tail 88 can be a coiled length of shape-memory wire encased in a thermally and electrically non-conductive material.

In use, the proximal end of the tether 86 can be electrically coupled to a transcutaneous-energy-transfer (TET) coil (not shown) and physically coupled to an anchoring device (e.g., the gastric ring anchor of FIG. 1), which can each be disposed within the stomach. The tether 86 can extend through the pylorus such that the pig-tail 88 at the distal end 90 thereof is positioned at a desired location within the patient's digestive tract, for example in the duodenum. When the patient orally ingests a pill 92, the pill 92 advances through the stomach and into the body lumen in which the pig-tail 88 is positioned. The pig-tail 88 can have a first, coiled configuration in which it can act as a conical funnel-shaped cage, guiding the pill 92 towards the distal apex 94 of the pig-tail 88, where it can be held in place and restricted from further movement through the digestive tract. The pill 92 can then be held in place for as long as necessary or desired to release an effective amount of a therapeutic contained within the pill 92. When the desired dosage has been delivered from the pill 92 into the surrounding lumen, or when otherwise desired, an extra-corporeal device can be employed to release the pill 92. In one embodiment, a hand-held unit is provided having a conductive coil and an energy source such as a battery. The unit can be placed in proximity to the implanted TET coil, and a current can be applied to the coil of the hand-held unit. The resulting magnetic field induces a current in the implanted TET coil which can be delivered to the tether 86 and the pig-tail 88 electrically coupled thereto. The induced current can generate resistance heating of the shape-memory pig-tail 88, which can cause the pig-tail 88 to transition to a remembered state, i.e., a second configuration.

As shown in FIG. 7C, the second configuration can be one in which the pig-tail 88 is longitudinally extended, increasing the distance between adjacent coils 96 or even completely straightening the pig-tail 88. No longer restrained by the coil cage 88, the pill 92 can be released from the pig-tail 88 and is free to continue through the digestive tract under the body's natural peristaltic action. When the hand-held unit is deactivated or moved away from the implanted TET coil, current stops flowing through the pig-tail 88, allowing it to cool below its transition temperature and return to the first, coiled configuration shown in FIG. 7A. The catcher 84 is then ready to catch another pill. It will be appreciated that the size and shape of the pig-tail 88 in any of its various states can be selected such that it can catch pills passing through the body lumen but permits chyme, food, and other digestive tract contents to pass by substantially unimpeded. The distal tip 94 of the pig-tail can be hooked back on itself, or can include a ball or other safety feature to make the tip blunt and to prevent the possibility of damage to the body lumen.

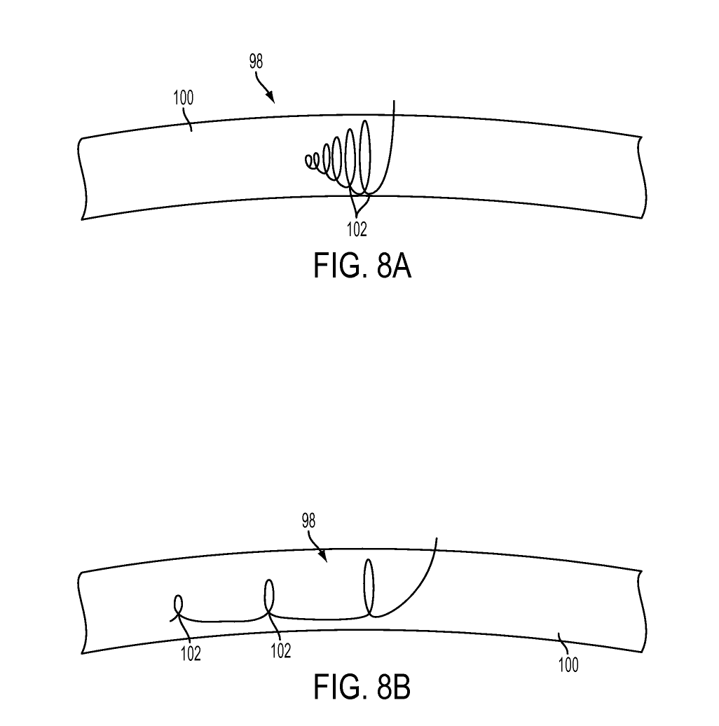

As shown in FIGS. 8A-8B, a pig-tail catcher 98 can also be directly anchored to the digestive tract 100 (e.g., without relying on a separate tether and/or anchor). For example, the catcher 98 can be biased towards a radially-expanded position such that it acts like an expandable stent. In other words, the catcher 98 can expand radially-outward into contact with the surrounding body lumen 100 to hold itself in place. The catcher 98 can include any of a variety of features for further securing itself within a body lumen, such as barbs, hooks, or prongs. The catcher 98 can also be stapled or sutured into place.

As shown in FIG. 8A, the catcher 98 can have a first, substantially conical configuration in which it is effective to catch a pill passing through the digestive tract 100. In this first configuration, the spacing between adjacent coils 102 of the catcher 98 can be large enough to permit chyme to pass through but small enough to restrain a pill. The catcher 98 can also have a second configuration, as shown in FIG. 8B, in which the catcher 98 is at least partially straightened such that the distance between adjacent coils 102 is large enough to permit a pill to pass.

In use, the catcher 98 can be placed in the first configuration to catch a pill administered orally to a patient in which the catcher 98 is disposed. Once the catcher 98 catches the pill and its therapeutic has been dispensed, the catcher 98 can be transitioned to the second configuration, for example in response to a triggering signal, to release the pill. A variety of techniques can be used to transition the catcher 98 between the first and second configurations. In the illustrated embodiment, the catcher 98 can be formed of a shape memory material. At body temperature, the catcher 98 is maintained in the first configuration (e.g., as shown in FIG. 8A). When heat is applied to the catcher 98, for example using microwave energy or inductive heating as described above, the catcher 98 can be transitioned to the second configuration (e.g., as shown in FIG. 8B).

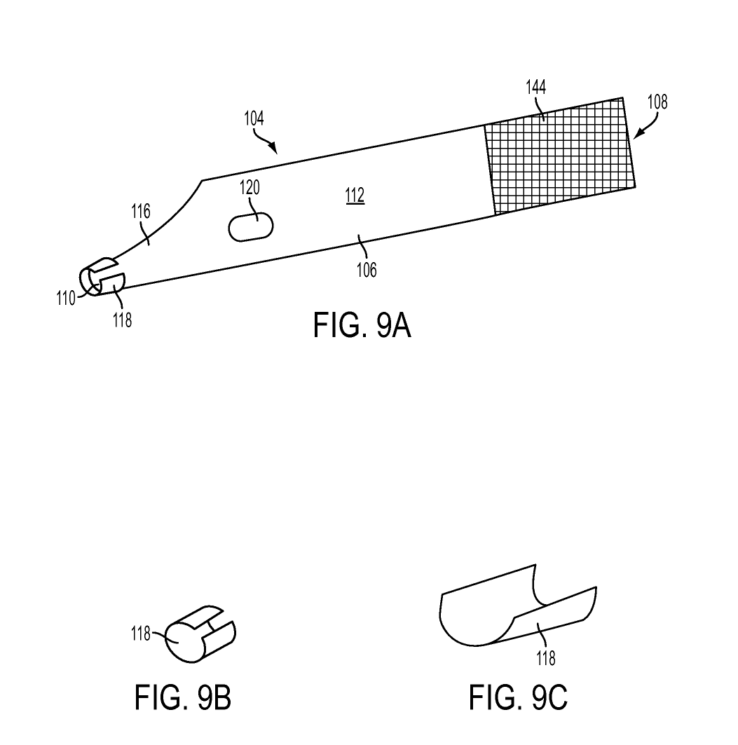

FIGS. 9A-9C illustrate an exemplary embodiment of an endoluminal sleeve catcher 104. The catcher 104 can generally include an elongate tubular sleeve 106 having proximal and distal openings 108, 110 and an inner lumen 112 extending therebetween through which digestive tract contents can pass. In the illustrated embodiment, the sleeve 106 includes an expandable stent-like portion 114 adjacent to the proximal opening 108 that is configured to radially-expand into contact with a surrounding body lumen to hold the sleeve 106 in place relative thereto. The sleeve 106 can be a “barrier” sleeve, meaning it can completely isolate chyme and other contents passing through the inner lumen 112 of the sleeve 106 from the surrounding body lumen. Alternatively, the sleeve 106 can be formed of a mesh or can include one or more apertures to permit nutrient absorption therethrough. A distal portion 116 of the sleeve can be conically-tapered such that material or objects passing through the sleeve 106 are funneled towards the distal opening 110. A C-shaped cylindrical clamp element 118 can be disposed circumferentially around the distal opening 110 and can be selectively actuated to transition the sleeve catcher 104 between a first configuration in which it is effective to capture a pill 120 passing through the sleeve 106, and a second configuration in which it is effective to allow passage of the pill 120 through the sleeve 106. In one embodiment, the clamp element 118 can be formed from a shape memory material having a transition temperature above the body temperature of a patient.

In use, the catcher 104 can be anchored in the digestive tract of a patient such that food, pills, and other objects ingested orally by the patient are passed through the catcher 104. At normal body temperature, the clamp element 118 has a reduced or contracted configuration as shown in FIG. 9B, which prevents pills of sufficient size from passing through the sleeve catcher 104. Once the sleeve catcher 104 is inserted into the patient's digestive tract, appropriately sized pills swallowed by the patient will be unable to pass through the conically-tapered distal end 116 of the sleeve 106, and will therefore be held in a fixed position within the digestive tract. Once the pill has dispensed a desired amount of a therapeutic, the clamp element 118 can be heated above its transition temperature (e.g., using microwave or inductive heating as discussed above), such that it transitions to an enlarged or expanded configuration as shown in FIG. 9C. The clamp element 118 can be sized in this expanded configuration to release the pill, allowing it to pass through the remainder of the patient's digestive tract. Once the pill is released, the clamp element 118 can be allowed to cool back down to body temperature, at which point it returns to the contracted configuration shown in FIG. 9B and becomes ready to catch another pill.

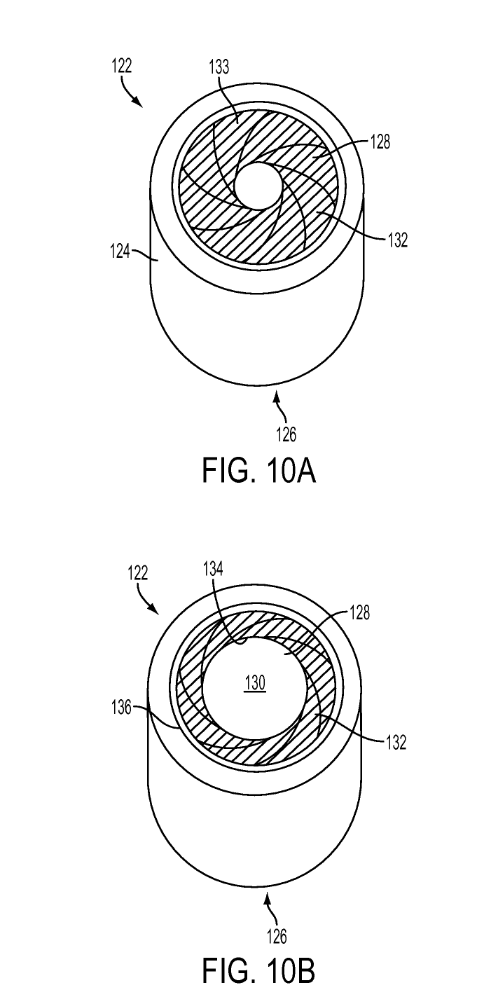

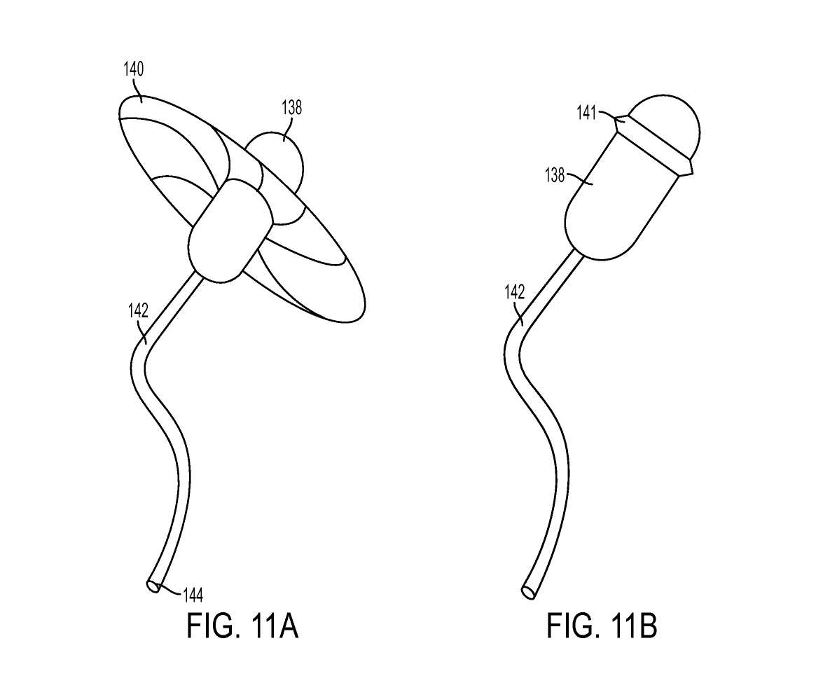

FIGS. 10A-10B illustrate one exemplary embodiment of a “dilating iris” catcher 122. The catcher 122 can include a cylindrical body 124 having proximal and distal openings 126, 128 and an inner lumen 130 extending therebetween. The effective diameter of the distal opening 128 can be varied by actuating (e.g., dilating and/or contracting) an iris diaphragm 132 disposed across the opening 128. In one embodiment, the iris diaphragm 132 can include a plurality of panels 133 that are hingeably mounted to an inner ring 134 at a first end thereof and to an outer ring 136 at a second end thereof. The diaphragm 132 can be dilated by changing the radial position of the inner ring 134 relative to the outer ring 136 such that the angle of the hinged panels 133 relative to the inner and/or outer rings 134, 136 can be adjusted. This can be accomplished by rotationally fixing the outer ring 136 relative to the surrounding body lumen and then rotating the inner ring 134 using a shape memory material or internal motor and worm gear mechanism (not shown).

In use, the catcher 122 can be positioned within the digestive tract of a patient (e.g., anchored directly by barbs, hooks, sutures, clips, staples, expandable mesh, etc., or anchored remotely with a tether). The diameter of the central aperture of the iris diaphragm 132 can be adjusted to regulate the passage of a pill therethrough. For example, the iris diaphragm 132 can be placed in a first, contracted configuration shown in FIG. 10A when it is desired to catch and hold a pill ingested by the patient. Once the desired dose has been delivered or some event has occurred, the iris diaphragm 132 can be placed in the second, dilated configuration shown in FIG. 10B to release the pill and allow it to pass through the remainder of the digestive tract.

Passive Non-Magnetic Catchers

FIGS. 11A-27D illustrate various embodiments that generally involve passive pill-catchers that do not rely on magnetism to catch, hold, or release the pill. Some of the embodiments disclosed herein include one or more dissolvable or bioabsorbable elements or coatings. Any of a variety of biocompatible materials known in the art can be used to form such elements or coatings, including gelatin, polydioxanone (PDS), Vicryl™ (an absorbable synthetic braided suture formed from polyglactin 910 available from Ethicon, Inc., Somerville, N.J.), ProNova™ suture available from Ethicon, Inc. of Somerville, N.J., 316L stainless steel, substantially pure iron, substantially pure zinc, etc. It will be appreciated that the composition of such elements can be selected so as to control the rate at which the elements dissolve, or the conditions under which they will dissolve (e.g., exposure to moisture or liquids, exposure to bile, exposure to a particular temperature or range of temperatures, exposure to a particular pH or range of pHs, and/or any combination thereof). Accordingly, the location within the patient's digestive tract at which the elements or coatings dissolve can also be controlled. For example, a coating that is sensitive to a low pH can be used when it is desired to dissolve the coating in the patient's stomach, and a coating that is bile-sensitive can be used to dissolve the coating in the patient's duodenum. Hydroscopic materials that are configured to expand when exposed to the interior of a digestive tract can also be employed. Elements formed from such materials can be initially compressed into a very small space (e.g., to facilitate swallowing of a pill to which they are coupled) and can then expand to a larger operating size once ingested.

FIGS. 11A-11B illustrate one exemplary embodiment of a pill 138 having an expandable frame 140. The pill 138 can have a first configuration, shown in FIG. 11A, in which it is configured to engage a portion of a patient's digestive tract or a catcher disposed therein. The pill 138 can optionally include a second configuration, shown in FIG. 11B, in which the expandable frame 140 is retracted and/or restrained to allow the pill 138 to be swallowed by a patient or to pass through a catcher disposed within the patient's digestive tract. An extension tube 142 can optionally be provided to allow the pill 138 to release a therapeutic agent at a location remote from the location of the pill 138 itself.

In use, a patient can swallow the pill 138 while it is in the configuration shown in FIG. 11B. The pill 138 can be initially restrained into this configuration by a dissolvable coating and/or a dissolvable ring 141. Once triggered (e.g., by being exposed to body temperature, stomach acids, or some other stimulus), the coating and/or ring 141 can dissolve, allowing the resilient expandable frame 140 to be deployed from the pill 138 such that the pill 138 transitions to the configuration shown in FIG. 11A. In this configuration, the expanded frame 140 prevents the pill 138 from passing through the pylorus, thereby holding the pill 138 in place to achieve targeted delivery of the pill's therapeutic. If the pill 138 includes an extension tube 142, peristaltic digestive forces can draw the extension tube 142 through the pylorus and into the duodenum or beyond, where the pill's active ingredient can be released from the distal end 144 of the tube 142. It will thus be appreciated that the length of the tube 142 can be selected to target a specific site in the digestive tract. For example, if it is desired to administer a drug to a lesion that is 10 cm distal to the pylorus, the extension tube 142 can be trimmed to a length of 10 cm such that when the pill 138 is lodged in the pylorus, the extension tube 142 delivers the drug to the precise location of the lesion.

In embodiments in which the pill 138 engages a catcher disposed within the digestive tract, the extension tube 142 can be omitted such that the pill 138 releases the drug at the location of the catcher, or the length of the extension tube 142 can be selected based on the location of the treatment site relative to the catcher (rather than relative to the pylorus).

The expandable frame 140, or the pill 138 itself, can be configured to dissolve or break apart after sufficient time has elapsed to release the desired amount of the drug. Once the frame 140 and/or pill 138 dissolves and/or breaks apart, it is no longer restrained by the pylorus or catcher, and it is free to pass through the remainder of the patient's digestive tract.

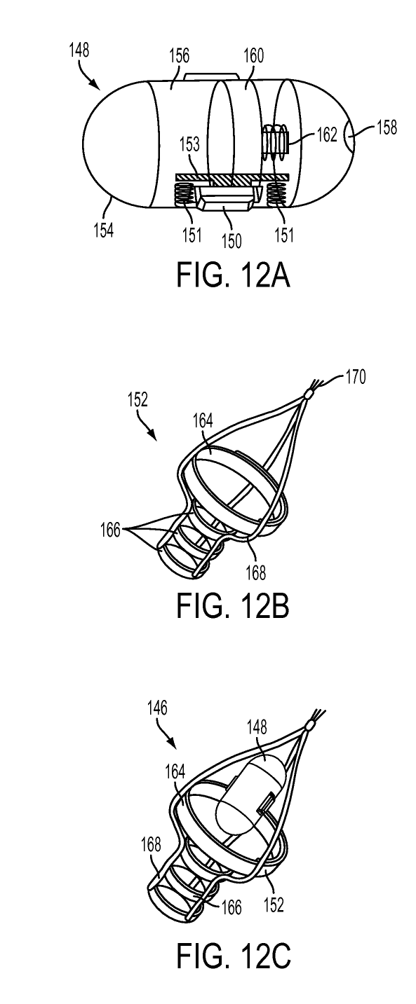

FIGS. 12A-12C illustrate one exemplary embodiment of a pill catching system 146 that can include a pill 148 with deployable wings 150 and an associated catcher 152. As shown in FIG. 12A, the pill 148 can generally include an outer casing 154 defining an internal drug reservoir 156 and one or more wings 150 extending radially from or through the outer casing 154. The pill 148 can have a first configuration (e.g., a configuration in which the wings 150 are deployed) in which the pill 148 is configured to engage a corresponding catcher 152 (e.g., as shown in FIG. 12B) to hold the pill 148 in a fixed position within a patient's digestive tract. The pill 148 can also have a second configuration (e.g., a configuration in which the wings 150 are retracted or dissolved) in which the pill 148 is configured to release from the catcher 152 and/or pass through the digestive tract unimpeded.

In one embodiment, the wings 150 can be passive. For example, the wings 150 can be fixed relative to the outer casing 154 and the pill 148 can be ingested with the wings 150 already deployed (e.g., with the pill 148 in the first configuration). With the wings 150 extended, the pill 148 can be caught by a catcher 152 disposed in the patient's digestive tract. In such embodiments, the wings 150 can be formed from a dissolvable or bioabsorbable material such that they dissolve within the body after a sufficient amount of time has elapsed to release the desired amount of the drug. Once the wings 150 dissolve, the pill 148 is in its second configuration and is free to pass through the catcher 152.

Each wing 150 can also be inwardly-biased (e.g., towards a central axis of the pill 148) by one or more springs 151 positioned between the outer casing 154 of the pill 148 and a support beam 153 to which the wing 150 is mounted. Despite the inward-bias, the wings 150 can be maintained in a deployed position by the drug contained in the reservoir 156 and/or by a piston 160 disposed within the reservoir 156. As the pill 148 releases its therapeutic, the piston 160 can advance through the interior of the pill 148 under the force of a piston bias spring 162. Once the piston 160 advances far enough that it is no longer in contact with the wings 150 (or the support beams to which they are mounted), the inwardly-biased wings 150 are free to retract into the interior of the pill 148, thereby allowing the pill 148 to pass through the catcher 152.

FIG. 12B illustrates one embodiment of a catcher 152 that can be used, for example, with the pill 148. As shown, the catcher 152 can generally include a large-diameter ring 164 and one or more small-diameter rings 166. The large-diameter ring 164 can be sized to substantially conform to the interior wall of a body lumen in which the catcher 152 is disposed, and can be collapsible under the peristaltic forces of the lumen to prevent damage thereto. The small-diameter ring(s) 166 can be coupled to the large-diameter ring 164 by one or more support ribs 168, which in the illustrated embodiment are in the form of elongate struts that are adhered or otherwise coupled to an external surface of the rings 164, 166. The ratio of the diameter of the small-diameter rings 166 to the diameter of the large-diameter ring 164 and the spacing between adjacent rings 164, 166 can be selected to permit chyme, food, and other digestive tract contents to pass through the catcher 152 while preventing the pill 148 from passing through the catcher 152.

The catcher 152, and in particular the support ribs 168, can be coupled via a tether 170 to any of the aforementioned anchors (e.g., a gastric ring anchor). The tether 170 can be a highly flexible cord or spring wound cable, and can be effective to hold the catcher 152 in a fixed position within the patient's digestive tract. Alternatively, or in addition, the catcher 152 can be directly anchored to the digestive tract, for example using staples, clips, barbs, or the like.

In use, the pill 148 is too large in its first configuration to pass through the small-diameter rings 166 of the catcher 152. Accordingly, the pill 148 is held in the catcher 152 until its wings 150 dissolve or are retracted, at which point the pill 148 is free to proceed through the remainder of the patient's digestive tract.

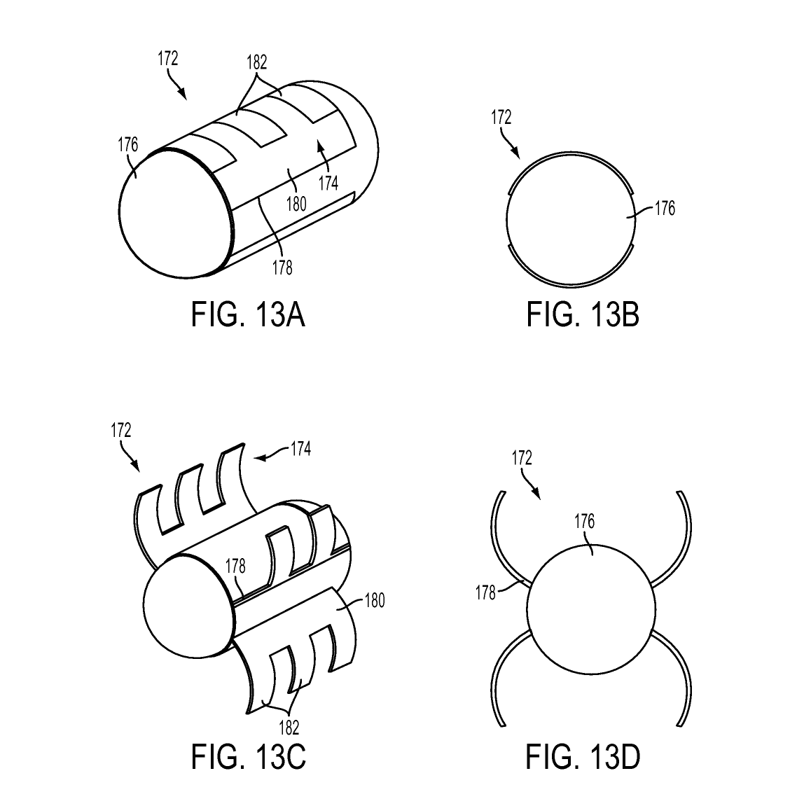

FIGS. 13A-13D illustrate another embodiment of a pill 172 having deployable arms 174. The pill 172 can generally include a vessel 176 containing a drug to be delivered to a site within a patient. One or more arms 174 can be pivotally coupled to an exterior of the vessel 176 at one or more spring hinge joints 178. The arms 174 can generally include an elongate frame 180 having a plurality of prongs or teeth 182 extending transversely therefrom. The pill 172 can have an initial configuration, shown in FIGS. 13A-13B, in which the hinged arms 174 are restrained against the exterior surface of the vessel 176 by a dissolvable coating. As shown in FIG. 13B, the pill 172 can have a generally cylindrical cross-section in this initial configuration such that the pill 172 can be easily ingested by a patient and can pass unimpeded through the patient's digestive tract. Once ingested, the coating can dissolve, allowing the hinged arms 174 to spring outwardly into the configuration shown in FIGS. 13C-13D. In this deployed configuration, the pill 172 can be configured to engage a catcher disposed within the patient and/or to engage a portion of the patient's digestive tract. The hinged arms 174 can be formed from a bioabsorbable material such that they are configured to dissolve after a desired amount of a drug is released from the pill. Once the arms 174 dissolve, the pill 172 can be released from the catcher or lumen in which is it disposed and is again free to pass through the patient's digestive tract.

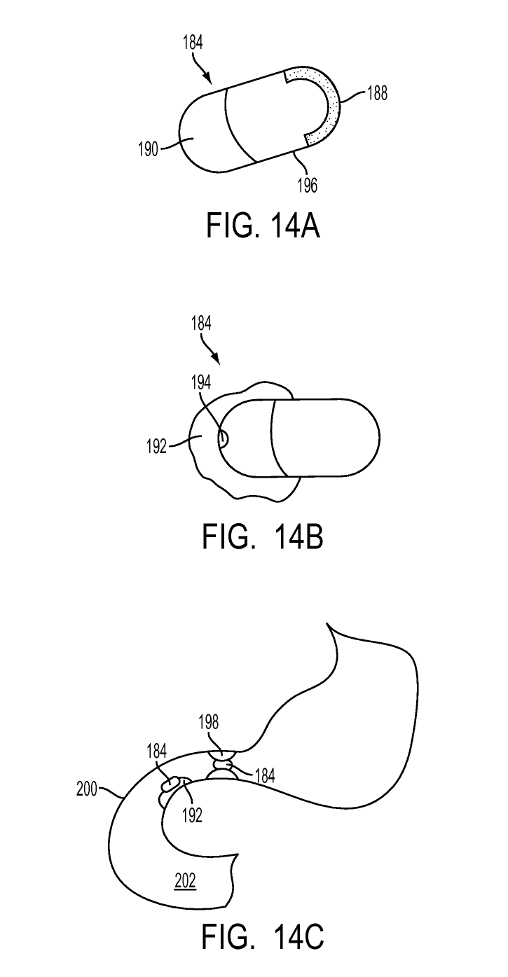

FIGS. 14A-14C illustrate one exemplary embodiment of an adhesive-releasing pill 184. The pill 184 can generally include a vessel 186 containing a drug to be delivered to a site within a patient. The pill 184 can also include a pressure sensor 188, a reservoir 190 of adhesive 192, and an adhesive release mechanism 194. In one embodiment, the release mechanism 194 can include a battery powered pump configured to force the adhesive 192 out of the reservoir 190 and into the patient's digestive tract through one or more pores in the pill's outer shell 196. The adhesive 192 can be any of a variety of biocompatible adhesives known in the art, such as cyanoacrylate, fibrin-based glues, and bacteria-synthesized biosurfactants, and preferably is configured to break down over time when exposed to the interior of a patient's digestive tract.

In use, as shown in FIGS. 14B-14C, the pill 184 can move through the pylorus 198 where the peristaltic pressure exerted on the pill 184 is detected by the pressure sensor 188. The pressure sensor 188 in turn triggers the adhesive release mechanism 194 to deploy the adhesive 192. The deployed adhesive 192 bonds the pill 184 to the inner wall 200 of the body lumen 202 in which the pill 184 is disposed, thereby holding the pill 184 in a fixed position relative thereto while the pill 184 releases the drug. Over time, the adhesive 192 breaks down, eventually releasing the pill 184 from the body lumen 202 and allowing it to pass through the remainder of the patient's digestive tract. In one embodiment, the adhesive 192 can be selected such that its degradation time exceeds the amount of time required to dispense the entire therapeutic within the pill 184.

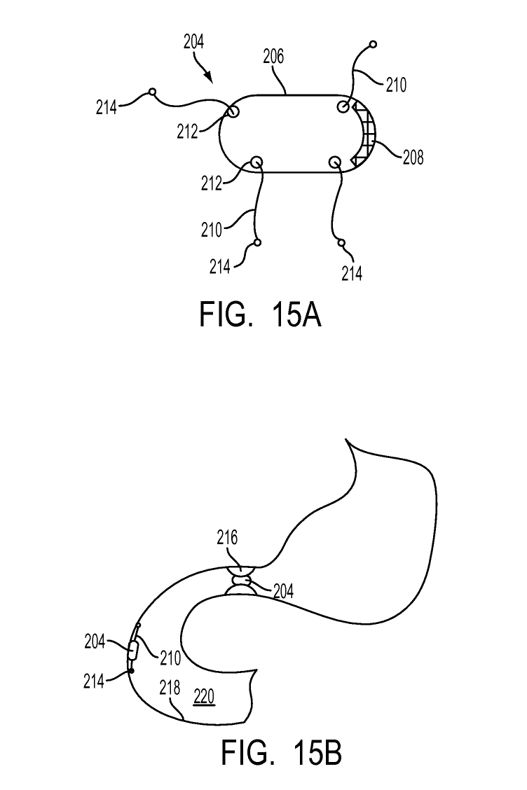

FIGS. 15A-15B illustrate one exemplary embodiment of a leg-deploying pill 204. The pill 204 can generally include a vessel 206 containing a drug to be delivered to a site within a patient. The pill 204 can also include a pressure sensor 208, one or more extendable legs 210, and a leg release mechanism (not shown). In one embodiment, the leg release mechanism can include a battery powered actuator configured to force the legs 210 out of the interior of the pill 204 through one or more openings 212 in the pill's outer shell 206. The legs 210 can generally be in the form of elongate strands having one or more hooks, barbs, or adhesive pads 214 formed thereon or coupled thereto. The legs 210 can be flexible and can be formed from a bioabsorbable material such that they dissolve or break down over time when positioned in a patient's digestive tract. The materials and/or geometry of the legs 210 can be chosen such that the legs 210 break down and fail at a specific location on the leg 210 or at a specific rate.

In use, as shown in FIG. 15B, the pill 204 can move through the pylorus 216 where the peristaltic pressure exerted on the pill 204 is detected by the pressure sensor 208. The pressure sensor 208 can in turn trigger the leg release mechanism to deploy the legs 210. The hooks, barbs, or adhesive pads 214 formed on the legs 210 catch or adhere to the inner wall 218 of the body lumen 220 in which the pill 204 is disposed, thereby holding the pill 204 in a fixed position relative thereto while the pill 204 releases a drug. Over time, the legs 210 break down, eventually releasing the pill 204 from the body lumen 220 and allowing it to pass through the remainder of the patient's digestive tract. In one embodiment, the leg materials are selected such that their degradation time exceeds the amount of time required to release the entire payload of the pill 204.

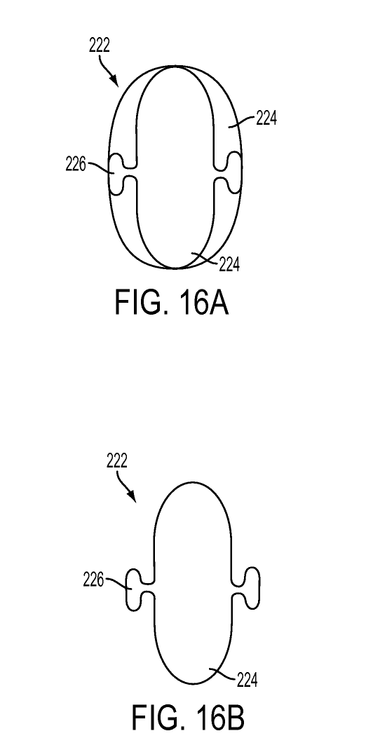

FIGS. 16A-16B illustrate one exemplary embodiment of a pill 222 having a dissolving coating 224 for exposing a catch-engaging member 226. As shown in FIG. 16A, the pill 222 can generally include a vessel 224 containing a drug to be delivered to a site within a patient and at least one catch-engaging member 226 formed on an exterior of the vessel 224. In the illustrated embodiment, the catch-engaging member 226 is an annular projection extending radially outward from an exterior surface of the vessel 224, however a variety of catch-engaging members 226 can be used, including hooks, spikes, prongs, barbs, tabs, ears, fingers, etc. The vessel 224 and the catch-engaging member 226 can be encased in a dissolvable coating 224 such that the pill 222 has an initially smooth exterior. Once ingested, the coating 224 dissolves, exposing the catch-engaging member 226 as shown in FIG. 16B. With the catch-engaging member 226 exposed, the pill 224 can be caught by a catcher disposed within the patient's digestive tract, where it can be held in place to achieve targeted delivery of a drug. The catch-engaging member 226 can be configured to dissolve over time, thereby releasing the pill 222 from the catch and permitting it to pass through the remainder of the patient's digestive tract.

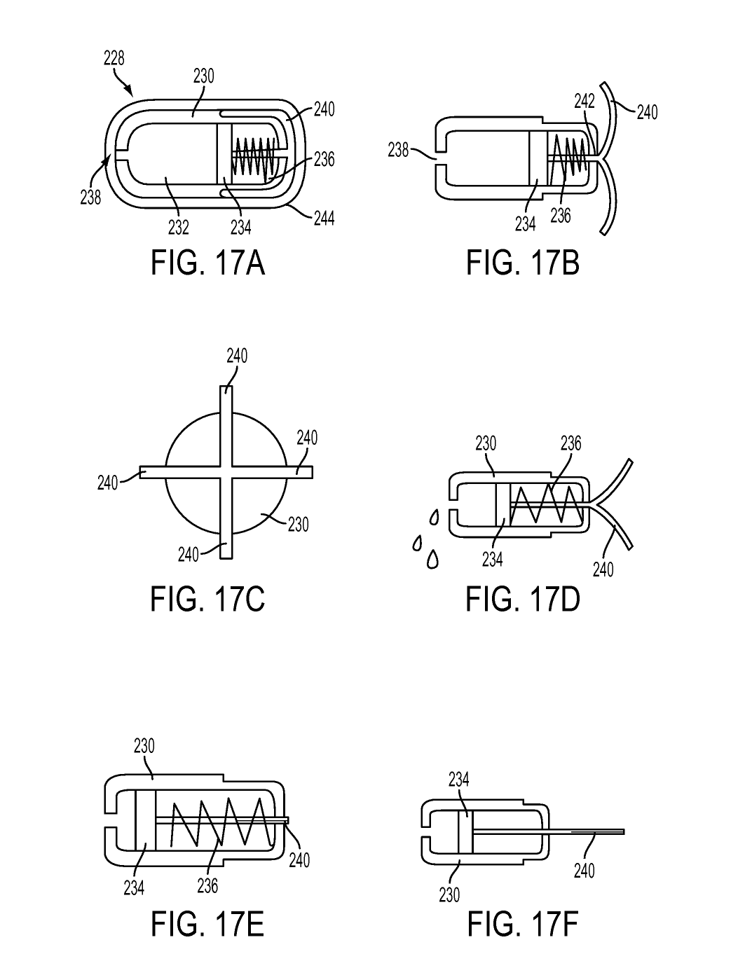

FIGS. 17A-17F illustrate one exemplary embodiment of a pill 228 with deployable and retractable hooks. As shown in FIG. 17A, the pill 228 includes a frame 230 that defines an inner drug reservoir 232. A plunger 234 can be slidably disposed within the reservoir 232 such that the plunger 234 advances therethrough under the force of a bias spring 236, gradually expelling the drug from the reservoir 232 through a distal aperture 238 formed in the frame 230 of the pill 228. One or more flexible hooks 240 can be coupled to the plunger 234 and can extend through a proximal aperture 242 formed in the frame 230 of the pill 228. Any number of hooks 240 can be provided, but in the illustrated embodiment the pill 228 can include four hooks 240 spaced 90 degrees apart from each other, as shown in FIG. 17C. The pill 228 can also include a dissolvable barrier 244 or coating that encases the entire pill 228 and initially restrains the hooks 240 against the exterior surface of the pill frame 230.

In use, the pill 228 shown in FIG. 17A can be ingested by a patient. Shortly thereafter, the coating 244 can dissolve, allowing the previously-restrained hooks 240 to spring outwards as shown in FIG. 17B. In this configuration, the pill 228 can be configured to engage a catch disposed within a patient's digestive tract, or to engage a portion of the digestive tract itself. When the coating 244 dissolves, it can also expose the distal aperture 238 through which the drug contained in the reservoir 232 can be released from the pill 228. As the drug is released through the distal aperture 238, the plunger 234 can slide distally within the pill frame 230 under the force of the bias spring 236. Since the hooks 240 are coupled to the plunger 234, they are retracted or drawn into the frame 230 as the plunger 234 advances, as shown for example in FIG. 17D. Eventually, the drug is fully expelled from the reservoir 232, and the hooks 240 are fully retracted as shown in FIG. 17E. In this configuration, the pill 228 can be configured to release from the catch or from a portion of the patient's digestive tract, allowing the pill 228 to pass.

The hooks 240 can optionally be formed of a shape memory material and can have a remembered state as shown in FIG. 17F. In such cases, immediate release of the pill 228 (e.g., release before the plunger 234 is necessarily fully advanced within the pill 228) can be achieved by heating the hooks 240 as described above (e.g., inductively or using microwave energy).

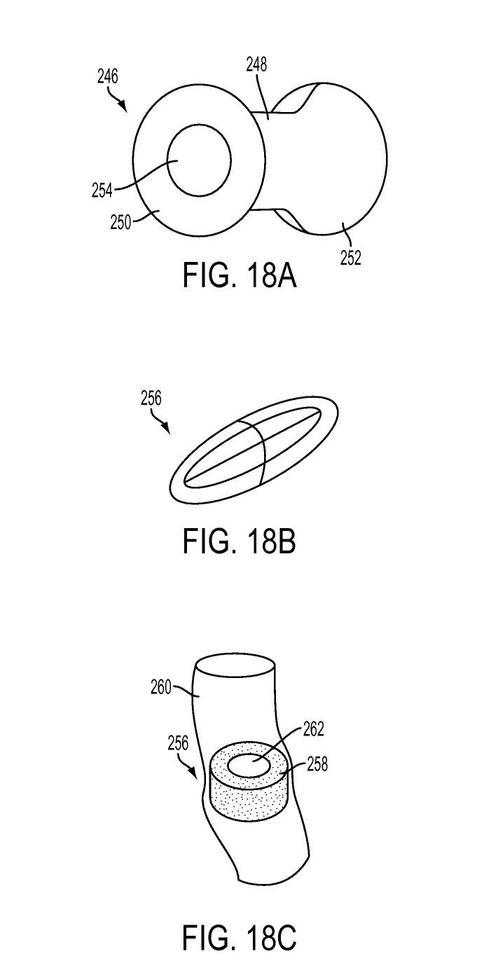

FIG. 18A illustrates one exemplary embodiment of a pill 246 configured to lodge in the pylorus. The pill 246 can generally have an elongate central portion 248 with enlarged proximal and distal flange portions 250, 252. In the illustrated embodiment, a central lumen 254 extends through the entire length of the pill 246 to permit food, chyme, or other stomach contents to pass through the pill 246. In use, the pill 246 can be lodged in the pylorus, either after being orally ingested by the patient or by being placed surgically. While lodged in the pylorus, the pill 246 can gradually release a drug impregnated therein or coated thereon. When the desired release is completed, the pill 246 can be configured to dissolve and/or break up such that it passes through the pylorus and the remainder of the digestive tract. Alternatively, or in addition, the pill 246 can be broken up surgically and allowed to pass or can be removed surgically.

FIGS. 18B-18C illustrate another exemplary embodiment of a “full-lumen” pill 256. The pill 256 can have a reduced-size configuration shown in FIG. 18B in which it is configured to be ingested by a patient. Once exposed to a triggering condition (e.g., moisture, a particular pH, a particular temperature, etc.), an expandable foam portion 258 of the pill 256 can expand to transition the pill 256 to an enlarged-size configuration, as shown in FIG. 18C. When such expansion occurs while the pill 256 is disposed within a body lumen 260, the expanded pill 256 becomes lodged in the body lumen 260 and is thus held at a fixed position relative thereto. While the pill 256 is lodged in place, it can release a drug into the surrounding body lumen 260. The pill 256 can also include a central lumen 262 that extends through the entire length of the pill 256 to allow food, chyme, and other digestive tract contents to pass through the pill 256 while it is lodged in place. When the desired release has been achieved, the pill 256 can be configured to dissolve and/or break apart, thereby dislodging the pill 256 from the body lumen 260 and allowing it to pass through the remainder of the digestive tract.

A number of embodiments disclosed herein involve “hook and loop” type couplings between a pill and a corresponding catcher. It will be appreciated that the component having the hook portion of the coupling and the component having the loop portion of the coupling are interchangeable. Thus, a pill or catcher disclosed herein as having hooks formed thereon can be readily modified to instead have loops formed thereon and vice versa.

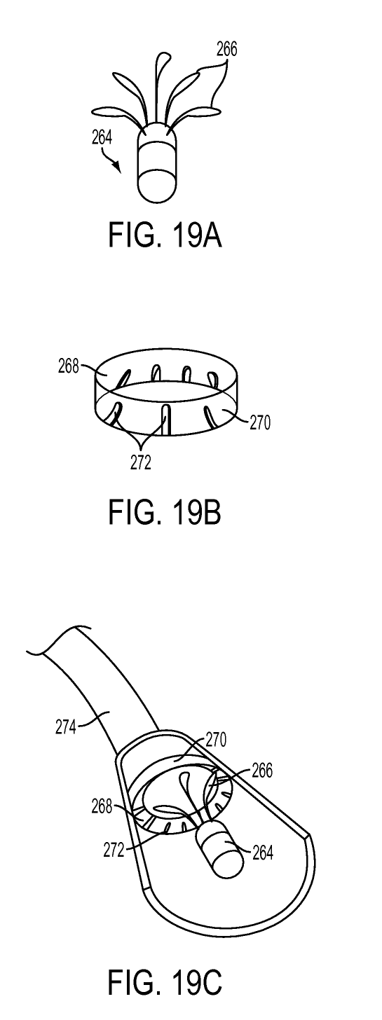

FIGS. 19A-19C illustrate one exemplary embodiment of a pill 264 having bioabsorbable suture loops 266 and one embodiment of a catcher 268 configured to catch such a pill. As shown in FIG. 19A, the pill 264 includes a plurality of suture loops 266 extending therefrom. The loops 266 can be initially restrained against the exterior surface of the pill 264 by a dissolvable coating that is configured to dissolve shortly after the pill 264 is swallowed to release the loops 266. The loops 266 can also be dissolvable, however they can be configured to dissolve slower than the coating.

As shown in FIG. 19B, a barbed ring catcher 268 can be used to catch the pill. The catcher 268 can include an outer support ring 270 with one or more hooks, barbs, or other protrusions 272 extending radially inward therefrom. The ring catcher 268 can be formed from any of a variety of polymeric materials known in the art, including PEEK (polyether ether keytone), and can be sized to conform to the inner diameter of a body lumen 274 in which targeted drug delivery is desired. The ring catcher 268 can be surgically placed within the body lumen 274 and sutured, stapled, T-tagged, or otherwise fixed in place. Alternatively, or in addition, the catcher 268 can be held in place using any of the anchor and/or tether devices disclosed herein.

In use, as shown in FIG. 19C, the pill 264 can be swallowed and can pass through the digestive tract until it encounters the ring catcher 268, at which point the suture loops 266 can be snagged or snared by the barbs or hooks 272 formed on the ring 270. The pill 264 can then be held in place by the suture loops 266 until the desired release is achieved, at which time the suture loops 266 can be configured to dissolve and release the pill 264 from the catcher 268. As noted above, the absorbable properties of the suture loops 266 can be varied to control the amount of time that the pill 264 is held in place. The hooks 272 formed on the catcher 268 can be non-absorbable, and therefore can be reused to catch subsequent pills.

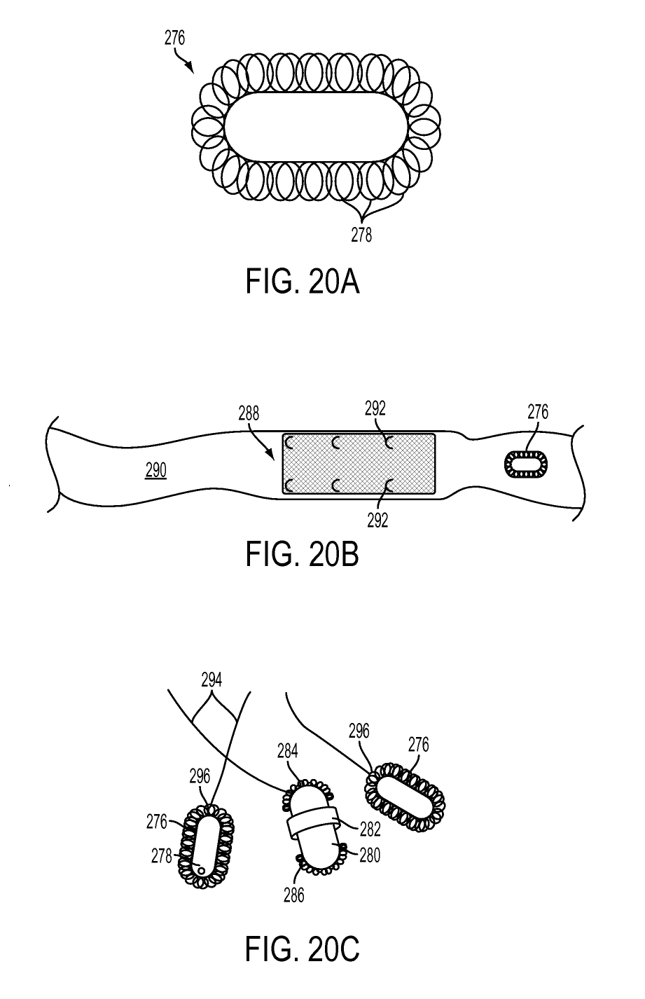

FIGS. 20A-20C illustrate additional exemplary embodiments of pills and associated catchers that employ a “hook and loop” type catching mechanism. As shown in FIG. 20A, a pill 276 can include a plurality of dissolvable suture loops 278 such that substantially the entire exterior surface of the pill 276 is covered with loops 278. The pill can also be partially covered in suture loops. For example, as shown in FIG. 20C, a pill 280 can have a central band 282 of suture loops, a proximal suture loop cap 284, and/or a distal suture loop cap 286. As noted above, the pill can be initially coated with a dissolvable coating that restrains the loops, the coating being dissolvable shortly after the pill is ingested by a patient.

As shown in FIG. 20B, a stent catcher 288 can be placed in a body lumen 290 and it can be configured to catch a pill 276. The stent catcher 288 can be held in a fixed position relative to the body lumen 290 by expanding against an inner wall thereof or by a tether coupled to an anchoring device as described above. The stent catcher 288 can include a plurality of hooks 292 that correspond in size and/or shape to the loops 278 formed on the pill 276. Accordingly, when the pill 276 is ingested by a patient and passes through the stent catcher 292, the suture loops 278 on the pill 276 can be engaged by the catcher hooks 292 to hold the pill 276 in place. The pill 276 can then release a desired dosing of a drug and can then be released when the loops 278 eventually dissolve. The hooks 292 formed on the stent catcher 288 can be non-dissolvable such that the stent 288 can be used to catch a subsequent pill.

“Hook and loop” type pills can also be caught by one or more tethers extending through the patient's digestive tract and having corresponding hook or loop features formed thereon. For example, as shown in FIG. 20C, multiple tethers 294 can be anchored in the stomach and can extend a predetermined distance through the pylorus, each tether 294 having a hook or loop region 296 formed on a distal end thereof. Peristaltic action of the digestive tract can cause an ingested pill 276, 280 to come into contact with one or more of the tethers 294, which can catch the pill. Use of a plurality of tethers 294 can advantageously permit multiple pills to be caught simultaneously.

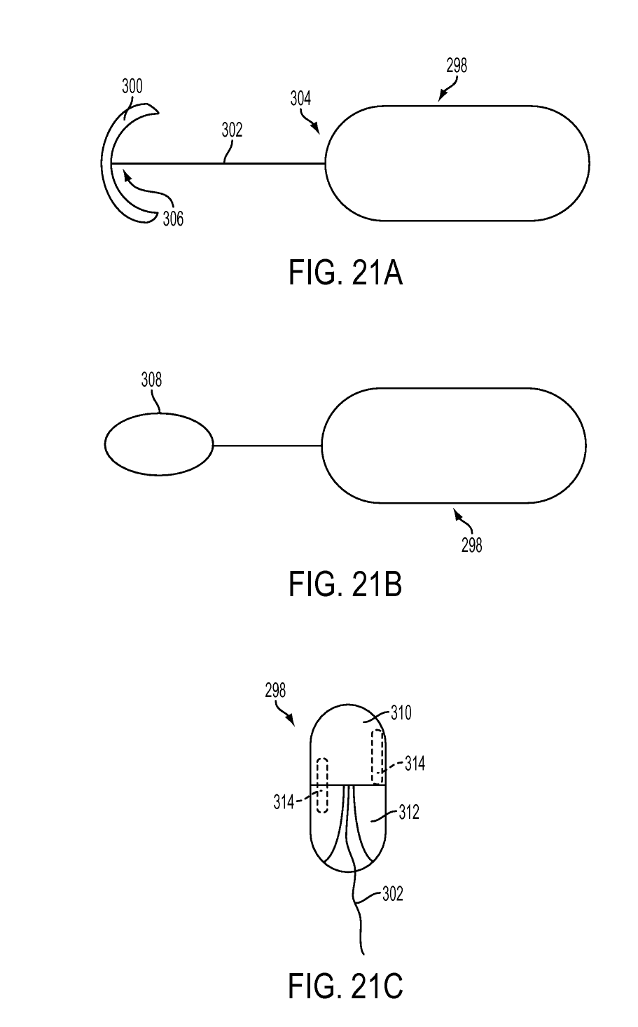

FIG. 21A illustrates one exemplary embodiment of a pill 298 having a dissolvable grappling hook 300 tethered thereto. As shown, the pill 298 can include a tether 302 coupled to a proximal end 304 thereof. One or more curved hook members 300 can be coupled to the proximal end 306 of the tether 302. The tether 302 and/or the hook members 300 can be initially restrained against an exterior of the pill 298 by a dissolvable coating or adhesive configured to dissolve shortly after ingestion by the patient. In use, the pill 298 can be ingested by a patient and the hook members 300 and the tether 302 can be deployed. In one embodiment, the hook members 300 can be formed from a material that expands when exposed to the conditions that exist within the digestive tract. Once the hook members 300 are deployed, the pill 298 passes through the digestive tract until the hook members 300 are caught (e.g., by an anchor or catcher disposed within the patient or by a part of the patient's anatomy). For example, the hook members 300 can be configured to catch into the mucosa of the lower stomach. In such cases, the hook members 300 can optionally be coated or impregnated with a pharmaceutical or nutritional substance which can aid in healing of any damage caused by the hook members catching in the mucosal layer.

Once the hook members 300 are caught, the pill 298 can be held at a fixed distance therefrom by the digestive tract's peristaltic forces, the distance being determined by the length of the tether 302. Thus, when the hook members 300 are caught by the lower stomach or pylorus, the tether 302 can be of sufficient length to permit the pill 298 to pass into the duodenum, where it can achieve targeted release of a drug. The tether 302 and/or the hook members 300 can be formed of a dissolvable material configured to dissolve and/or break apart after an amount of time sufficient to release the desired amount of therapeutic. Once the tether 302 and/or the hook members 300 dissolve, the pill 298 is free to pass through the remainder of the digestive tract unimpeded. The pill 298 can optionally include a plurality of tethers 302, each having its own associated hook member and/or members 300.

As shown in FIG. 21B, the hook members can optionally be replaced with a loop 308 configured to be caught by a corresponding hook formed on an anchor, tether, or catcher disposed within a patient's digestive tract.

As shown in FIG. 21C, the pill 298 can also include an optional release mechanism to actively separate the tether 302 from the pill 298. For example, the pill 298 can include first and second halves 310, 312 rotatably coupled to one another such that rotation of the first half 310 relative to the second half 312 is effective to release the tether 302 from the pill 298. A torsion spring (not shown) can be positioned within the pill 298 to bias the pill halves 310, 312 towards a rotated position in which the tether 302 is released. Actual rotation of the pill 298, however, can be initially restrained by one or more dissolvable locking pins 314. Over time, the locking pins 314 can dissolve, allowing the pill halves 310, 312 to rotate relative to one another under the bias of the torsion spring, thereby releasing the tether 302 from the pill 298 and allowing the pill 298 to pass through the remainder of the digestive tract.

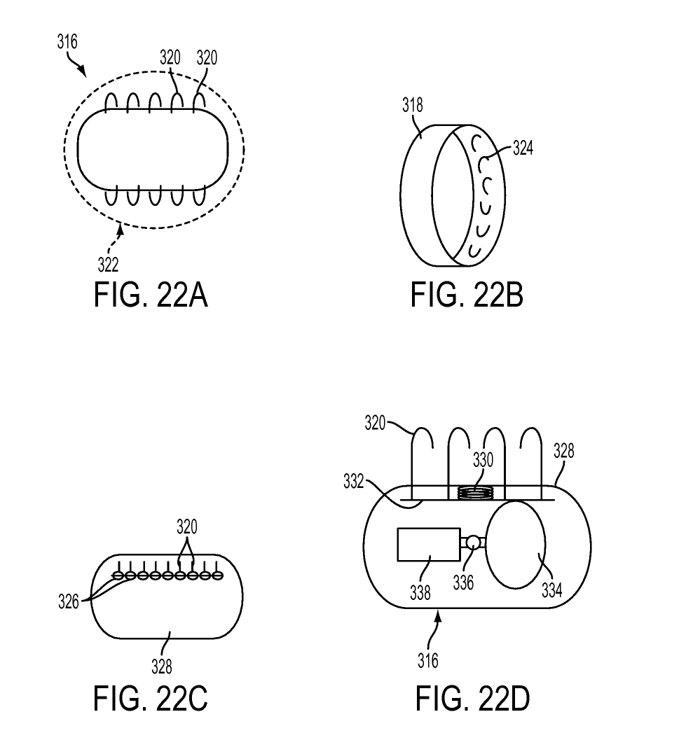

FIGS. 22A-22B illustrate another exemplary embodiment of a pill 316 and associated catcher 318 that employ a “hook and loop” type catching mechanism. As shown in FIG. 22A, the pill 316 includes a plurality of dissolvable hooks 320 extending therefrom. The pill 316 is encased in a dissolvable coating 322 that restrains the hooks 320, the coating 322 being dissolvable shortly after the pill 316 is ingested by a patient.

As shown in FIG. 22B, a ring-shaped catcher 318 can be placed in a body lumen of a patient and can be configured to catch the pill 316. The ring catcher 318 can be held in a fixed position relative to the body lumen by expanding against an inner wall thereof or by a tether coupled to an anchoring device as described above. The ring catcher 318 can also be sutured or stapled directly to the body lumen itself. The ring catcher 318 can include a plurality of loops 324 that correspond in size and shape to the hooks 320 formed on the pill 316. Accordingly, when the pill 316 is ingested by a patient and passes through the ring catcher 318, the suture hooks 320 are engaged by the catcher loops 324 to hold the pill 316 in place. The pill 316 can then release a desired dosing of a drug and can then be released when the hooks 320 eventually dissolve. The loops 324 formed on the ring catcher 318 can be non-dissolvable such that the ring 318 can be used to catch a subsequent pill.

Instead of and/or in addition to the dissolvable coating, the pill 316 can include hooks 320 that are actively deployed and/or retracted. As shown in FIG. 22C-22D, the hooks 320 can be formed from micro wires that are pre-formed to curl into hooks when in their natural state. In a first configuration, shown in FIG. 22C, each wire 320 is disposed within a tubular aperture 326 formed in the outer casing 328 of the pill 316 such that the wire 320 is substantially straight. When the wires 320 are ejected radially outward from the pill 316, as shown in FIG. 22D, their resilient or shape memory properties cause them to form into a hook shape for engagement with corresponding loops 324 formed on a catcher 318. A spring 330 positioned between the outer casing 328 of the pill 316 and an internal support beam 332 to which the wires 320 are mounted can be effective to bias the wires 320 towards a retracted configuration in which they are substantially or completely disposed within the tubular apertures 326. The pill 316 can further include an internal bladder 334 that, when filled with fluid, presses the support beam 332 towards the outer casing 328 of the pill 316, thereby overcoming the force of the bias spring 330 and deploying the hooks 320 from the pill 316. When it is desired to release the pill 316, fluid can be released from the bladder 334, allowing the bias spring 330 to expand longitudinally and draw the hooks 320 back into the pill 316. The filling and/or draining of fluid from the pill's bladder 334 can be controlled by selectively actuating a battery powered pump 336 coupled to a fluid reservoir 338. The actuation of the pump 336 can be triggered by an internal timer or can be based on a measured pH, temperature, pressure, etc.

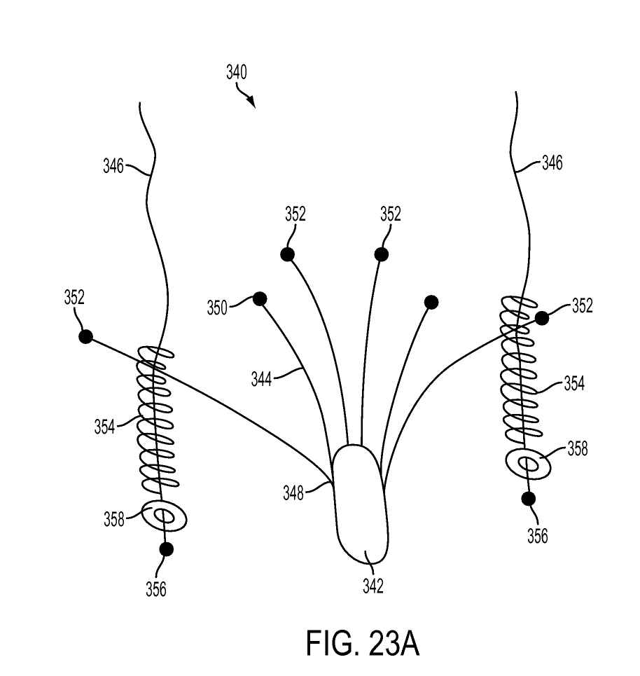

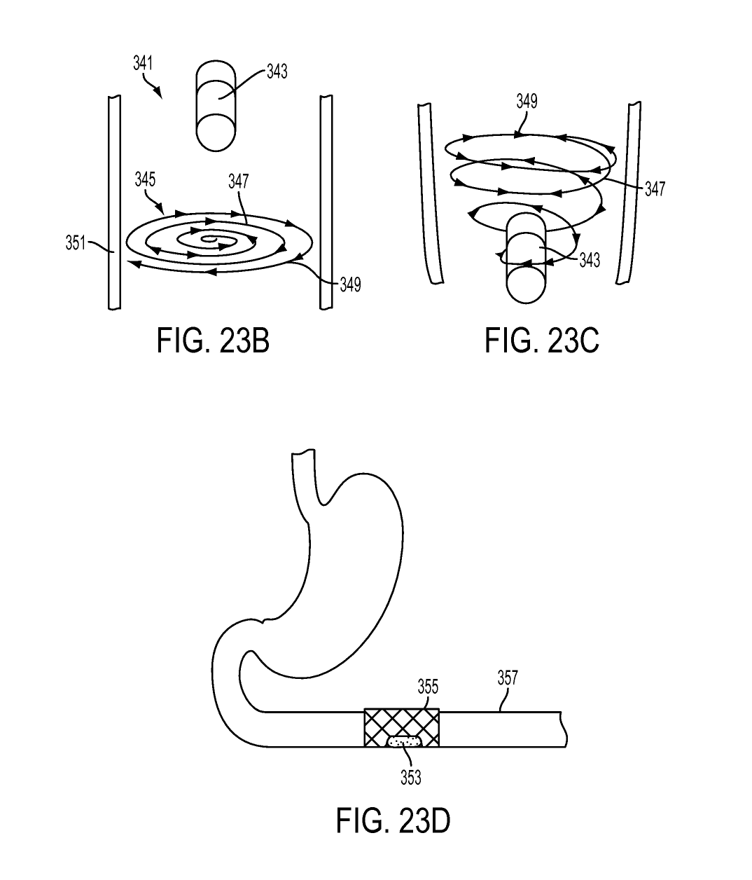

FIG. 23A illustrates one exemplary embodiment of a spring and ball pill catching system 340. As shown, the system generally includes a pill 342 having one or more bioabsorbable sutures 344 and/or strings attached thereto and one or more tethers 346 configured to catch the pill 342.

The sutures 344 can have opposed first and second terminal ends 348, 350, the first terminal end 348 being attached to the pill 342 and the second terminal end 350 being coupled to a sphere, ball, or other increased-diameter object 352. A quick-dissolving coating can be applied to the pill 342 to restrain the sutures 344 against an exterior surface thereof during ingestion, and can dissolve in the stomach to deploy the sutures 344. A spring 354 can be attached to the distal end of each of the tethers 346, for example using a ball 356 and washer 358 as shown. While a generally cylindrical spring 354 is shown in the illustrated embodiment, other spring configurations can also be used, such as conical springs.

In use, the tethers 346 can be anchored as described above such that they extend through at least a portion of a patient's digestive tract. After the pill 342 is ingested by the patient, it can pass through the digestive tract until peristaltic action causes the sutures 344 and/or spheres 352 attached to the pill 342 to tangle with one or more of the springs 354 attached to the tethers 346. As a result, the pill 342 can be held in a fixed position relative to the digestive tract while it delivers a drug thereto. The sutures 344 and/or the increased-diameter object 352 can dissolve after a set period has elapsed that is long enough for the pill 342 to deliver the desired dosing (e.g., one week), at which time the pill 342 is released from the catcher 344 and is free to pass through the remainder of the digestive tract. It will be appreciated that the system 340 can permit multiple pills 342 to be caught simultaneously, particularly when more than one suture 346 or spring 354 is used.

FIGS. 23B-23C illustrate one exemplary embodiment of a coil spring catching system 341. The system 341 can include a pill 343 having a dissolvable mesh coating and a corresponding catcher 345 configured to catch the pill 343. The catcher 345 can include a coiled spring member 347 having one or more barbs 349 or other features formed thereon for engaging the mesh coating of the pill 343. The spring member 347 can be biased such that it radially expands into a surrounding body lumen 351, thereby holding the catcher 345 in place. The catcher 345 can also be sutured or stapled in position. In use, the catcher 345 can be surgically installed in a patient's digestive tract at a site where targeted drug delivery is desired. The patient can then swallow the pill 343, which can subsequently be caught by the barbs 349 of the catcher 345, as shown in FIG. 23C. Once the desired dosage is achieved, the pill's mesh coating can be configured to dissolve, releasing the pill 343 from the catcher 345 and allowing it to pass through the remainder of the patient's digestive tract.

FIG. 23D illustrates one exemplary embodiment of a pill 353 having a deployable stent 355. The stent 355 can be coupled to the exterior of the pill 353, and can be deployed using any of a variety of release mechanisms. In one embodiment, the release mechanism can be a dissolvable coating that initially restrains the stent 355 in a collapsed configuration. Once the coating dissolves, the stent 355 can be free to expand. In another embodiment, an active release mechanism can be used, such as a rip-cord controlled by an on-board motor or actuator. The pill 353 can include a drug payload, and/or the stent 355 can be coated or impregnated with a drug. The pill 353 can also include a camera and a light source to permit visualization by a physician or other individual of a digestive tract in which the pill 353. The pill 353 can also include one or more sensors, such as pH, temperature, or pressure sensors.

In use, the pill 353 can be ingested orally by a patient or delivered to the patient's digestive tract via a catheter. When a site in need of treatment is encountered (e.g., visualized by a physician using the camera or identified by particular pH sensor reading), the pill 353 can be actuated to deploy the stent 355 into contact with the surrounding body lumen 357, thereby restricting further movement of the pill 353 therethrough. The pill's therapeutic can then be released to achieve concentrated, targeted treatment of the site. While the stent 355 is deployed, the pill 353 can be pressed against a sidewall of the body lumen 357, allowing chyme, food, and other digestive tract contents to pass through the central lumen of the stent 355 substantially unimpeded. Once the treatment is completed, the stent 355 can then be configured to dissolve or break up to allow the pill 353 to pass through the remainder of the patient's digestive tract.

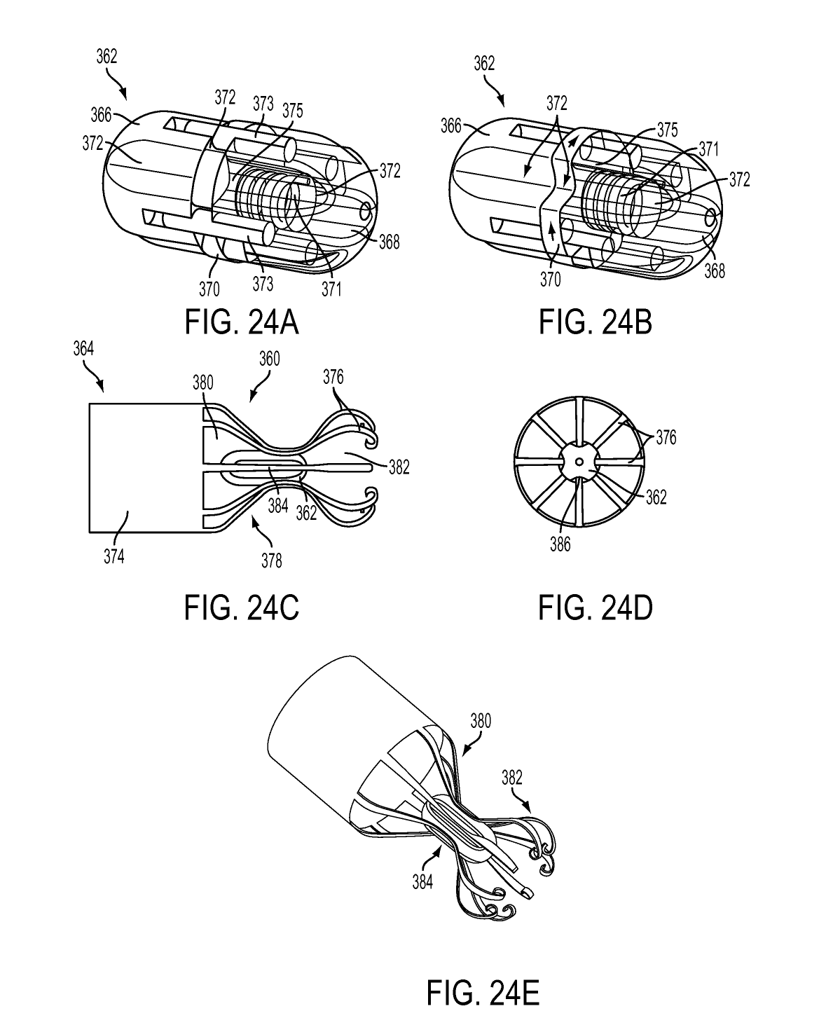

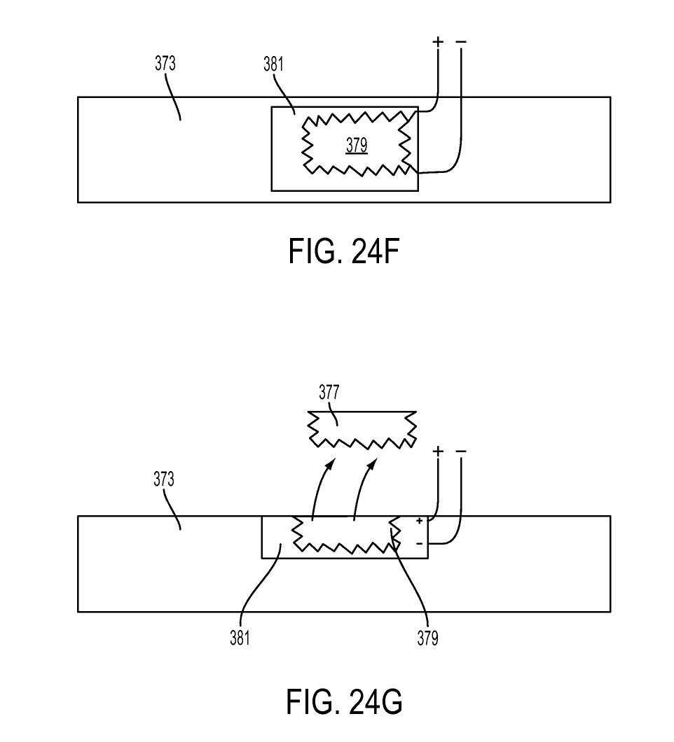

FIGS. 24A-24E illustrate one exemplary embodiment of a pill catching system 360 that includes a pill 362 having a rotatable locking mechanism and an associated catcher 364. The pill 362 can generally include first and second halves 366, 368 separated by a rotatable keyed disk member 370. The first and second halves 366, 368 and the disk member 370 each have corresponding grooves or channels 372 formed in an exterior sidewall thereof. The pill 362 is assembled such that each of the channels 372 in the first pill half 366 are aligned with a corresponding channel 372 in the second pill half 368. In a first configuration, shown in FIG. 24A, the disk member 370 can be positioned such that the channels 372 formed therein are offset from the channels 372 formed in the first and second halves 366, 368. In this configuration, the pill 362 is configured to engage a catcher 364 disposed within a patient's digestive tract, as explained below. The pill 362 also has a second configuration, shown in FIG. 24B, in which the disk member 370 can be rotated such that the channels 372 formed therein are aligned with the channels 372 in the first and second halves 366, 368, thereby forming continuous channels 372 that extend the entire length of the pill 362. In this configuration, the pill 362 can be configured to release from the catcher 364.

The disk member 370 can be selectively rotated in a variety of ways. In the illustrated embodiment, the disk member 370 can be biased by a spring 371 towards the position shown in FIG. 24B, but can be restrained to the position shown in FIG. 24A by one or more dissolvable locking pins 373. The bias spring 371 can have a first end that is fixed relative to the pill 362 and a second, opposite end that engages a notch 375 formed in the disk member 370 such that rotation of the disk member 370 towards the position shown in FIG. 24A winds the spring 371. The locking pins 373 can be configured to dissolve or otherwise break apart as explained below, releasing the disk member 370 and allowing it to rotate under the force of the unwinding bias spring 371 to the position shown in FIG. 24B. In another exemplary embodiment, the pill 362 can include a battery-operated motor that is configured to rotate the disk member 370.

As shown in FIG. 24C, the system 360 can also include a catcher 364 that can be anchored and/or tethered within a patient's digestive tract as described above. The catcher 364 can include a generally cylindrical portion 374 sized to conform to an inner sidewall of a body lumen in which the catcher 364 is disposed. A plurality of curved and/or tapered fingers 376 can extend distally from the cylindrical portion 374, collectively defining an hour-glass shaped cage 378. The fingers 376 can be sized to correspond to the channels 372 formed in the pill 362 and the distal tips thereof can be curved back on themselves to form a blunt leading end, thereby avoiding inadvertent damage to the body lumen in which the catcher 364 is placed. As shown, the finger cage 378 is defined by funnel-shaped proximal and distal ends 380, 382 joined by a reduced-diameter central portion 384, however the distal funnel-shaped portion 382 of the cage 378 can optionally be omitted. As shown in FIGS. 24C-24D, the fingers 376 can be positioned such that the reduced-diameter central portion 384 of the cage 378 defines an opening 386 having a cross-section substantially identical to and/or slightly larger than the maximum cross-section of the pill 362 when configured as shown in FIG. 24B. Accordingly, the pill 362 can pass through the opening 386 when configured as shown in FIG. 24B, but cannot pass through the opening 386 when configured as shown in FIG. 24A.

In use, the pill 362 can be ingested by a patient while in the configuration shown in FIG. 24A. The pill 362 can then travel through the patient's digestive tract until it encounters the catcher 364, where it is guided by the proximal funnel-shaped portion 380 of the cage 378 towards the reduced-diameter central portion 384. Since the disk member 370 is rotated such that it obstructs the channels 372 formed in the pill 362, the pill 362 is unable to pass through the central portion 384 of the cage 378 and becomes lodged in the catcher 364. The pill 362 can then remain in a fixed position relative to the body lumen, gradually releasing a drug. Once the desired dosing has been achieved, rotation of the disk member 370 can be triggered, thereby transitioning the pill 362 to the configuration shown in FIG. 24B and allowing it to pass through the catcher 364 and the remainder of the patient's digestive tract, as shown in FIG. 24E. It will be appreciated that chyme and other contents of the body lumen can continue to pass through the gaps between the fingers 376 even when a pill 362 is captured within the catcher 364.

There are a variety of ways in which the locking pins 373 can be dissolved or broken apart to trigger rotation of the disk member 370. In one embodiment, the locking pins 373 can be positioned on an exterior of the pill 362 and can be configured to dissolve on a time release basis once exposed to the interior of a digestive tract. In another embodiment, the locking pins 373 can be positioned within the pill 362 and the pill 362 can include a piston (not shown) disposed therein that advances through the pill 362 as a drug is released. As the piston advances, it can draw acids or other fluids present in the digestive tract into the interior of the pill 362, exposing the locking pins 373 to the fluids and allowing them to dissolve. Accordingly, the pill 362 can be configured to release from the catcher 364 only when a desired amount of its therapeutic has been released.

In yet another embodiment, as shown in FIGS. 24F-24G, the locking pins 373 can be formed from a dissolvable material and can include a thin protective coating having a removable portion 377. In use, the removable portion 377 of the coating can be removed to open a window 379 and expose the dissolvable interior of the locking pins 373 to the digestive tract, thereby allowing the pins 373 to dissolve. The opening of the window 379 can be triggered by an electrical charge, which in one embodiment can be generated by an onboard battery when a timer elapses. The charge can also be generated when a piston that forces a drug out of the pill advances into contact with a conductive plate. The electrical charge can be applied to the perimeter 381 of the removable portion 377 which can cause localized melting of the protective coating and separation of the removable portion 377. The electrical charge can also be used to ignite a micro blasting cap to break the locking pins 373, or can be used to trigger a chemical reaction that causes the locking pins 373 to break apart or dissolve.

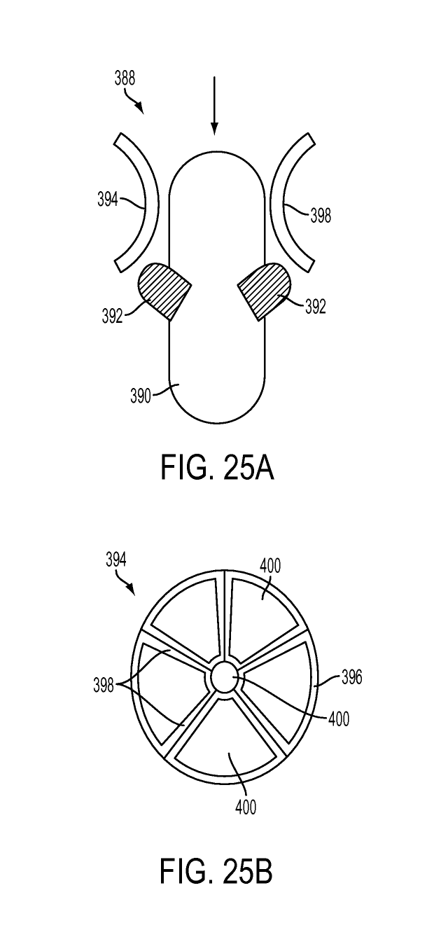

FIGS. 25A-25B illustrate one exemplary embodiment of a pill catching system 388 that can include a pill 390 having an absorbable rib 392 and a corresponding catcher 394. As shown in FIG. 25A, the pill 390 includes an annular rib 392 formed on or coupled to an exterior surface of the pill 390. The rib 392 can extend radially outward from the pill 390, effectively increasing the pill's maximum diameter. The rib 392 can be formed of a bioabsorbable material that is configured to dissolve or break apart after a predetermined time period elapses, which can be selected based on a desired dosing time.

The catcher 394 can be anchored and/or tethered in place as described above. For example, the catcher 394 can be tethered to a gastric ring anchor, can be sutured into position with a body lumen, and/or can be self-expanding. As shown in FIG. 25B, the catcher 394 can include a generally cylindrical portion 396 sized to conform to an inner sidewall of a body lumen in which targeted drug delivery is desired. The catcher 394 also includes a plurality of fingers 398 that extend radially-inward from the cylindrical portion 396 to define a plurality of cross-sectional openings 400 through the catcher 394. The openings 400 can be sized such that the pill 390 is unable to pass though any opening 400 until the rib 392 dissolves and/or breaks apart.

In use, the pill 390 is ingested by a patient in a first configuration in which an absorbable rib 392 is formed thereon and/or coupled thereto. In this first configuration, the pill 390 is configured to engage a catcher 394 disposed within the patient's digestive tract. The pill 390 can proceed through the digestive tract until it encounters the catcher 394, where it becomes lodged in place. Once the desired dosing is achieved, the rib 392 can dissolve and/or break apart, allowing the pill 390 to pass through one of the openings 400 in the catcher 394 and proceed through the remainder of the patient's digestive tract. It will be appreciated that chyme and other digestive tract contents remain free to pass through the openings 400 in the catcher 394, even when a pill 390 is lodged in one of the openings 400.

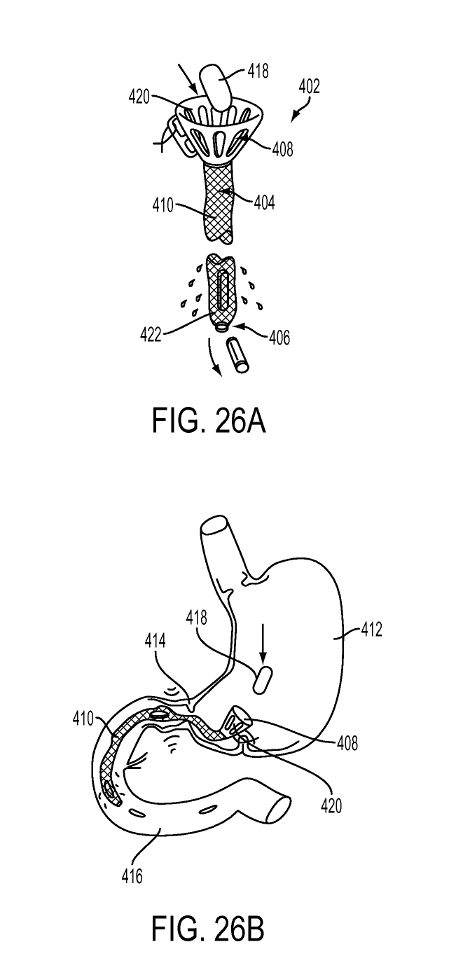

FIGS. 26A-26B illustrate one exemplary embodiment of pill catching system 402 that can include a sock anchor 404 similar to the anchor of FIG. 5 and a distal ring stopper 406. As shown in FIG. 26A, the anchor 404 can include a conical proximal funnel 408 coupled to an elongate sleeve 410. As shown in FIG. 26B, the funnel 408 can be positioned within the stomach 412 and sized so as to prevent passage of the funnel 408 through the pylorus 414. The elongate sleeve 410 can extend distally from the funnel 408, through the pylorus 414, and into the duodenum 416 or beyond. In one embodiment, peristaltic forces can be relied upon to bias the anchor 404 distally, holding the funnel 408 against the pylorus 414 and drawing the sleeve 410 distally. The funnel 408 can also be sutured in place, proximal to the pylorus 414, as shown in FIG. 26B, in which case the churning action of the stomach 412 can be relied upon to guide a pill 418 into the funnel 408 before a cleanout wave through the pylorus 414 occurs. The funnel 408 can include one or more longitudinal slots 420 formed therein and sized to permit food and other stomach contents to escape the funnel 408, while preventing pills 418 from doing so.

The sleeve 410 can include hooks, barbs, and the like for gripping the sidewalls of a lumen in which it is disposed, and can be formed of a mesh material that allows food, chyme, and other digestive tract contents to pass through the sleeve 410. The distal end 422 of the sleeve 410 can be coupled to a ring stopper 406 having a diameter that is smaller than the diameter of the body lumen in which the sleeve 410 is disposed.

In use, a pill 418 can be ingested by a patient in a first, enlarged-diameter configuration. The pill 418 can then enter the funnel portion 408 of the sock anchor 404 and can be guided distally through the sleeve 410 by peristalsis. Before the pill 418 is able to exit the sleeve 410, it can become lodged in the ring stopper 406, which can have a diameter less than that of the pill 418 in the first, enlarged-diameter configuration. Thus, in the first configuration, the pill 418 is configured to engage a catcher 404 disposed within a patient's digestive tract. As the pill 418 releases its therapeutic and slowly erodes over time, it gradually transitions to a second, reduced-diameter configuration in which it is configured to release from the catcher 404. In other words, the pill's diameter can gradually decrease until it is small enough to pass through the ring stopper 406. The rate at which the material from which the pill 418 is formed degrades in the body can be selected to achieve the desired dosing.

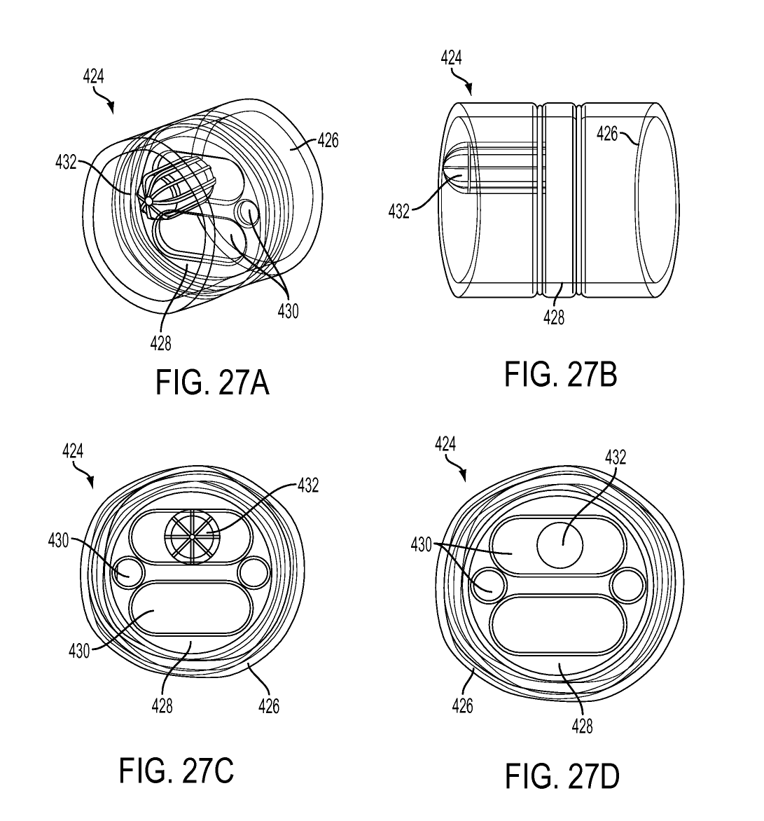

FIGS. 27A-27D illustrate one exemplary embodiment of a slotted tube catcher 424. The catcher 424 can generally include a cylindrical tube 426 configured to be anchored within a body lumen of a patient, for example the duodenum. The catcher 424 can be anchored, tethered, and/or otherwise maintained in position within a patient's digestive tract using any of the techniques described above. The cylindrical tube 426 can include an interior baffle 428 having a plurality of openings 430 formed therein. The openings 430 are sized such that a pill 432 having a first, enlarged-diameter configuration (e.g., a pill as shown in FIGS. 27A-27C) is unable to pass through the openings 430 and is therefore captured by the catcher 424. As the pill 432 releases its therapeutic and gradually degrades over time, the diameter of the pill 432 decreases (e.g., as shown in FIG. 27D) until it is eventually small enough to pass through one of the openings 430 in the baffle 428 and proceed through the remainder of the patient's digestive tract. While the pill 432 is lodged in the catcher 424, food, chyme, and other body lumen contents remain free to pass through the various openings 430 in the baffle 428. The cylindrical tube 426 and the baffle 428 can be flexible and/or collapsible to permit peristaltic waves to pass the catcher 424 without causing erosion or other damage to the surrounding body lumen. For example, the baffle 428 can be configured to fold in half or lean when peristaltic forces are applied thereto.

Active Magnetic Catcher

As noted above, in some embodiments, magnetic mechanisms can be used to facilitate engagement between pills and catchers. The following embodiments generally involve an active catcher that relies on magnetism to catch, hold, and/or release a pill.

There are various ways in which to use magnets to accomplish engagement as will be described below. In some embodiments, whether a magnet is disposed on or within a pill or a catcher, it is desirable to switch the magnet between an “on” configuration and/or a position in which it is attractive to ferromagnetic materials, and an “off” configuration and/or a position in which it is not attractive to ferromagnetic materials. This on and off switching can be utilized in some of the pill catcher system embodiments described herein, and thus the general principle of using a magnetic base for switching will first be described.

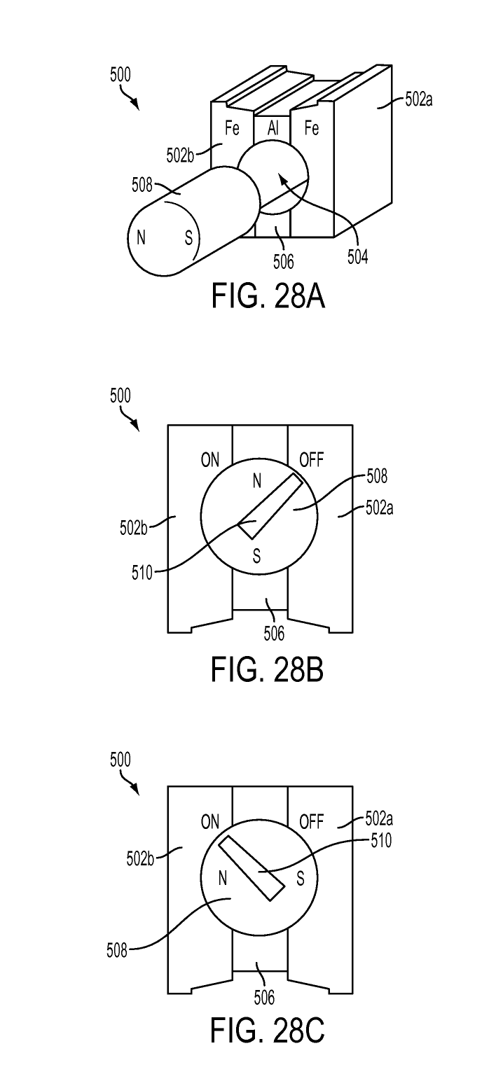

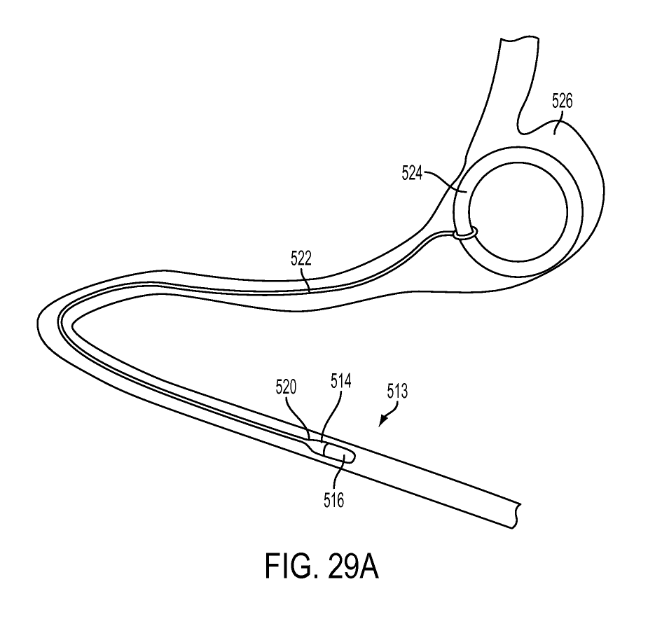

As shown in FIGS. 28A-28C, a magnetic base 500 can generally be made from two blocks 502a, 502b of ferromagnetic material, such as iron, with a round cavity 504 bored through its center. The two blocks 502a, 502b can be joined together with a non-ferrous material 506 such as brass or aluminum. A round permanent magnet 508 can be inserted into the cavity 504, and a handle 510 or other engagement mechanism can be attached to the magnet 508 to allow easy rotation thereof within the cavity 504. Rotation of the magnet 508 can change the direction of its magnetic field so that it is either directed into the two ferromagnetic blocks 502a, 502b, where the ferromagnetic material acts to prevent the magnetic field from extending outside of the base 500 (i.e., the base 500 is in the off position as shown in FIG. 28B), or is directed through the non-ferrous material 506 between the two blocks 502a, 502b so that it extends outside of the base 500 (i.e., the base 500 is in the on position as shown in FIG. 28C). In the on position, the magnetic field of the magnet 508 can effectively pass across an air gap where it can be made to do work if the gap is bridged with another piece of ferromagnetic material. Once the gap is bridged, the ferromagnetic material can become part of the magnetic field's circuit and will be attracted with the full strength of the magnet 508.

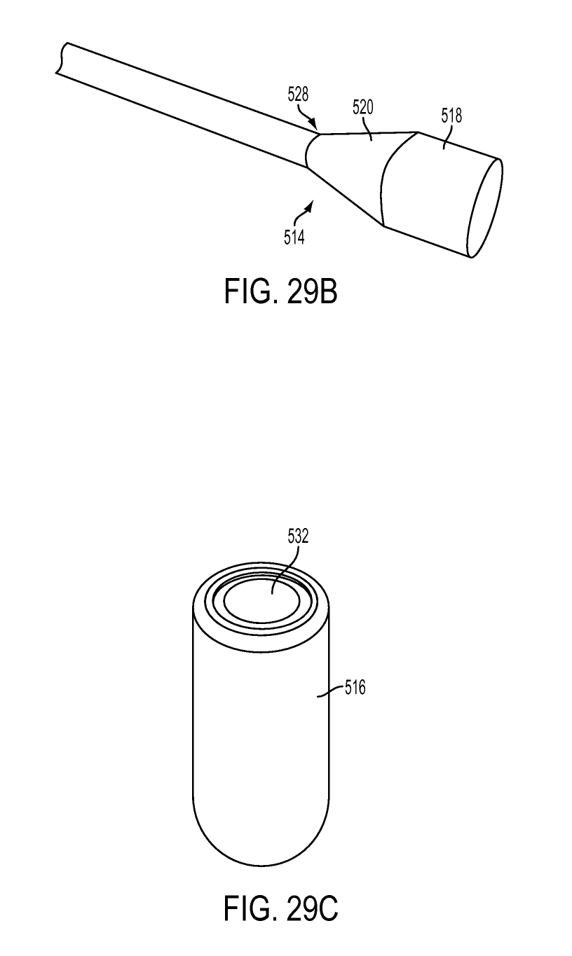

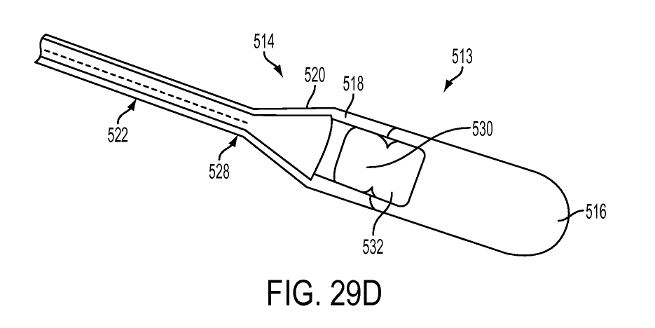

One embodiment of a pill catching system 513 utilizing the above magnetic base concept is illustrated in FIGS. 29A-29D. The pill catching system 513 can generally include an active magnetic catcher 514 and a magnetic pill 516. The catcher 514 can be configured to move between a first configuration in which it is magnetically attractive to the magnetic pill 516 and a second configuration in which it is not magnetically attractive to the pill 516.

As shown in FIG. 29B, the catcher 514 can have a substantially conical shaped housing 518 with a proximal portion 520 that gradually expands distally in diameter. The conical shape of the proximal portion 520 can be orientated in the direction of peristalsis to allow peristalsis to more easily move over the catcher 514 and thus to minimize its effect. The housing 518 can be coupled to a substantially flexible tether 522 extending from an anchoring ring 524 disposed within a patient's stomach 526. The tether 522 can extend past a patient's pylorus and into the duodenum, and the catcher 514 can be disposed on a distal end 528 of the tether 522.

In general, the catcher housing 520 can have a permanent magnet 530 disposed therein and the pill 516 can have a ferromagnetic core 532, as shown in FIGS. 29C and 29D. In some embodiments, the magnet 530 can be made of a material such as iron coated in chrome to prevent rusting in the G.I. tract. As the pill 516 passes through a patient's digestive tract, the catcher 514 can be in the first configuration such that the pill 516 is attracted to the magnet 530 within the catcher 514, as shown in FIG. 29D. In this configuration, peristalsis cannot act to remove the pill 516 from the catcher 514 due to the magnetic attraction. The magnet 530 can then be moved to the second configuration in which it is no longer attractive to the pill 516 such that the pill 516 is released from the catcher 514, allowing peristalsis to move the pill through the G.I. tract.

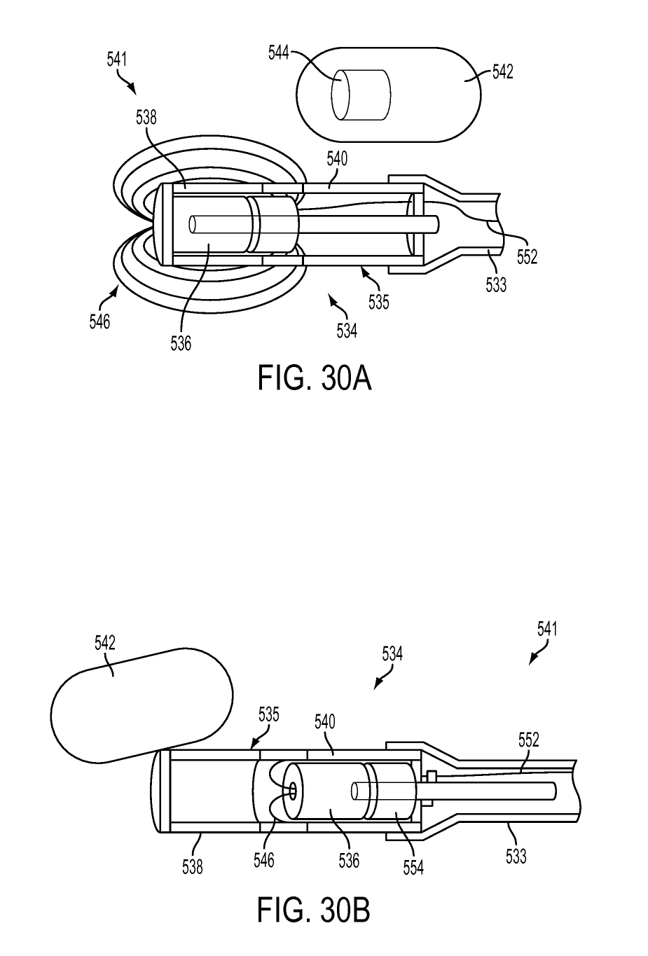

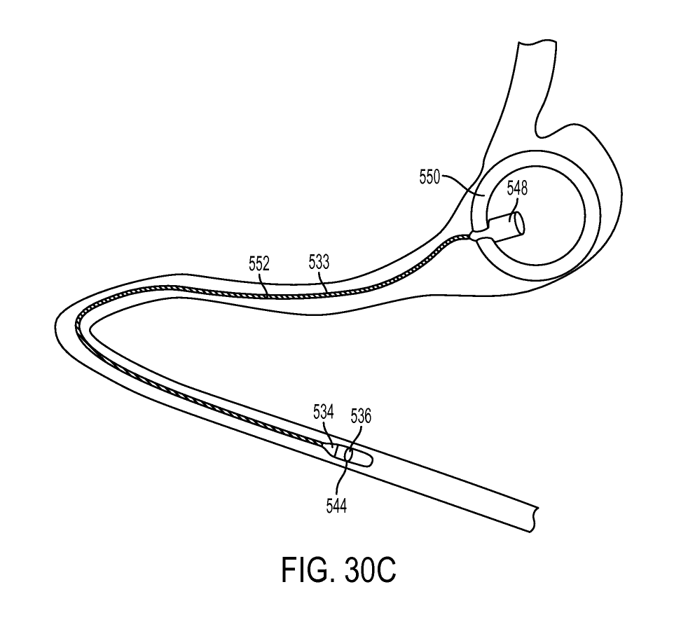

There are many ways to accomplish a catcher configurable between the first configuration and the second configuration. For example, as shown in FIGS. 30A and 30B, a pill catching system 541 is provided having a catcher 534 and a magnetic pill 542. The catcher 534 can have a housing 535 coupled to a tether 533. The housing 535 can be formed from a non-ferromagnetic distal portion 538 and a ferromagnetic proximal portion 540. These portions can each have any length as desired, and in the illustrated embodiment, they each form about half of a length of the housing 535. A magnet 536 can be disposed within the housing 535 and can be movable along a central longitudinal axis of the catcher 534 between the ferromagnetic portion 538 and the non-ferromagnetic portion 540. In the first configuration, the magnet 536 can be disposed adjacent to the non-ferromagnetic portion as shown in FIG. 30A such that its magnetic field 546 can extend outside of the catcher 534 to attract the pill 542 with a ferromagnetic core 544. When the magnet 536 is disposed adjacent to the ferromagnetic portion 540, the magnetic field 546 can be attenuated such that it does not extend outside and/or is trapped within the catcher 534 by the ferromagnetic portion 540 so that the catcher 534 is not magnetically attractive to the pill 542, as shown in FIG. 30A. In practice, the magnet 536 can be pulled proximally within the housing 535 such that its magnetic field 546 is attenuated to the point that it releases the pill 542.

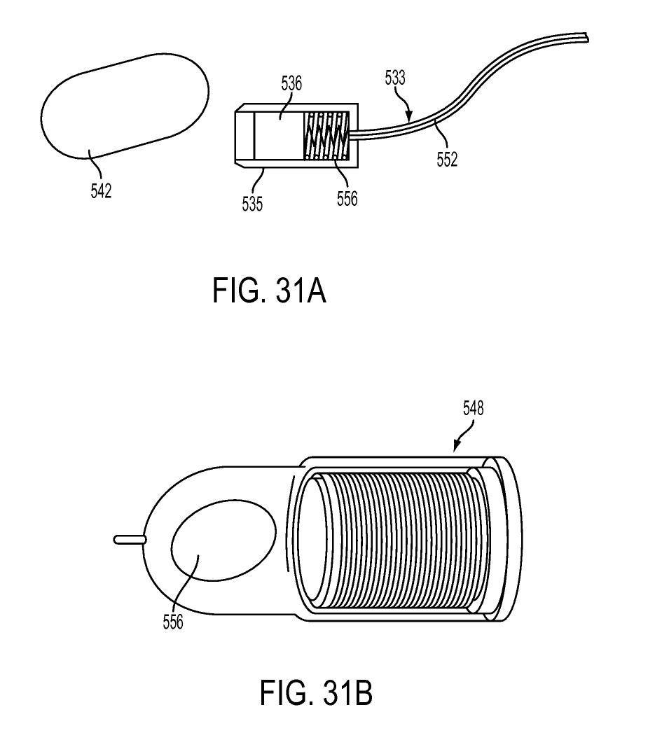

There are many ways in which to accomplish moving the magnet 536 axially within the housing 535 between the two configurations. As shown in FIGS. 30C and 31B, a TET coil 548 can be disposed on an anchor 550 within a patient's stomach. While there are many ways in which to couple the TET coil 548 to the anchor 550 and/or the tether 533, in the illustrated embodiment, the TET coil 548 can have an opening 556 disposed therein for receiving the anchor 550. The TET coil 548 can be slidable relative to the anchor 550 so that it can move with the tether 533 and its windings can intersect a magnetic field generated by an external hand held unit. The external magnetic field can induce a current in the TET coil 548 through inductive coupling. In this case, the TET coil 548 can have a wire 552 that extends through the tether 533 to transfer energy from the TET coil 548 to a motor 554, for example a stepper motor, disposed within the catcher housing 535. The motor 554 can be disposed adjacent to and/or in contact with the magnet 536 such that upon application of energy to the motor 554, it can rotate within the housing 535 to axially move the wire 552 within the tether 533, and thereby move the magnet 536 proximally and distally within the housing 535.

In another embodiment illustrated in FIG. 31A, a shape memory alloy (“SMA”) spring 556 such as a Nitinol spring, can be disposed within the catcher housing 535 adjacent to and/or in contact with the magnet 536. The TET coil 548 can transmit an electrical signal to the SMA 556, causing it to heat and change length, for example to contract in length, thereby moving the magnet 536 proximally within the housing 535. In other embodiments, a SMA wire can be coupled directly to the TET coil 548 near the anchor 550 and to a cord that extends through the tether 533 and couples to the magnet 536 (not shown). Upon application of electrical energy to the SMA wire, it can shorten, pulling on the cord and thereby moving the magnet 536 proximally within the housing 535. As will be appreciated, the SMA wire can also be disposed within the housing 535. In both cases, once electrical energy is removed from the SMA material, it will cool, moving the magnet 536 distally back to the first configuration so that it is ready to attract another pill to the catcher. It will be appreciated by those having ordinary skill in the art that there are many ways in which to axially move the magnet 536 within the housing 535, including but not limited to a reversible spring, mechanical pulley, servomechanism, optical means, Peltier device, inflatable bag, finger, etc.

In use, a patient can swallow the pill 542 having a dispensable therapeutic disposed inside. The catcher 534 can be in the first configuration and/or the “on” configuration such that the magnet 536 is disposed distally within the housing 535 adjacent to the non-ferromagnetic portion 538. The magnetic field 546 can thus extend outside of the housing 535 to attract ferromagnetic material. As the pill 542 passes through the patient's digestive tract and nears the catcher 534, it can be attracted to the magnetic field 546 of the magnet 536 within the catcher housing 535. The pill 542 can dock with the catcher 534 and can be configured to dispense its therapeutic. After a predetermined amount of time, or after an indication provided by the pill 542, the TET coil can provide electrical energy to the motor 554, the SMA 556, or other moving mechanism to cause the magnet 536 to be moved proximally within the housing 535 to the second configuration and/or the “off” configuration. As the magnet 536 is moved adjacent to the ferromagnetic portion 540 of the housing 535, it is moved farther from the pill 542 and its magnetic field 546 is attenuated. Once the magnetic field 546 is attenuated enough and/or is wholly contained within the housing 535, the catcher 534 no longer appears attractive to the pill 542 and peristalsis can remove the pill 542 from the catcher 534 and the pill 542 can pass out of the body. The magnet 536 can then be moved distally back to the first configuration such that it is ready to attract another pill to the catcher 534.

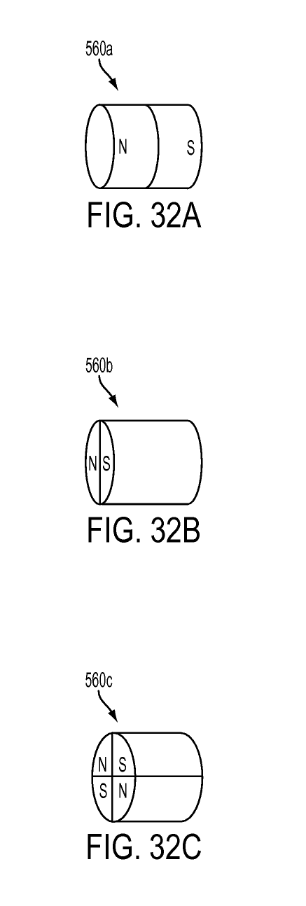

As will be appreciated by those having ordinary skill in the art, a number of magnet configurations can be utilized in the pill catching systems disclosed herein to produce a number of different magnetic field configurations. A few different configurations are illustrated in FIGS. 32A-32C. In FIG. 32A, a magnet 560a is provided having its north and south poles on opposite longitudinal ends thereof. Another magnet 560b is provided in FIG. 32B having its north and south poles on opposite lateral sides thereof. A magnet 560c is provided in FIG. 32C and can be divided laterally into four quarter sections with north and south poles alternating around the magnet 560c. There are many other magnet configurations possible and any of them can be utilized with any of the pill catching systems disclosed herein.

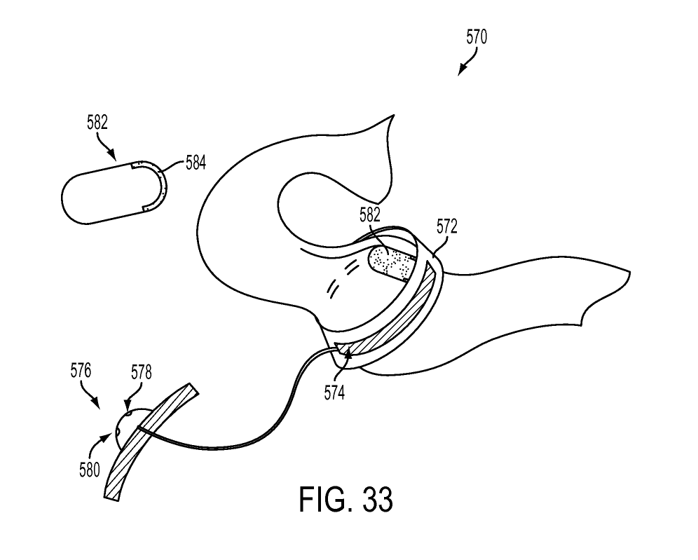

Another exemplary pill catcher system 570 is illustrated in FIG. 33. The pill catcher system 570 can have a catcher in the form of an electromagnetic band 572 that can be disposed around a lumen of a patient's G.I. tract. In some embodiments, the band 572 can be positioned around the duodenum portion of a patient's G.I. tract. The band 572 can have an electromagnetic strip 574 disposed thereon that can be electrically coupled to a subcutaneous port 576 positioned, for example, in a patient's abdomen. The port 576 can have an on switch 578 and an off switch 580 which the patient or other user can access for switching the strip 574 on and off. When the strip 574 is in the on configuration and/or the first configuration, it can generate a magnetic field within the patient's G.I. tract to attract a magnetic pill 582. The pill 582 can be configured to dispense a therapeutic at a specific location within the G.I. tract and can have a portion 584 formed from a ferromagnetic material so that it is attracted to the magnetic strip 574.

In use, a patient can swallow the magnetic pill 582. As the pill 582 passes near the strip 574, it is attracted to the strip 574 and can engage and be retained against the wall of the duodenum near the strip 574. Once the pill 582 completes dispensing its therapeutic, the patient or other user can use the port 576 to switch the strip 574 to the off configuration and/or the second configuration such that the magnetic field is removed. In other embodiments, the port can be configured to switch to the off configuration after a predetermined amount of time. The pill 582 can then release from the duodenum wall and can be passed through and out of the body.

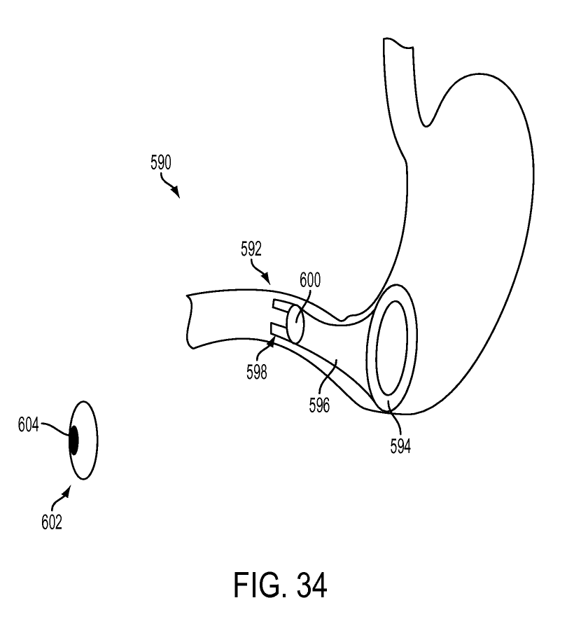

Another pill catcher system 590 is illustrated in FIG. 34. In this embodiment, a catcher 592 is provided that is coupled to an anchor 594. The anchor 594 can be a substantially flexible ring that can be endoscopically placed within a patient's stomach. The anchor 594 can have a size large enough so that it cannot be passed through a patient's pylorus and can have a sleeve 596 extending therefrom with a weighted distal portion 598. The sleeve 596 can have any length and width as desired, and the weighted distal portion 598 can be configured to allow peristalsis to move it distally within a patient's G.I. tract such that it hangs into the patient's duodenum or lower. In some embodiments, the sleeve 596 can be formed of a porous and/or fenestrated material with openings large enough to allow ingesta to pass through and/or to allow nutrients to be absorbed. The catcher 592 can include a magnet 600 positioned on a distal end of the sleeve 596 for attracting a pill. In this way, the anchor 594 can remain in the stomach while the catcher 592 hangs into duodenum via the sleeve 596 to catch a pill. In some embodiments, the magnet 600 can also form the weighted portion 598 of the sleeve 596. In other embodiments, the magnet 600 can be in addition to the weighted portion 598.

The pill catcher system 590 can also include a pill 602 having a ferromagnetic core 604 that can be attracted to the magnet 600 within the catcher 592. In some embodiments, the pill 602 can be coated in a hydrogel material, making the pill 602 slippery so that food and/or peristalsis does not pull the pill 602 off of the magnet 600. In use, after the pill 602 has docked with the magnet 600 in the distal end of the sleeve 596, it can dispense its therapeutic. After a predetermined amount of time, such as hours, days, or weeks, the hydrogel coating can dissolve, exposing a rough surface on the pill 602. The rough surface of the pill 602 allows food and/or peristalsis to grip the pill 602 and move it off the magnet 600 such that the pill 602 passes out of the body.

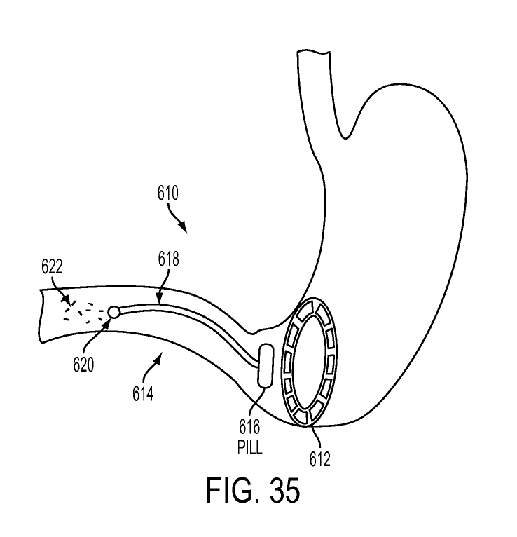

A further embodiment of a pill catcher system 610 is illustrated in FIG. 35. The pill catcher system 610 can include a catcher 612 in the form of a magnetic ring disposed within a patient's stomach and having a size such that it cannot pass through the patient's pylorus. The pill catcher system 610 can also include a pill package 614 formed of a pill 616 and a weighted catheter 618 attached to the pill 616. The pill 616 and the catheter 618 can initially have a dissolvable coating disposed therearound such that the catheter 618 and the pill 616 are in the form of a pill-shaped unit small enough to be swallowed. Once within a patient's stomach, the coating can dissolve, releasing the catheter 618. The catheter 618 can be coupled to the pill 616 such that it is configured to receive a therapeutic 622 dispensed from the pill 616 and to deliver the therapeutic through its distal end 620. The pill 616 can have a ferromagnetic portion that is attracted to the catcher 612.

In use, the pill package 614 can be swallowed by a patient. Once within the stomach of the patient, the coating around the pill package 614 can dissolve so that the pill 616 and the catheter 618 can separate. The pill 616 can be attracted to the catcher 612 and can engage the catcher 612 such that it does not pass through the pylorus. The weighted catheter 618 can pass through the pylorus and can hang into the patient's duodenum. As will be appreciated by those having ordinary skill in the art, the catheter 618 can have any length as desired such that the distal end 620 of the catheter can reach a specific location within a patient's G.I. tract. The pill 616 can be configured to dispense its therapeutic, which can pass through the catheter 618 and out its distal end 622 at the predetermined location within the G.I. tract. In some embodiments, the pill 616 and/or the catcher 612 can have a magnetic base switching mechanism as described above such that the pill 616 can be released from the catcher 612.

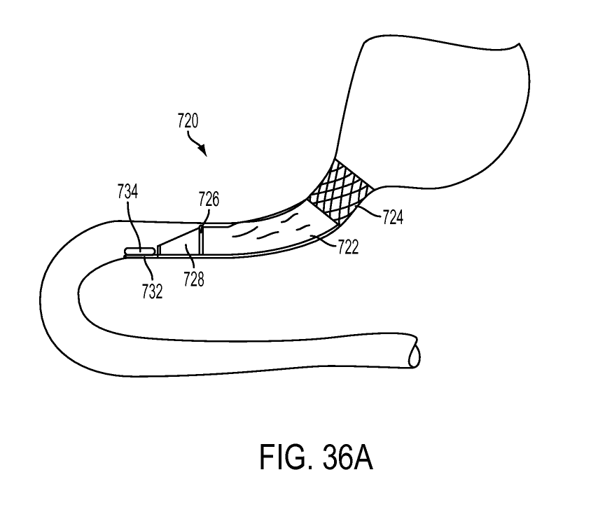

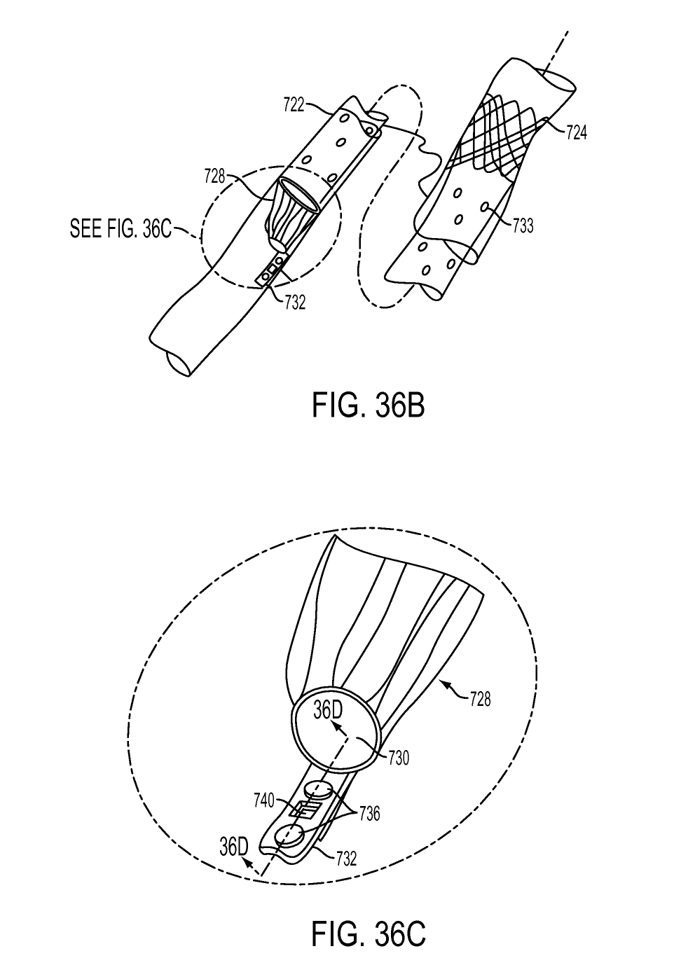

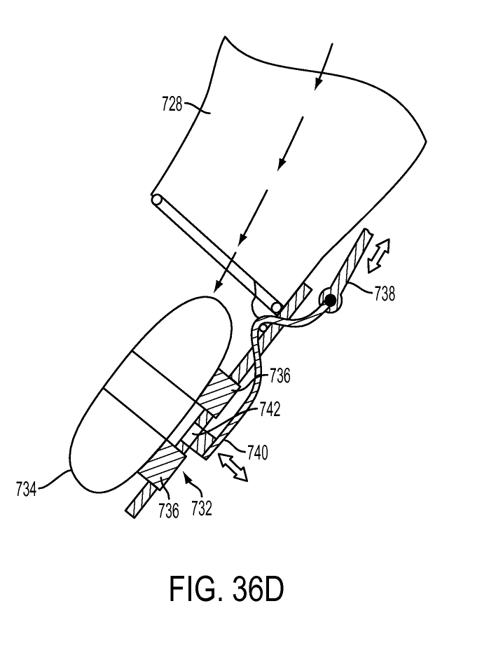

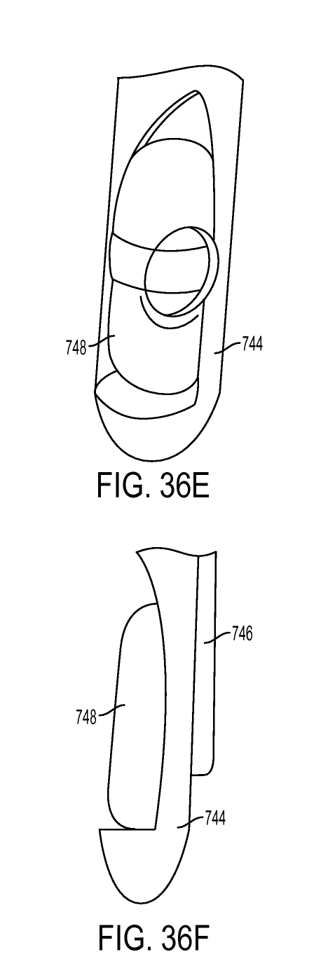

Another exemplary embodiment of a pill catching system 720 is illustrated in FIGS. 36A-36D. The pill catching system 720 can include a sleeve 722 anchored via a stent 724 or other anchoring mechanism within a patient's G.I. tract. A flexible funnel 728 can be disposed at a distal end 726 of the sleeve 722 and can have a flexible opening 730 that can allow chyme to pass unimpeded. In addition, the sleeve can optionally include a plurality of openings and/or perforations 733 to allow chime to pass and nutrients to be absorbed. The funnel 728 can open out into a catcher in the form of a catcher platform 732 and can be configured to funnel a pill 734 onto the platform 732, as shown in FIG. 36D. The pill 734 can have a ferromagnetic portion disposed therein and/or thereon, and the platform 732 can have two magnets 736 disposed thereon for attracting the ferromagnetic portion of the pill 734 in a first configuration. Once the pill 734 is docked on the platform 732, a Hall effect sensor or other mechanism can activate the pill 734 to dispense its therapeutic.

Once a desired amount of the therapeutic has been dispensed, a magnetic base type mechanism as described above can be used to redirect the field lines of the magnets 736 to move the catcher to a second configuration so that the pill 734 is released from the platform 732. For example, as shown in FIG. 36D, a pull cable 738 can be coupled to the platform 732 and/or the sleeve 722 and can have a spacer 740 mounted on a distal flexible lever 742 thereof. The spacer 740 can be formed from a non-ferrous material and can be configured to block the field lines of the magnets 736 when positioned between the magnets 736. An actuator (not shown), such as a solenoid powered by a battery, can be disposed within the patient's upper G.I. tract and/or the stomach and can pull on the cable 738 when actuated to cause the lever 742 to move the spacer 740 into an opening 742 in the platform 732 and between the magnets 736. In this way, the spacer 740 can block the field lines of the magnets 736, thereby causing the platform 732 to release the pill 734. Once the pill 734 is released, the spacer 740 can be removed from in between the magnets 736 so that the platform 732 is ready to receive another pill.

In an alternate embodiment shown in FIGS. 36E and 36F, a pull cable or another mechanism can by coupled directly to a magnet 746 positioned adjacent to a catcher platform 744 having a docked pill 748. The pull cable or other mechanism can retract the magnet 746 proximally away from the pill 748 such that magnetic force between the platform 732 and the pill 748 is weakened. Eventually, the pill 748 will no longer be attracted to the platform 732 and will be carried away from the catcher platform 744 by peristalsis. The magnet 746 can then be moved distally to its original position so that the platform 744 is ready to receive another pill.

Passive Magnetic Catcher

In some embodiments, exemplary pill catcher systems can include a passive catcher. Passive catchers can be advantageous in that they do not have moving parts and/or do not require electrical power and/or external signals to operate. Such a system can be easier to implant and/or to maintain once implanted. The following embodiments generally involve passive catchers that rely on magnetism to retain a pill.

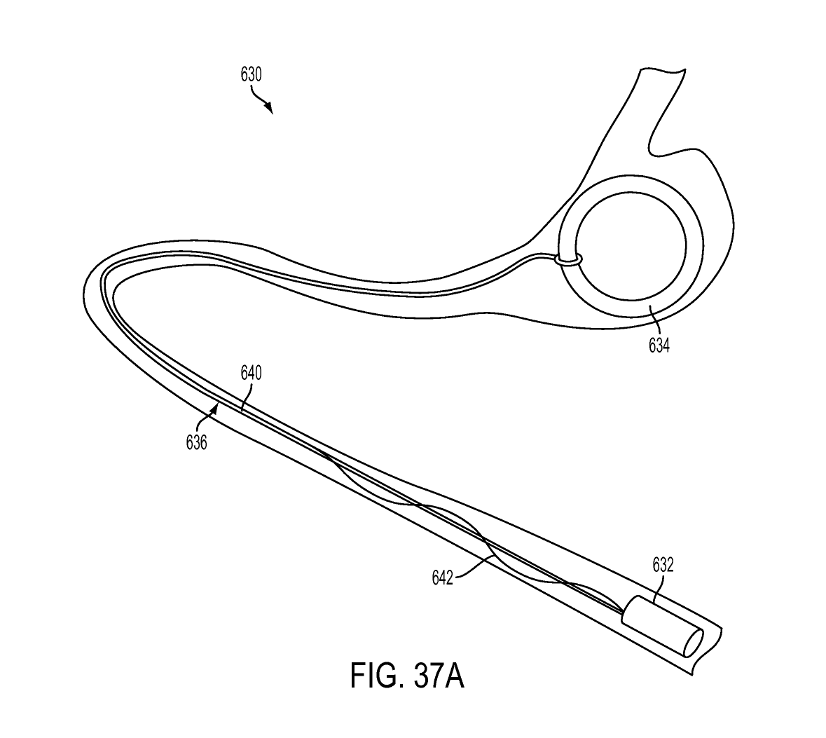

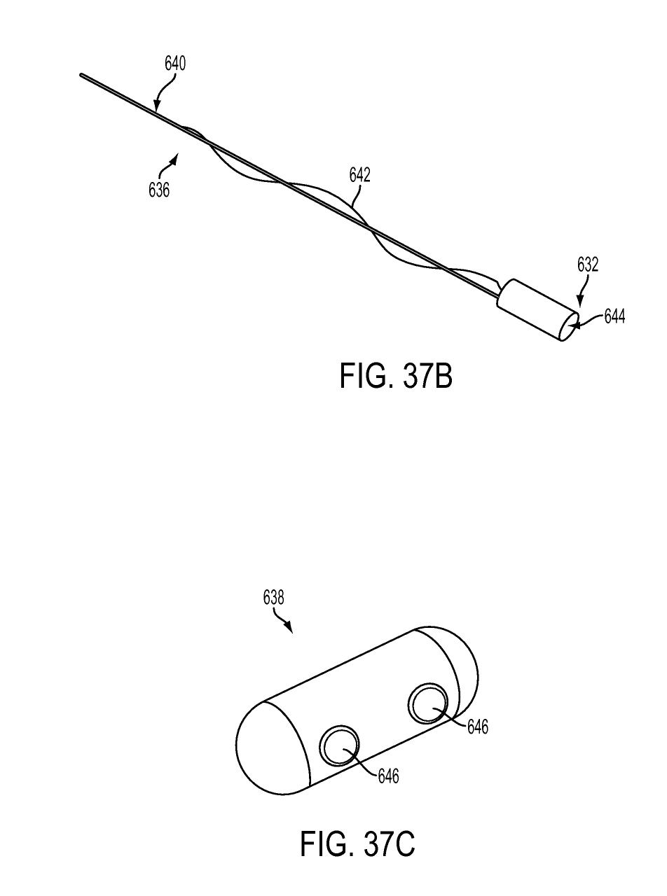

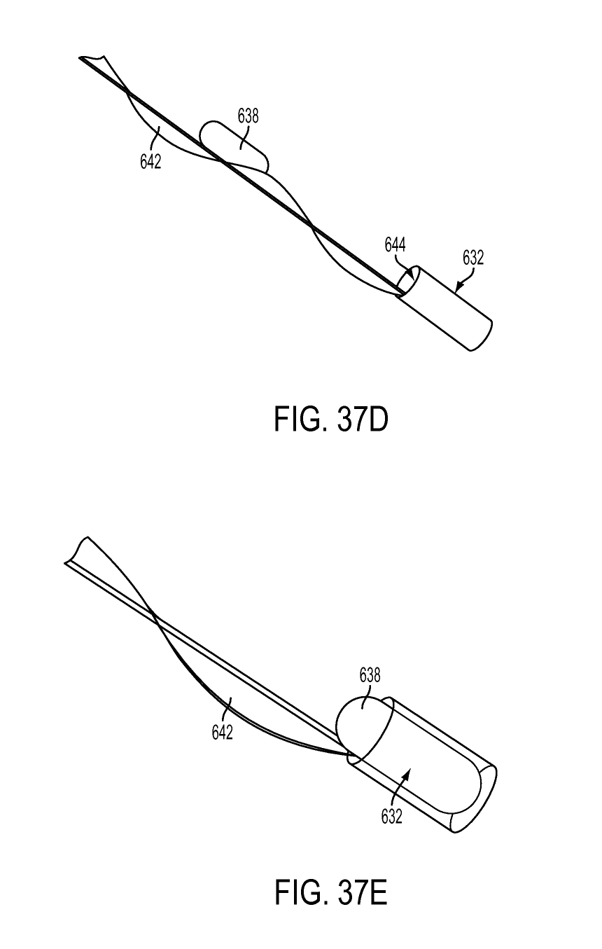

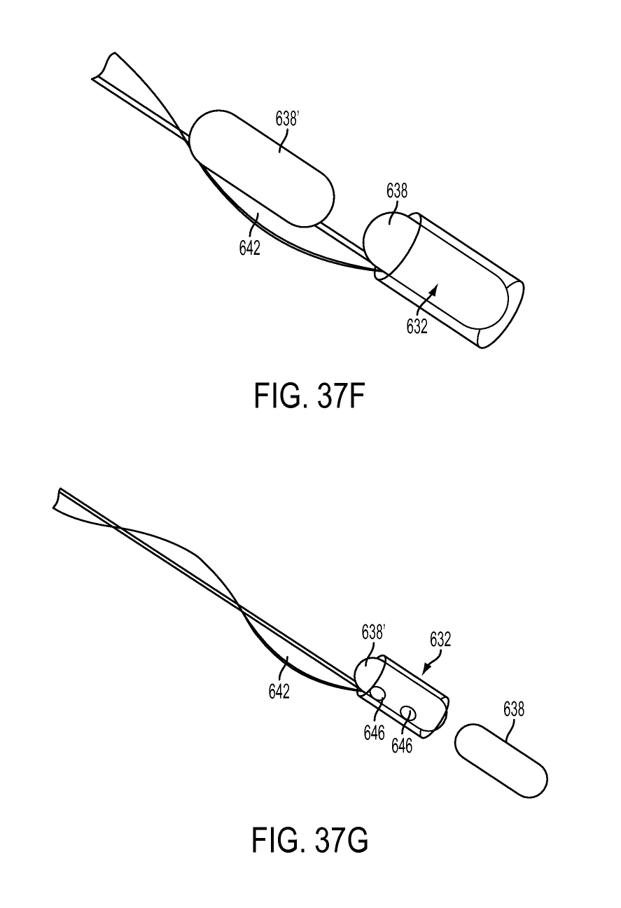

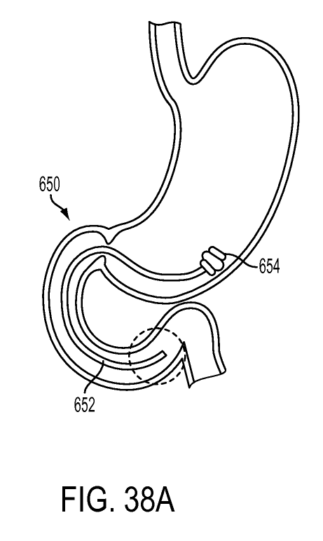

In one embodiment illustrated in FIGS. 37A-37G, a pill catcher system 630 is provided and can include a catcher 632 and a pill 638. The catcher 630 can be coupled to an anchor 634 by a tether 636. While the anchor 634 can take many forms, in the illustrated embodiment the anchor 634 is in the form of a stomach ring as previously disclosed herein. The tether 636 can be formed of a substantially straight proximal portion 640 and a helical distal portion 642 that terminates distally at the catcher 632. The catcher 632 can be in the form of a substantially cylindrical sleeve having an opening 644 with a diameter of a size sufficient to receive the pill 638. In some embodiments, the catcher 632 can have one or more openings formed therein (not shown) to prevent entrapment of food particles when no pill is disposed within the catcher 632. The helical portion 642 of the tether 636 can be formed of a magnetic material and/or can be configured to axially align and funnel the pill 638 into the opening 644 of the catcher 632. More particularly, the pill 638 can have one or more magnetic portions 646 disposed therein that can be attracted to the magnetic helical portion 642 of the tether 636 so that the pill 638 can travel down the helical portion 642.

In use, as shown in FIGS. 37D and 37E, after the pill 638 is swallowed and passes through a patient's pylorus, it can be attracted to and/or guided by the magnetic helical portion 642 of the tether 636. The pill 638 can travel distally along the helical portion 642 under peristalsis until it reaches, and is funneled into, the opening 644 in the catcher 632. Once within the substantially rigid catcher 630, peristalsis can no longer move the pill 638 and it can remain within the catcher 632 to dispense its therapeutic. The pill 638 can be retained within the catcher through the use of magnetic attraction, spring force, friction, or other mechanism. The pill 638 can remain within the catcher 632 until a second pill 638′ pushes the pill 638 out of the catcher 630 as shown in FIGS. 37F and 37G. A Hall effect sensor or other mechanism can serve to notify the pill 638 that it no longer needs to dispense its therapeutic once it has been ejected from the catcher 638.

In some embodiments, the helical portion 642 of the tether 636 can have a lead-in ramp or other alignment mechanism adjacent to the catcher 630 to help align the pill 638 before it enters the catcher 630. In addition, the pill can optionally have an alignment feature, such as a rib, formed thereon to align the pill end-to-end along the tether 636. Further, the pill 638 can optionally have flattened end surfaces. The flattened surfaces can aid in keeping the second pill 638′ aligned as it pushes the pill 638 out of the catcher 638. As will be appreciated, there are many other alignment features and mechanisms that can be used with the pill catcher system 630.

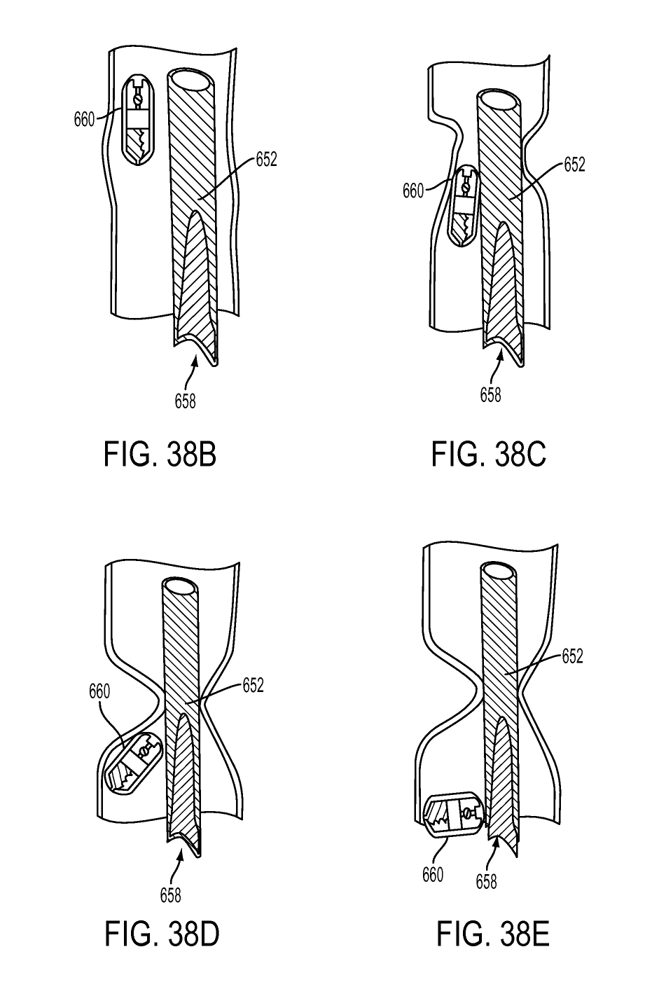

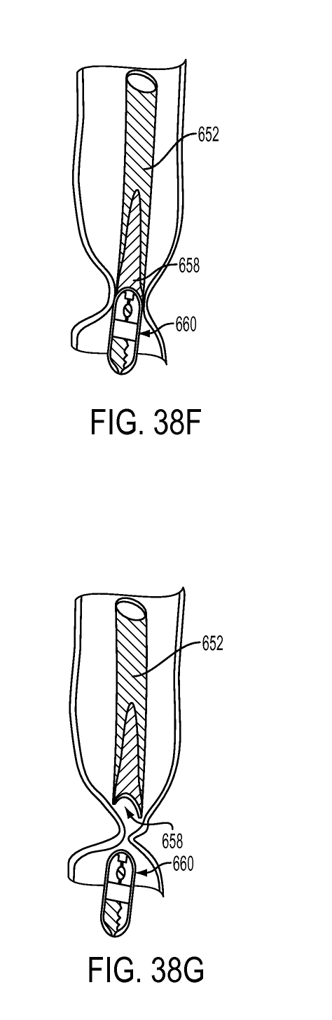

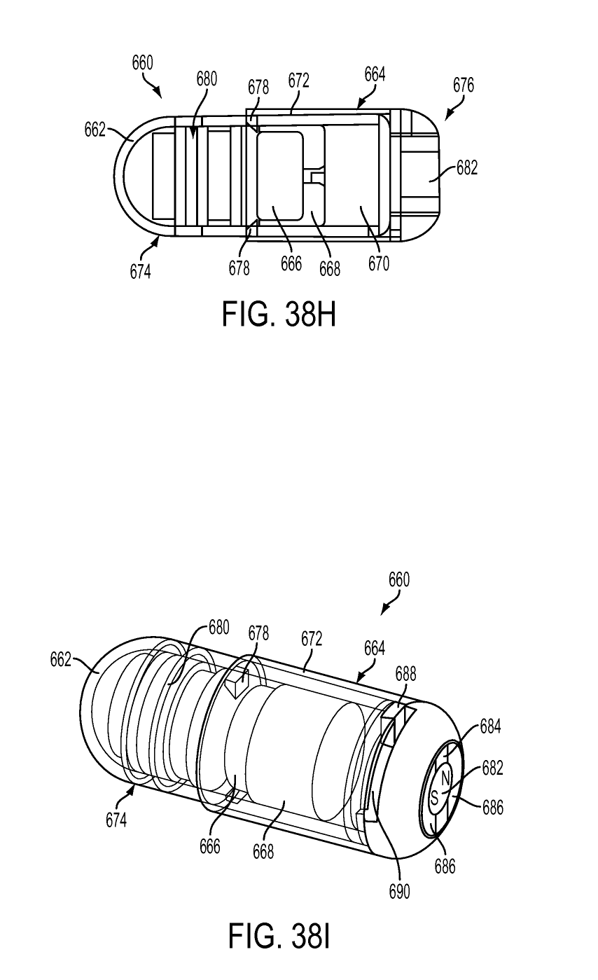

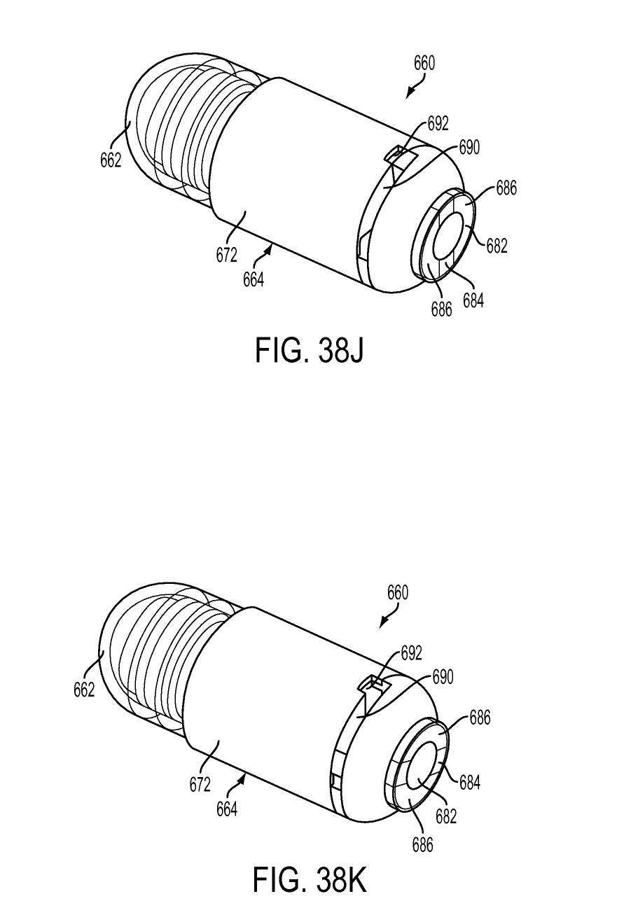

Another pill catcher system 650 is provided in FIGS. 38A-38G. The pill catcher system 650 can include a catcher 652 extending from an anchor 654 disposed within a patient's stomach. The anchor 654 can take many forms, but in the illustrated embodiment, the anchor 654 can be a staple or other fixation mechanism 656 that fixes a proximal end of the tether to the stomach wall. The catcher 652 can be in the form of an elongate, substantially flexible weighted sleeve that can extend into a patient's G.I. tract past the pylorus. The catcher 652 can have a substantially concave distal end 658 that is formed of a ferromagnetic material that can appear attractive to a magnetic pill. The pill catcher system 650 can also include a pill 660 having a magnet disposed therein. In use, as the pill 660 travels through a patient's G.I. tract, it moves with peristalsis along the catcher 652 as shown in FIGS. 38B-38E. As the pill nears the catcher's distal end 658, it can be attracted to the magnetic portion thereof and it can dock and magnetically engage with the distal end 658 as shown in FIG. 38F. The pill 660 can then dispense its therapeutic at the predetermined location within the G.I. tract.

While there are many different types of magnetic pills that can dock with the catcher 658, a magnetic base mechanism associated with the pill 660 is shown in more detail in FIGS. 38H-38K. The pill 660 can include a pill capsule body 662 and a magnetic sheath 664. The body 662 can contain a therapeutic reservoir 670 and mechanisms for dispensing the therapeutic, such as a battery 680, a battery operated stepper motor 666, and a piston 668. The magnetic sheath 664 can have a sheath portion 672 that extends around an outside surface 674 of the pill body 662 and a magnet portion 676 coupled to one end of the pill body 662. The sheath portion 672 can have fingers 678 that extend into the pill body 662 and engage the piston 668 such that as the piston 668 moves in a second direction, described in detail below, the sheath 664 is caused to move with the piston 668.

In some embodiments, the magnet portion 676 can include a magnetic core 682, a non-ferrous spacer 684, and two ferromagnetic portions 686. The magnet portion 676 can also include a bar 688 coupled to the spacer 684 and the ferromagnetic portions 686. The magnetic core 682 can have its poles oriented such that it is in the “on” and/or first configuration in the illustrations shown in FIGS. 38I and 38J. In this orientation, a magnetic field of the magnetic core 682 extends out of the pill 660 and can allow the pill 660 to be attracted to and dock with a ferromagnetic material within a catcher, for example the ferromagnetic material within the catcher 652 shown in FIG. 38A. The bar 688 can be configured to rotate with the spacer 684 and the ferromagnetic portions 686 relative to the sheath 664 and the pill body 662 via a slot 690 formed in the sheath 664. In the first configuration, the magnet portion 676 wants to move and/or is biased to the off configuration and/or second configuration since that is its lowest energy state. The bar 688 can be restrained from rotating to the second configuration by an opening 692 formed at one end of the slot 690 in the sheath 662. In use, the sheath 664 remains stationary and the pill 660 remains in the first configuration as the stepper motor 666 moves the piston 668 in a first direction to dispense a therapeutic held in the reservoir 670. Once a desired amount of therapeutic is dispensed and/or at any time before or after a therapeutic has been dispensed, the stepper motor 666 can move the piston 668 in a second direction, opposite to the first direction, and can engage the fingers 678 to thereby move the sheath 664 axially along the pill body 662 in the second direction. The bar 688 remains axially stationary such that it moves into the slot 690 as shown in FIG. 38J as the sheath 664 moves in the second direction. Due to the magnetic biasing, the bar 668 will rotate relative to the sheath 664 and the pill body 662 to the second configuration as shown in FIG. 38K, thereby turning “off” the magnetic field, i.e., the magnetic field is now contained within the ferromagnetic portions 686. Thus, in the case of FIGS. 38A-38E, the pill 660 is no longer retained against the distal end 658 of the catcher 652 and peristalsis can move the pill 660 further along the G.I. tract such that the pill 660 passes out of the body.

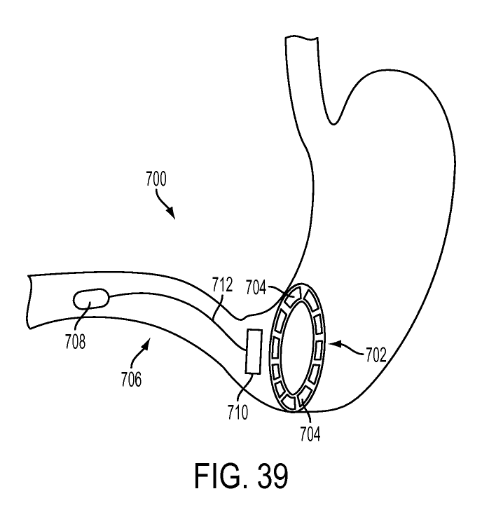

Another exemplary embodiment of a pill catcher system 700 is illustrated in FIG. 39. The pill catcher system 700 can have catcher in the form of a ring 702 disposed within a patient's stomach. The ring 702 can have a size such that it cannot be passed through a patient's pylorus. The ring 702 can be substantially rigid or substantially flexible and can be formed of metallic and/or polymeric material. In some embodiments, the ring 702 can have one or more ferromagnetic portions 704 disposed thereon and/or therein. The pill catcher system 700 can also include a pill package 706 having a pill 708 configured to dispense a therapeutic and an anchor hook 710 coupled to the pill by a tether 712. The pill 708, the anchor hook 710, and the tether 712 can initially be bound together as a unit within a dissolvable coating so that the pill package 706 can be swallowed. As the pill package 706 travels through the body, the coating can dissolve so that the pill 708, tether 712, and anchor hook 710 can separate from one another.

The anchor hook 710 can have many configurations, but in the illustrated embodiment it can have a magnet disposed therein for engaging the ring 702. As the anchor hook 710 engages the ring 702, the tether 712 can allow the pill 708 to pass into the duodenum or lower so that the pill 708 can deliver its therapeutic at a predetermined location, i.e., at a location determined by a length of the tether 712. The anchor hook 710 can also include a magnetic base switching mechanism as described above that can switch from the first configuration and/or the on configuration to the second configuration and/or the off configuration in response to an external signal and/or after a predetermined amount of time. As with the other magnetic base embodiments described herein, when the anchor hook 710 is in the first configuration, it is magnetically attractive and can thus engage with the ferromagnetic portions 704 in the ring 702. In the second configuration, the magnetic field of the magnet within the anchor hook 710 is contained such that the anchor hook 710 is not attracted to the ring 702 and thus releases from the ring 702, allowing the pill, the tether, and the hook to pass out of the body.

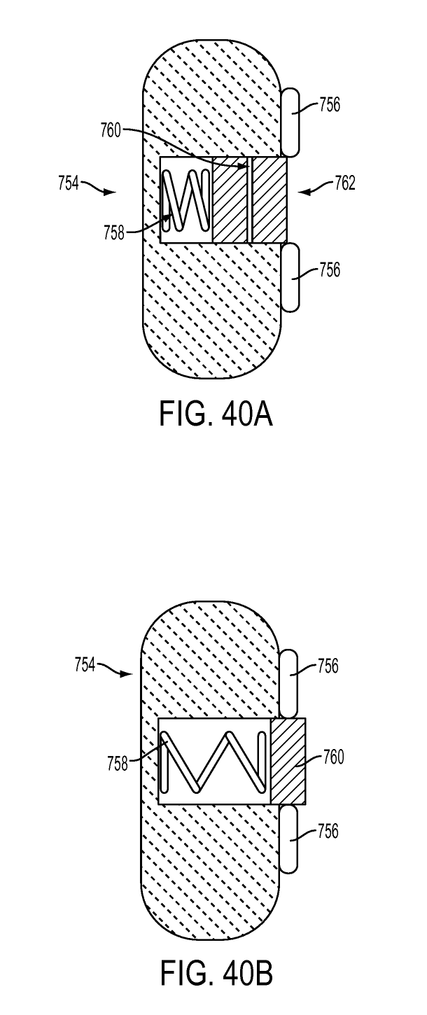

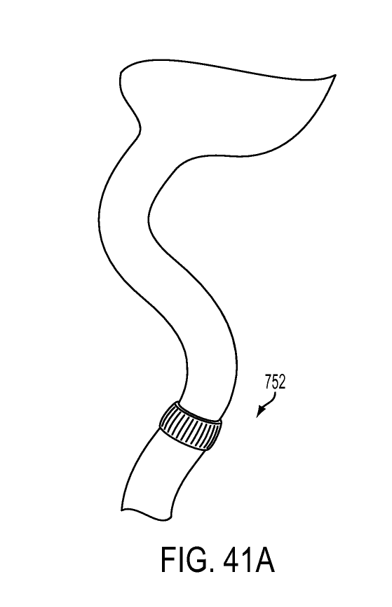

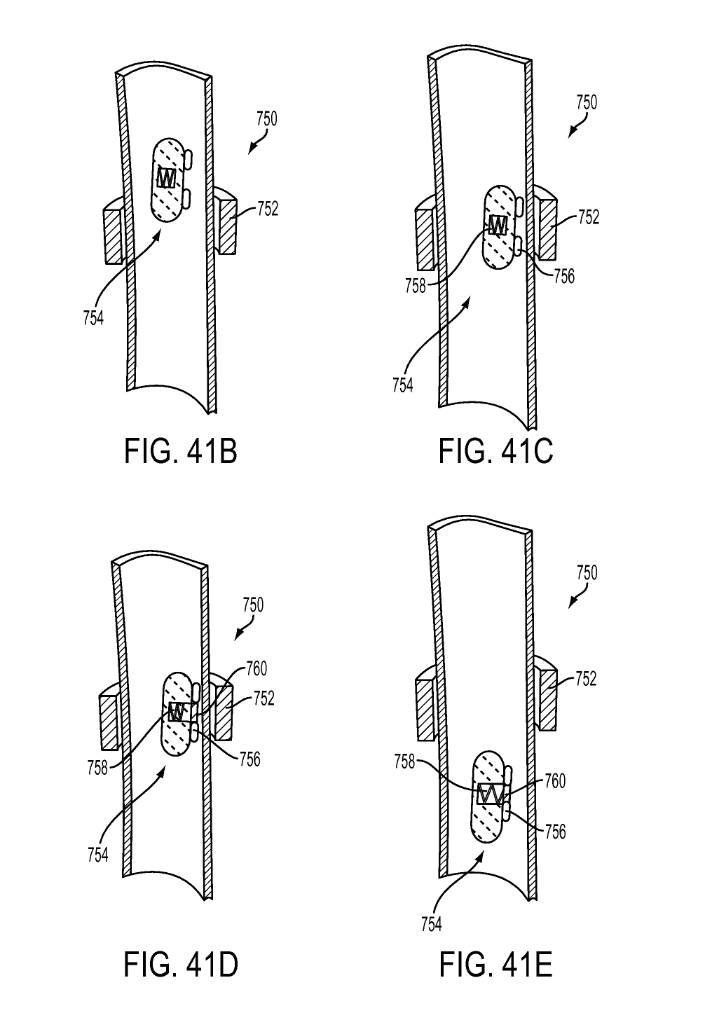

Another exemplary embodiment of a pill catcher system 750 is shown in FIGS. 40A-40B, and 41A-41E. The pill catcher system 750 can include catcher in the form of a ferromagnetic band 752 disposed around a lumen of a patient's G.I. tract as shown in FIG. 41A. In addition, the pill catcher system 750 can include a pill 754 having two magnets 756 disposed thereon. FIG. 40A shows the pill in the first configuration and/or the on configuration in which the magnets 756 can be attracted to the ferromagnetic band 752 as the pill travels through the patient's G.I. tract. Once in the vicinity of the band 752, the pill 754 can dock against the lumen wall near the band 752 and can be configured to dispense its therapeutic.

As shown most clearly in FIGS. 40A and 40B, the pill 754 can also include a spring 758 and a spacer 760 disposed within an interior thereof. The spring 758 can be held in a compressed condition in the first configuration by a dissolvable and/or absorbable coating 762. The coating 762 can remain in place for a time sufficient for the pill 754 to dock with the band 752 and dispense its therapeutic as shown in FIG. 41C. Once the coating 762 dissolves, the spring 758 can move to an uncompressed condition and can move the spacer 760 between the magnets 756, reducing and/or eliminating the magnetic field between the magnets 756 as shown in FIG. 41D. The pill 754 is now in the second configuration and/or the off configuration. Peristalsis can now move the pill 754 away from the band 752, as shown in FIG. 41E, because of the reduced and/or eliminated magnetic attraction between the pill 754 and the band 752.

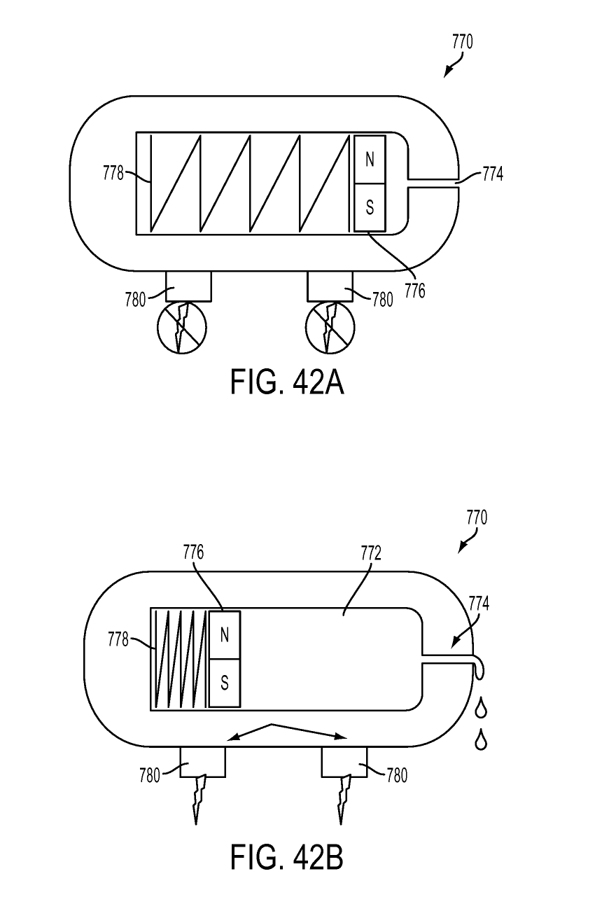

Another embodiment of a pill 770 that can be used with the ferromagnetic band 752 is illustrated in FIGS. 42A-42B. The pill 770 can have a reservoir 772 disposed therein for containing a therapeutic to be dispensed once the pill 770 is docked with the band 772. The pill 770 can also include an opening 774 in one end for dispensing the therapeutic. The pill 770 can further include a magnet 776 disposed in its interior coupled to a spring 778. The magnet 776 and the spring 778 can act as a plunger to dispense the therapeutic once the pill 770 is docked with the band 772. As will be appreciated in the art, there are many other mechanisms to move the magnet 776 within the pill 770 including power screws, motors, and others which have been described herein.

To facilitate docking with the band 752, the pill 770 can include two ferromagnetic feet 780 that can act with the magnet 776 to attract the pill 770 to the band 752 in the first configuration and/or on configuration as shown in FIG. 42B. As the magnet 776 moves along the central longitudinal axis of the pill 770 to dispense the therapeutic, the magnetic circuit becomes complete as shown in FIG. 42A, and the pill 770 is moved to the off configuration and/or the second configuration. The feet 780 are no longer magnetically attracted to the band 752 and the pill 770 releases from the band 752 so that peristalsis can move it through the G.I. tract.

As will be appreciated by those skilled in the art, any and all of the embodiments disclosed herein can be interchangeable with one another as needed. For example, a pill catcher kit could be provided and could include multiple anchors, tethers, pills, etc. having different sizes, configurations, medications, dosages, etc. as needed in particular application.

In addition, the devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device for controlled therapeutic drug delivery within a patient, comprising:
an anchor configured to be disposed within a patient's digestive tract and having a catch mechanism coupled thereto, the catch mechanism having a first configuration in which a magnet on the catch mechanism is configured to magnetically engage a pill swallowed by a patient to prevent passage of the pill through the catch mechanism, and a second configuration in which the magnet is configured to release the pill to allow passage of the pill through the catch mechanism.

2. The device of claim 1 further comprising a tether extending between the anchor and the catch mechanism.

3. The device of claim 1, wherein the catch mechanism comprises a housing having proximal and distal end with a longitudinal axis extending therebetween, and wherein the magnet is disposed within the housing and is movable along the longitudinal axis of the housing.

4. The device of claim 3, wherein, in the first configuration the magnet is disposed distally within the housing to attract a pill and prevent passage of the pill, and in the second configuration the magnet is disposed proximally within the housing to release the pill and allow it to pass.

5. The device of claim 1, wherein the catch mechanism includes a shape memory alloy disposed therein and configured to move the magnet between the first configuration and the second configuration in response to a change in energy application thereto.

6. The device of claim 1, wherein the catch mechanism includes a step motor disposed therein and configured to move the magnet between the first configuration and the second configuration in response to a change in energy application thereto.

7. The device of claim 1, wherein the catch mechanism includes a transcutaneous energy transfer coil configured to receive an external signal to supply energy to a mechanism for moving the magnet between the first configuration and the second configuration.

8. The device of claim 3, wherein the housing includes a distal non-ferromagnetic portion configured to allow a magnetic field of the magnet to extend outside of the housing when the catch mechanism is in the first configuration to attract a pill, and a proximal ferromagnetic portion configured to prevent the magnetic field from extending outside of the housing when the catch mechanism is in the second configuration to prevent attraction of a pill.

9. A system for controlled therapeutic drug delivery within a patient, comprising:
a pill configured to deliver at least one therapeutic drug and configured to be swallowed by a patient; and
a catch mechanism configured to be disposed within a patient's digestive tract, the catch mechanism being movable between a first configuration in which the catch mechanism is configured to magnetically engage the pill and prevent passage of the pill through the catch mechanism, and a second configuration in which the catch mechanism is configured to release the pill to allow passage of the pill through the catch mechanism.

10. The system of claim 9 wherein the pill includes a ferromagnetic material configured to be magnetically engaged with the catch mechanism in the first configuration.

11. The system of claim 9, further comprising an anchor configured to be disposed within a patient's digestive tract and configured to retain the catch mechanism within the patient's digestive tract.

12. The system of claim 11, wherein the anchor comprises a substantially rigid ring configured to be disposed within a patient's stomach and having a size large enough to prevent passage of the ring through a patient's pylorus.

13. The system of claim 9, wherein the catch mechanism comprises a magnet disposed within a housing, and wherein, in the first configuration the magnet is disposed distally within the housing to magnetically engage the pill and prevent passage of the pill, and in the second configuration the magnet is disposed proximally within the housing to release the pill and allow it to pass.

14. The system of claim 9 wherein the catch mechanism includes a distal non-ferromagnetic portion configured to allow a magnetic field of a magnet to extend outside of the catch mechanism when in the first configuration to attract the pill, and a proximal ferromagnetic portion configured to prevent the magnetic field from extending outside of the catch mechanism when in the second configuration to prevent attraction of the pill.

15. A method for controlled therapeutic drug delivery, comprising:
implanting an anchor having a catch mechanism in a patient's digestive tract, the catch mechanism being positioned in a first configuration in which the catch mechanism is effective to magnetically engage a pill and prevent passage of the pill through the catch mechanism, and the catch mechanism being movable to a second configuration in which the catch mechanism releases the pill to allow passage of the pill through the catch mechanism.

16. The method of claim 15, wherein the catch mechanism comprises a magnet that moves within a housing to move between a first configuration and a second configuration.

17. The method of claim 16, wherein a magnetic field of the magnet extends outside of the catch mechanism in the first configuration and is prevented from extending outside of the catch mechanism in the second configuration.

18. The method of claim 17, further comprising actuating an external source to supply energy to the catch mechanism to move the catch mechanism between the first configuration and the second configuration.

19. The method of claim 15, further comprising introducing a pill orally into a digestive tract, the pill engaging the catch mechanism.

20. The method of claim 19, wherein the pill delivers a therapeutic drug directly into the patient's digestive tract when engaged with the catch mechanism.
